SEQUENCE LISTING (14 sequences)

SEQ ID NO: 1 - protein, 5 aa, Mus musculus
NYWMH

SEQ ID NO: 2 - protein, 17 aa, Mus musculus
ATYRGHSDTYYNQKFKG

SEQ ID NO: 3 - protein, 12 aa, Mus musculus
GAIYDGYDVLDN

SEQ ID NO: 4 - protein, 11 aa, Mus musculus
SASQDISNYLN

SEQ ID NO: 5 - protein, 7 aa, Mus musculus
YTSNLHS

SEQ ID NO: 6 - protein, 9 aa, Mus musculus
QQYRKLPWT

SEQ ID NO: 7 - protein, 121 aa, Artificial Sequence
  Artificially Synthesized Sequence
QVQLVQSGAEVKKPGSSVKVSCKASGGTFSNYWMHWVRQAPGQGLEWMGATYRGHSDTYYNQKFKGRVTITADKSTSTAYMELSSLRSEDTAVYYCARGAIYDGYDVLDNWGQGTLVTVSS

SEQ ID NO: 8 - protein, 108 aa, Artificial Sequence
  Artificially Synthesized Sequence
DIQMTQSPSSLSASVGDRVTITCSASQDISNYLNWYQQKPGKAPKLLIYYTSNLHSGVPSRFSGSGSGTDFTLTISSLQPEDFATYYCQQYRKLPWTFGQGTKLEIKR

SEQ ID NO: 9 - protein, 451 aa, Artificial Sequence
  Artificially Synthesized Sequence
QVQLVQSGAEVKKPGSSVKVSCKASGGTFSNYWMHWVRQAPGQGLEWMGATYRGHSDTYYNQKFKGRVTITADKSTSTAYMELSSLRSEDTAVYYCARGAIYDGYDVLDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 214 aa, Artificial Sequence
  Artificially Synthesized Sequence
DIQMTQSPSSLSASVGDRVTITCSASQDISNYLNWYQQKPGKAPKLLIYYTSNLHSGVPSRFSGSGSGTDFTLTISSLQPEDFATYYCQQYRKLPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 451 aa, Artificial Sequence
  Artificially Synthesized Sequence
QVQLVQSGAEVKKPGSSVKVSCKASGGTFSNYWMHWVRQAPGQGLEWMGATYRGHSDTYYNQKFKGRVTITADKSTSTAYMELSSLRSEDTAVYYCARGAIYNGYDVLDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 451 aa, Artificial Sequence
  Artificially Synthesized Sequence
QVQLVQSGAEVKKPGSSVKVSCKASGGTFSNYWMHWVRQAPGQGLEWMGATYRGHSDTYYNQKFKGRVTITADKSTSTAYMELSSLRSEDTAVYYCARGAIYDGYDVLDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESDGQPENNYKTTPPVLDSDGSFFLYSKLTVDKSRWQQGNVFSCSVMHEALHNHYTQKSLSLSPGK

SEQ ID NO: 13 - protein, 451 aa, Artificial Sequence
  Artificially Synthesized Sequence
QVQLVQSGAEVKKPGSSVKVSCKASGGTFSNYWMHWVRQAPGQGLEWMGATYRGHSDTYYNQKFKGRVTITADKSTSTAYMELSSLRSEDTAVYYCARGAIYDGYDVLDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPEDNYKTTPPVLDSDGSFFLYSKLTVDKSRWQQGNVFSCSVMHEALHNHYTQKSLSLSPGK

SEQ ID NO: 14 - protein, 450 aa, Artificial Sequence
  Artificially Synthesized Sequence
VQLVQSGAEVKKPGSSVKVSCKASGGTFSNYWMHWVRQAPGQGLEWMGATYRGHSDTYYNQKFKGRVTITADKSTSTAYMELSSLRSEDTAVYYCARGAIYDGYDVLDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESDGQPEDNYKTTPPVLDSDGSFFLYSKLTVDKSRWQQGNVFSCSVMHEALHNHYTQKSLSLSPGK

(19) United States
(12) Patent Application Publication
(10) Pub. No.: US 2022/0289859 A1
(43) Pub. Date: Sep. 15, 2022

(54) Biopharmacuetical Compositions and Related Methods

(71) Applicant: GlaxoSmithKline Intellectual Property Development Limited, Brentford (GB)

(72) Inventors: James K. Kranz, Harvard, MA (US); Michael Joseph Molloy, Stevenage (GB); Joseph V. Rinella, Jr., Collegeville, PA (US); Elizabeth Rae Schmidt, King Of Prussia, PA (US); Hillary Amber Schuessler, King Of Prussia, PA (US); Tejash Shah, Ware (GB)

(21) Appl. No.: 17/633,115

(22) PCT Filed: Jul. 31, 2020
(86) PCT No.: PCT/IB20/57267

§ 371 Date: Feb. 4, 2022

Related U.S. Application Data
(60) Provisional application No. 62/984,110, filed on Mar. 2, 2020.
(60) Provisional application No. 62/948,432, filed on Dec. 16, 2019.
(60) Provisional application No. 62/883,451, filed on Aug. 6, 2019.

(51) Int. Cl. C07K 16/28; A61P 35/00

(52) U.S. Cl. CPC C07K 16/2878; A61P 35/00; A61K 2039/505

(57) ABSTRACT

The invention described herein provides compositions comprising anti-BCMA antigen binding proteins and related methods for treating BCMA mediated diseases or disorders.

### SEQUENCE LISTING

The instant application contains a Sequence Listing which has been submitted electronically in ASCII format and is hereby incorporated by reference in its entirety.

### FIELD OF THE INVENTION

The invention described herein provides compositions comprising anti-BCMA antigen binding proteins and related methods for treating BCMA-mediated diseases or disorders.

### BACKGROUND OF THE INVENTION

BCMA (CD269 or TNFRSF17) is a member of the TNF receptor superfamily. It is a non-glycosylated integral membrane receptor for the ligands BAFF and APRIL. BCMA's ligands can also bind additional receptors: TACI (Transmembrane Activator and Calcium modulator and cyclophilin ligand Interactor), which binds APRIL and BAFF; as well as BAFF-R (BAFF Receptor or BR3), which shows restricted but high affinity for BAFF. Together, these receptors and their corresponding ligands regulate different aspects of humoral immunity, B-cell development and homeostasis.

BCMA's expression is typically restricted to the B-cell lineage and is reported to increase in terminal B-cell differentiation. BCMA is expressed by human plasma blasts, plasma cells from tonsils, spleen and bone marrow, but also by tonsillar memory B cells and by germinal center B cells, which have a TACI-BAFFR low phenotype (Darce et al., 2007). BCMA is virtually absent on naïve and memory B-25 cells (Novak et al., 2004a and b). The BCMA antigen is expressed on the cell surface so is accessible to the antibody, but is also expressed in the golgi. As suggested by its expression profile, BCMA signalling, typically linked with B-cell survival and proliferation, is important in the late stages of B-cell differentiation, as well as the survival of long lived bone marrow plasma cells (O'Connor et al., 2004) and plasmablasts (Avery et al., 2003). Furthermore, as BCMA binds APRIL with high affinity, the BCMA-APRIL signalling axis is suggested to predominate at the later stages of B-cell differentiation, perhaps being the most physiologically relevant interaction.

BCMA expression (both transcript and protein) is reported to correlate with disease progression in various B-cell disorders, including B-cell cancers such as Multiple Myeloma (MM). MM is a clonal B-cell malignancy that occurs in multiple sites within the bone marrow before spreading to the circulation; either de novo, or as a progression from monoclonal gammopathy of undetermined significance (MGUS). It is commonly characterized by increases in paraprotein and osteoclast activity, as well as hypercalcaemia, cytopenia, renal dysfunction, hyperviscosity and peripheral neuropathy. Decreases in both normal antibody levels and numbers of neutrophils are also common, leading to a life threatening susceptibility to infection. BCMA has been implicated in the growth and survival of myeloma cell lines in vitro (Novak et al., 2004 and Moreaux et al., 2004).

### SUMMARY OF THE INVENTION

A composition comprising an isomerized variant of an anti-BCMA antibody, wherein the isomerized variant comprises a heavy chain amino acid sequence comprising a CDRH1 of SEQ ID NO: 1, a CDRH2 of SEQ ID NO: 2, and a CDRH3 of SEQ ID NO: 3, and a light chain amino acid sequence comprising a CDRL1 of SEQ ID NO: 4, a CDRL2 of SEQ ID NO: 5, and a CDRL3 of SEQ ID NO: 6; wherein the composition comprises ≤25% isomerized variant.

A composition comprising an oxidized variant of an anti-BCMA antibody, wherein the oxidized variant comprises a heavy chain amino acid sequence comprising a CDRH1 of SEQ ID NO: 1, a CDRH2 of SEQ ID NO: 2, and a CDRH3 of SEQ ID NO: 3, and a light chain amino acid sequence comprising a CDRL1 of SEQ ID NO: 4, a CDRL2 of SEQ ID NO: 5, and a CDRL3 of SEQ ID NO: 6; wherein the composition comprises ≤40% oxidized variant.

A composition comprising an anti-BCMA antibody comprising a CDRH1 with the amino acid sequence set forth in SEQ ID NO:1, a CDRH2 with the amino acid sequence set forth in SEQ ID NO:2, a CDRH3 with the amino acid sequence set forth in SEQ ID NO:3, a CDRL1 with the amino acid sequence set forth in SEQ ID NO:4, a CDRL2 with the amino acid sequence set forth in SEQ ID NO:5, and a CDRL3 with the amino acid sequence set forth in SEQ ID NO:6; wherein the composition comprises 0.1-25% isomerization at D103 at CDRH3.

A composition comprising an anti-BCMA antibody comprising a CDRH1 with the amino acid sequence set forth in SEQ ID NO:1, a CDRH2 with the amino acid sequence set forth in SEQ ID NO:2, a CDRH3 with the amino acid sequence set forth in SEQ ID NO:3, a CDRL1 with the amino acid sequence set forth in SEQ ID NO:4, a CDRL2 with the amino acid sequence set forth in SEQ ID NO:5, and a CDRL3 with the amino acid sequence set forth in SEQ ID NO:6; wherein the composition comprises 0.1-40% oxidation at M34 at CDRH1.

A composition comprising an anti-BCMA antibody that is at least about 90% identical to the heavy chain amino acid sequence of SEQ ID NO:9 and the light chain amino acid sequence of SEQ ID NO:10, wherein the composition comprises 0.1-25% isomerization at D103 at CDRH3.

A composition comprising an anti-BCMA antibody that is at least about 90% identical to the heavy chain amino acid sequence of SEQ ID NO:9 and the light chain amino acid sequence of SEQ ID NO:10, wherein the composition comprises 0.1-40% oxidation at M34 CDRH1.

A composition comprising an anti-BCMA antibody-drug-conjugate (ADC), wherein the wherein percent DL2 is at least about 30%, about 15% to about 27%, or about 15% to about 32%; percent DL4a is at least about 30%, about 35% to about 38%, or about 30% to about 40%; percent DL4b is at least about 5%, about 7% to about 9%, or about 5% to about 10%; percent DL6 is at least about 10%, about 14% to about 20%, or about 10% to about 20%; and/or DL8 is at least about 1%, about 6.0% to about 12.0%, or about 4% to about 15%.

A composition comprising an anti-BCMA antibody-drug-conjugate (ADC), wherein percent DL0 is less than or equal to about 10% or about 5%.

### BRIEF DESCRIPTION OF THE DRAWINGS

FIG. 1 depicts a schematic representation of a heterogenous mixture of DL species within an ADC composition.

FIG. 2 depicts a representative HIC peak characterization for determining DAR distribution in an ADC composition.

FIG. 3 demonstrates the impact of an average DAR of an ADC composition on tumor volume in a xenograft model.

FIG. 4 depicts a representative cIEF Electropherogram for belantamab.

### DETAILED DESCRIPTION OF THE INVENTION

The invention described herein provides compositions comprising anti-BCMA antigen binding proteins and related methods for treating BCMA-mediated diseases or disorders. It will be understood that a composition comprising anti-BCMA antibodies, as described herein, may also be referred to as a population of anti-BCMA antibodies as described herein: the phrases being interchangeable.

### Anti-BCMA Antigen Binding Proteins

An anti-BCMA antigen binding protein in the compositions described herein may be useful in the treatment or prevention of various BCMA-mediated diseases, including, for example, B-cell mediated cancers such as lymphomas and multiple myeloma. An anti-BCMA antigen binding protein described herein may bind to human BCMA, for example, human BCMA containing the amino acid sequence of GenBank Accession Number Q02223.2, or genes encoding human BCMA having at least 90 percent homology or at least 90 percent identity thereto.

The term “antigen binding protein” as used herein refers to antibodies, antibody fragments and other protein constructs which are capable of binding to BCMA, for example, human BCMA. An antigen binding protein of the present invention may comprise heavy chain variable regions and light chain variable regions of the invention which may be formatted into the structure of a natural antibody or functional fragment or equivalent thereof. An antigen binding protein of the invention may therefore comprise the V_{H} regions of the invention formatted into a full length antibody, a (Fab′)2 fragment, a Fab fragment, or equivalent thereof (such as scFV, bi- tri- or tetra-bodies, Tandabs etc.), when paired with an appropriate light chain. An antibody may be an IgG1, IgG2, IgG3, or IgG4; or IgM; IgA, IgE or IgD or a modified variant thereof. The constant domain of an antibody heavy chain may be selected accordingly. The light chain constant domain may be a kappa or lambda constant domain. Furthermore, an antigen binding protein may comprise modifications of all classes, e.g., IgG dimers, Fc mutants that no longer bind Fc receptors or mediate Clq binding. An antigen binding protein may also be a chimeric antibody of the type described in WO86/01533 which comprises an antigen binding region and a non-immunoglobulin region.

In another aspect of the invention, an antigen binding protein may be either a dAb, Fab, Fab′, F(ab′)2, Fv, diabody, triabody, tetrabody, miniantibody, or a minibody. In one aspect of the present invention, an antigen binding protein may be either a fully human, a humanized, or a chimeric antibody. In a further aspect, an antigen-binding protein is an antibody that is humanized. In one aspect of the invention, an antigen-binding protein is a monoclonal antibody

Chimeric antigen receptors (CARs) have been developed as artificial T cell receptors to generate novel specificities in T cells without the need to bind to MHC-antigenic peptide complexes. These synthetic receptors may contain a target binding domain that is associated with one or more signalling domains via a flexible linker in a single fusion molecule. The target binding domain may be used to target the T cell to specific targets on the surface of pathologic cells and the signalling domains contain molecular machinery for T cell activation and proliferation. The flexible linker which passes through the T cell membrane (i.e. forming a transmembrane domain) may allow for cell membrane display of the target binding domain of the CAR. CARs may successfully allow T cells to be redirected against antigens expressed at the surface of tumour cells from various malignancies including lymphomas and solid tumors (Jena et al., 2010, Blood, 116(7):1035-44). In one aspect of the invention, an anti-BCMA antigen binding protein may comprise a chimeric antigen receptor. In a further aspect, the CAR may comprise a binding domain, a transmembrane domain and an intracellular effector domain.

Exemplary anti-BCMA antigen binding proteins and methods of making the same are disclosed in International Publication No. WO2012/163805 which is incorporated by reference herein in its entirety. Additional exemplary anti-BCMA antigen binding proteins include those described in WO2016/014789, WO2016/090320, WO2016/090327, WO2016/020332, WO2016/079177, WO2014/122143, WO2014/122144, WO2017/021450, WO2016/014565, WO2014/068079, WO2015/166649, WO2015/158671, WO2015/052536, WO2014/140248, WO2013/072415, WO2013/072406, WO2014/089335, US2017/165373, WO2013/154760, and WO2017/051068, each of which is incorporated by reference herein in its entirety.

In another embodiment, an anti-BCMA antigen binding protein described herein may inhibit the binding of BAFF and/or APRIL to the BCMA receptor. In another embodiment, an anti-BCMA antigen binding proteins described herein may be capable of binding to FcγRIIIA or is capable of FcγRIIIA mediated effector function.

In one embodiment, an anti-BCMA antigen binding protein comprises an antibody (“anti-BCMA antibody”). In another embodiment, an anti-BCMA antigen binding protein comprises a monoclonal antibody. The term “antibody” as used herein refers to molecules with an immunoglobulin-like domain (e.g., IgG, IgM, IgA, IgD or IgE) and may include monoclonal, recombinant, polyclonal, chimeric, human, and humanized molecules of this type. Monoclonal antibodies may be produced by a eukaryotic cell clone or a prokaryotic close cell expressing an antibody. Monoclonal antibodies may also be produced by a eukaryotic cell line which can recombinantly express the heavy chain and light chain of the antibody by virtue of having nucleic acid sequences encoding these introduced into the cell. Exemplary methods for producing antibodies from different eukaryotic cell lines such as Chinese Hamster Ovary cells, hybridomas or immortalized antibody cells derived from an animal (e.g., human) are well known to those skilled in the art.

An antibody may be derived, for example, from either rat, mouse, primate (e.g., cynomolgus, Old World monkey or Great Ape), human, or other sources such as nucleic acids generated using molecular biology techniques known to those skilled in the art which encode an antibody molecule.

An antibody may comprise a constant region, which may be of any isotype or subclass. The constant region may be of the IgG isotype, for example, IgG₁, IgG₂, IgG₃, IgG₄ or variants thereof.

An antigen binding protein may comprise one or more modifications including, for example, a mutated constant domain such that, when the antigen binding protein is an antibody, the antibody has enhanced effector functions/ADCC and/or complement activation.

In one embodiment, an anti-BCMA antibody has enhanced antibody dependent cell mediated cytotoxic activity (ADCC) effector function. The term “Effector Function” as used herein is meant to refer to one or more of antibody-dependent cell-mediated cytotoxic activity (ADCC), complement-dependent cytotoxic activity (CDC) mediated responses, Fc-mediated phagocytosis and/or antibody recycling via the FcRn receptor. For IgG antibodies, effector functionalities may include ADCC and ADCP may be mediated by the interaction of the heavy chain constant region with a family of Fcgamma receptors present on the surface of immune cells. In humans these may include FcgammaRI (CD64), FcgammaRII (CD32) and FcgammaRIII (CD16). Interaction between an antigen binding protein bound to antigen and the formation of the Fc/Fcgamma complex may induce a range of effects including cytotoxicity, immune cell activation, phagocytosis and/or release of inflammatory cytokines.

In another embodiment, an anti-BCMA antibody may inhibit the binding of BAFF and/or APRIL to BCMA receptor. In another embodiment, an anti-BCMA antibody may be capable of binding to FcγRIIIA or may be capable of FcγRIIIA mediated effector function. In one embodiment, a composition comprises an anti-BCMA antibody comprising two immunoglobulin (Ig) heavy chains (“HC”) and two Ig light chains (“LC”). The basic antibody structural unit may comprise, for example, a tetramer of subunits. Each tetramer may include two pairs of polypeptide chains, each pair having one “light” (about 25 kDa) and one “heavy” chain (about 50-70 kDa). The amino-terminal portion of each chain may include a variable region of about 100 to 110 or more amino acids primarily responsible for antigen recognition. This variable region may initially be expressed linked to a cleavable signal peptide. The variable region without the signal peptide may be referred to as a mature variable region. Thus, in one example, a light chain mature variable region may comprise a light chain variable region without the light chain signal peptide. The carboxy-terminal portion of each chain may define a constant region. The heavy chain constant region may be primarily responsible for effector function.

The mature variable regions of each light/heavy chain pair may form the antibody binding site (also referred to as the antigen binding site). “Antigen binding site” refers to a site on an antibody which is capable of specifically binding to an antigen, this may be a single variable domain, or it may be paired F_{H}/V_{L} domains as can be found on a standard antibody. Thus, an intact antibody may have, for example, two binding sites. Except in bifunctional or bispecific antibodies, the two binding sites can be the same. The chains all may exhibit the same general structure of relatively conserved framework regions (FR) joined by three hypervariable regions, also called complementarity determining regions or “CDRs”. The CDRs from the two chains of each pair may be aligned by the framework regions, enabling binding to a specific epitope. Thus, in one example, from N-terminal to C-terminal, both light and heavy chains comprise the domains FR1, CDR1, FR2, CDR2, FR3, CDR3 and FR4.

“CDRs” are defined as the complementarity determining region amino acid sequences of an antibody. These are the hypervariable regions of immunoglobulin heavy and light chains. There are three heavy chain and three light chain CDRs (or CDR regions) in the variable portion of an immunoglobulin. Thus, “CDRs” as used herein refers to all three heavy chain CDRs, all three light chain CDRs, all heavy and light chain CDRs, or at least two CDRs. In one embodiment, a composition comprises an anti-BCMA antibody comprising one or more CDR's according to the invention described herein, or one or both of the heavy or light chain variable domains according to the invention described herein.

The terms “variant”, “antibody variant”, “CDR variant” and “post-translational modification variant” refers to at least one amino acid change in an antibody sequence. Variants may be the result of a post translational modification, a chemical change or a sequence change via at least one deletion, substitution or addition. Some post-translational modifications result in a chemical change which does not change the sequence (e.g. Met and oxidized Met; or Asp and isomerized/iso-Asp; or aggregation) while others result in a sequence change such as the conversion of one amino acid residue into another (e.g. Asn conversion to Asp via deamidation; or lysine deletion). Further post-translational modification variants are described below. A variant antibody sequence which comprises a sequence change may be the result of a designed sequence change or a post-translational modification. An amino acid sequence change may be a deletion, substitution or addition.

In one such embodiment, substitutions are conservative substitutions. In an alternative embodiment, an antibody variant comprises at least one substitution whilst retaining the canonical of the antigen binding protein. In one embodiment, an antibody variant is an antibody that is at least about 80%, about 85%, about 90%, or about 95% identical to (i.e. has sequence identity to) the antibody primary sequence. In another embodiment, an antibody variant comprises an antibody comprising a heavy chain amino acid sequence that is at least about 80%, about 85%, about 90%, or about 95% identical to the amino acid sequence of SEQ ID NO:9 and/or a heavy chain amino acid sequence that is at least about 80%, about 85%, about 90%, or about 95% identical to the amino acid sequence of SEQ ID NO:10.

Antigen binding proteins of the present invention may have amino acid modifications that increase the affinity of the constant domain or fragment thereof for FcRn. Increasing the half-life (i.e., serum half-life) of therapeutic and diagnostic IgG antibodies and other bioactive molecules has many benefits including reducing the amount and/or frequency of dosing of these molecules. In one embodiment, an antigen binding protein of the invention comprises all or a portion (an FcRn binding portion) of an IgG constant domain having one or more of the following amino acid modifications.

For example, with reference to IgG1, M252Y/S254T/T256E (commonly referred to as “YTE” mutations) and M428L/N434S (commonly referred to as “LS” mutations) increase FcRn binding at pH 6.0 (Wang et al. 2018).

Half-life can also be enhanced by T250Q/M428L, V2591/V308F/M428L, N434A, and T307A/E380A/N434A mutations (with reference to IgG1 and Kabat numbering) (Monnet et al.).

Half-life and FcRn binding can also be extended by introducing H433K and N434F mutations (commonly referred to as “HN” or “NHance” mutations) (with reference to IgG1) (WO2006/130834).

WO00/42072 discloses a polypeptide comprising a variant Fc region with altered FcRn binding affinity, which polypeptide comprises an amino acid modification at any one or more of amino acid positions 238, 252, 253, 254, 255, 256, 265, 272, 286, 288, 303, 305, 307, 309, 311, 312, 317, 340, 356, 360, 362, 376, 378, 380, 386,388, 400, 413, 415, 424, 433, 434, 435, 436, 439, and 447 of the Fc region (EU index numbering).

WO02/060919 discloses a modified IgG comprising an IgG constant domain comprising one or more amino acid modifications relative to a wild-type IgG constant domain, wherein the modified IgG has an increased half-life compared to the half-life of an IgG having the wild-type IgG constant domain, and wherein the one or more amino acid modifications are at one or more of positions 251, 253, 255, 285-290, 308-314, 385-389, and 428-435.

Shields et al. (2001, J Biol Chem; 276:6591-604) used alanine scanning mutagenesis to alter residues in the Fc region of a human IgG1 antibody and then assessed the binding to human FcRn. Positions that effectively abrogated binding to FcRn when changed to alanine include I253, S254, H435, and Y436. Other positions showed a less pronounced reduction in binding as follows: E233-G236, R255, K288, L309, S415, and H433. Several amino acid positions exhibited an improvement in FcRn binding when changed to alanine; notable among these are P238, T256, E272, V305, T307, Q311, D312, K317, D376, E380, E382, S424, and N434. Many other amino acid positions exhibited a slight improvement (D265, N286, V303, K360, Q362, and A378) or no change (S239, K246, K248, D249, M252, E258, T260, S267, H268, S269, D270, K274, N276, Y278, D280, V282, E283, H285, T289, K290, R292, E293, E294, Q295, Y296, N297, S298, R301, N315, E318, K320, K322, S324, K326, A327, P329, P331, E333, K334, T335, S337, K338, K340, Q342, R344, E345, Q345, Q347, R356, M358, T359, K360, N361, Y373, S375, S383, N384, Q386, E388, N389, N390, K392, L398, S400, D401, K414, R416, Q418, Q419, N421, V422, E430, T437, K439, S440, S442, S444, and K447) in FcRn binding.

The most pronounced effect with respect to improved FcRn binding was found for combination variants. At pH 6.0, the E380A/N434A variant showed over 8-fold better binding to FcRn, relative to native IgG1, compared with 2-fold for E380A and 3.5-fold for N434A. Adding T307A to this resulted in a 12-fold improvement in binding relative to native IgG1. In one embodiment the antigen binding protein of the invention comprises the E380A/N434A mutations and has increased binding to FcRn.

Dall'Acqua et al. (2002, J Immunol.; 169:5171-80) describes random mutagenesis and screening of human IgG1 hinge-Fc fragment phage display libraries against mouse FcRn. They disclosed random mutagenesis of positions 251, 252, 254-256, 308, 309, 311, 312, 314, 385-387, 389, 428, 433, 434, and 436. The major improvements in IgG1-human FcRn complex stability occur when substituting residues located in a band across the Fc-FcRn interface (M252, S254, T256, H433, N434, and Y436) and to lesser extent substitutions of residues at the periphery, such as V308, L309, Q311, G385, Q386, P387, and N389. The variant with the highest affinity to human FcRn was obtained by combining the M252Y/S254T/T256E (“YTE”) and H433K/N434F/Y436H mutations and exhibited a 57-fold increase in affinity relative to the wild-type IgG1. The in vivo behaviour of such a mutated human IgG1 exhibited a nearly 4-fold increase in serum half-life in cynomolgus monkey as compared to wild-type IgG1.

The present invention therefore provides an antigen binding protein with optimized binding to FcRn. In a preferred embodiment, the antigen binding protein comprises at least one amino acid modification in the Fc region of said antigen binding protein, wherein said modification is at an amino acid position selected from the group consisting of 226, 227, 228, 230, 231, 233, 234, 239, 241, 243, 246, 250, 252, 256, 259, 264, 265, 267, 269, 270, 276, 284, 285, 288, 289, 290, 291, 292, 294, 297, 298, 299, 301, 302, 303, 305, 307, 308, 309, 311, 315, 317, 320, 322, 325, 327, 330, 332, 334, 335, 338, 340, 342, 343, 345, 347, 350, 352, 354, 355, 356, 359, 360, 361, 362, 369, 370, 371, 375, 378, 380, 382, 384, 385, 386, 387, 389, 390, 392, 393, 394, 395, 396, 397, 398, 399, 400, 401 403, 404, 408, 411, 412, 414, 415, 416, 418, 419, 420, 421, 422, 424, 426, 428, 433, 434, 438, 439, 440, 443, 444, 445, 446 and 447 of the Fc region.

Additionally, various publications describe methods for obtaining physiologically active molecules with modified half-lives, either by introducing an FcRn-binding polypeptide into the molecules (WO97/43316, U.S. Pat. Nos. 5,869,046, 5,747,035, WO96/32478 and WO91/14438) or by fusing the molecules with antibodies whose FcRn-binding affinities are preserved, but affinities for other Fc receptors have been greatly reduced (WO99/43713), or fusing with FcRn binding domains of antibodies (WO00/09560, U.S. Pat. No. 4,703,039).

FcRn affinity enhanced Fc variants to improve both antibody cytotoxicity and half-life were identified in screens at pH 6.0. The selected IgG variants can be produced as low fucosylated molecules. The resulting variants show increased serum persistence in hFcRn mice, as well as conserved enhanced ADCC (Monnet et al.) Exemplary variants include (with reference to IgG1 and Kabat numbering):

### P230T/V303A/K322R/N389T/F404L/N434S;

### P228R/N434S;

### Q311R/K334R/Q342E/N434Y;

### C226G/Q386R/N434Y;

### T307P/N389T/N434Y;

### P230S/N434S;

### P230T/V305A/T307A/A378V/L398P/N434S;

### P230T/P387S/N434S;

### P230Q/E269D/N434S;

### N276S/A378V/N434S;

### T307A/N315D/A330V/382V/N389T/N434Y;

### T256N/A378V/S383N/N434Y;

### N315D/A330V/N361D/A387V/N434Y;

### V2591/N315D/M428L/N434Y;

### P230S/N315D/M428L/N434Y;

### F241 L/V264E/T307P/A378V/H433R;

### T250A/N389K/N434Y;

### V305A/N315D/A330V/P395A/N434Y;

### V264E/Q386R/P396L/N434S/K439R;

E294del/T307P/N434Y (wherein ‘del’ indicates a deletion).

The present invention also provides a method for the production of an antigen binding protein according to the invention comprising the steps of: a) culturing a recombinant host cell comprising an expression vector comprising the isolated nucleic acid as described herein, wherein the FUT8 gene encoding alpha-1,6-fucosyltransferase has been inactivated in the recombinant host cell; and b) recovering the antigen binding protein. Such methods for the production of antigen binding proteins can be performed, for example, using the POTELLIGENT technology system available from BioWa, Inc. (Princeton, N.J.) in which CHOK1SV cells lacking a functional copy of the FUT8 gene produce monoclonal antibodies having enhanced antibody dependent cell mediated cytotoxicity (ADCC) activity that is increased relative to an identical monoclonal antibody produced in a cell with a functional FUT8 gene. Aspects of the POTELLIGENT technology system are described in U.S. Pat. Nos. 7,214,775, 6,946,292, WO0061739 and WO0231240 all of which are incorporated herein by reference. Those of ordinary skill in the art will also recognize other appropriate systems and methods for generating antigen binding proteins, such as antibodies.

An antibody may be recovered and purified by conventional protein purification procedures. For example, the antibody may be harvested directly from the culture medium. Harvest of the cell culture medium may be via clarification, for example by centrifugation and/or depth filtration. Recovery of the antibody is followed by purification to ensure adequate purity. Therefore, in one aspect, there is provided a cell culture medium comprising an antibody described herein. In one embodiment, the cell culture medium comprises CHO cells.

The antibody may be subsequently purified from the cell culture medium. This may comprise harvesting the cell culture supernatant, placing the cell culture supernatant in contact with a purification medium (e.g. protein A resin or protein G resin to bind antibody molecules) and eluting the antibody molecules from the purification medium to produce an eluate. Therefore, in one aspect, there is provided an eluate comprising an antibody described herein.

One or more chromatography steps may be used in purification, for example one or more chromatography resins; and/or one or more filtration steps. For example, affinity chromatography using resins, such as protein A, G, or L may be used to purify the composition. Alternatively, or in addition to, an ion-exchange resin such as a cation-exchange may be used to purify the composition.

Alternatively, the purification steps comprise: an affinity chromatography resin step, followed by a cation-exchange resin step.

In one embodiment, an anti-BCMA antibody comprises a heavy chain variable region CDR1 (“CDRH1”) comprising an amino acid sequence with at least about 90%, 91%, 92%, 93%, 94%, 95%, 96%, 97%, 98%, 99% or 100% sequence identity to the amino acid sequence set forth in SEQ ID NO:1. In one embodiment, a heavy chain variable region CDR1 (“CDRH1”) comprises an amino acid sequence with one amino acid variation (“variant”) to the amino acid sequence set forth in SEQ ID NO:1.

In one embodiment, an anti-BCMA antibody comprises a heavy chain variable region CDR2 (“CDRH2”) comprising an amino acid sequence with at least about 90%, 91%, 92%, 93%, 94%, 95%, 96%, 97%, 98%, 99% or 100% sequence identity to the amino acid sequence set forth in SEQ ID NO:2. In one embodiment, a heavy chain variable region CDR2 (“CDRH2”) comprises an amino acid sequence with one amino acid variation (“variant”) to the amino acid sequence set forth in SEQ ID NO:2.

In one embodiment, an anti-BCMA antibody comprises a heavy chain variable region CDR3 (“CDRH3”) comprising an amino acid sequence with at least about 90%, 91%, 92%, 93%, 94%, 95%, 96%, 97%, 98%, 99% or 100% sequence identity to the amino acid sequence set forth in SEQ ID NO:3. In one embodiment, a heavy chain variable region CDR3 (“CDRH3”) comprises an amino acid sequence with one amino acid variation (“variant”) to the amino acid sequence set forth in SEQ ID NO:3.

In one embodiment, an anti-BCMA antibody comprises a light chain variable region CDR1 (“CDRL1”) comprising an amino acid sequence with at least about 90%, 91%, 92%, 93%, 94%, 95%, 96%, 97%, 98%, 99% or 100% sequence identity to the amino acid sequence set forth in SEQ ID NO:4. In one embodiment, a light chain variable region CDL1 (“CDR1”) comprises an amino acid sequence with one amino acid variation (“variant”) to the amino acid sequence set forth in SEQ ID NO:4.

In one embodiment, the anti-BCMA antibody comprises a light chain variable region CDR2 (“CDRL2”) comprising an amino acid sequence with at least about 90%, 91%, 92%, 93%, 94%, 95%, 96%, 97%, 98%, 99% or 100% sequence identity to the amino acid sequence set forth in SEQ ID NO:5. In one embodiment, a light chain variable region CDL2 (“CDR2”) comprises an amino acid sequence with one amino acid variation (“variant”) to the amino acid sequence set forth in SEQ ID NO:5.

In one embodiment, the anti-BCMA antibody comprises a light chain variable region CDR3 (“CDRL3”) comprising an amino acid sequence with at least about 90%, 91%, 92%, 93%, 94%, 95%, 96%, 97%, 98%, 99% or 100% sequence identity to the amino acid sequence set forth in SEQ ID NO:6. In one embodiment, a light chain variable region CDL3 (“CDR3”) comprises an amino acid sequence with one amino acid variation (“variant”) to the amino acid sequence set forth in SEQ ID NO:6.

In one embodiment, the anti-BCMA antibody comprises a CDRH1 comprising an amino acid sequence with at least about 90%, 91%, 92%, 93%, 94%, 95%, 96%, 97%, 98%, 99% or 100% sequence identity to the amino acid sequence set forth in SEQ ID NO:1; a CDRH2 comprising an amino acid sequence with at least about 90%, 91%, 92%, 93%, 94%, 95%, 96%, 97%, 98%, 99% or 100% sequence identity to the amino acid sequence set forth in SEQ ID NO:2; a CDRH3 comprising an amino acid sequence with at least about 90%, 91%, 92%, 93%, 94%, 95%, 96%, 97%, 98%, 99% or 100% sequence identity to the amino acid sequence set forth in SEQ ID NO:3; a CDRL1 comprising an amino acid sequence with at least about 90%, 91%, 92%, 93%, 94%, 95%, 96%, 97%, 98%, 99% or 100% sequence identity to the amino acid sequence set forth in SEQ ID NO:4; a CDRL2 comprising an amino acid sequence with at least about 90%, 91%, 92%, 93%, 94%, 95%, 96%, 97%, 98%, 99% or 100% sequence identity to the amino acid sequence set forth in SEQ ID NO:5; and/or a CDRL3 comprising an amino acid sequence with at least about 90%, 91%, 92%, 93%, 94%, 95%, 96%, 97%, 98%, 99% or 100% sequence identity to the amino acid sequence set forth in SEQ ID NO:6.

In one embodiment, the anti-BCMA antibody comprises a heavy chain variable region (“V_{H}”) comprising an amino acid sequence with at least about 90%, 91%, 92%, 93%, 94%, 95%, 96%, 97%, 98%, 99% or 100% sequence identity to the amino acid sequence set forth in SEQ ID NO:7.

In one embodiment, the anti-BCMA antibody comprises a light chain variable region (“V_{L}”) comprising an amino acid sequence with at least about 90%, 91%, 92%, 93%, 94%, 95%, 96%, 97%, 98%, 99% or 100% sequence identity to the amino acid sequence set forth in SEQ ID NO:8.

In one embodiment, the anti-BCMA antibody comprises a V_{H} comprising an amino acid sequence with at least about 90%, 91%, 92%, 93%, 94%, 95%, 96%, 97%, 98%, 99% or 100% sequence identity to the amino acid sequence set forth in SEQ ID NO:7; and a V_{L} comprising an amino acid sequence with at least about 90%, 91%, 92%, 93%, 94%, 95%, 96%, 97%, 98%, 99% or 100% sequence identity to the amino acid sequence set forth in SEQ ID NO:8.

In one embodiment, the anti-BCMA antibody comprises a heavy chain region (“HC”) comprising an amino acid sequence with at least about 90%, 91%, 92%, 93%, 94%, 95%, 96%, 97%, 98%, 99% or 100% sequence identity to the amino acid sequence set forth in SEQ ID NO:9.

In one embodiment, the anti-BCMA antibody comprises a light chain region (“LC”) comprising an amino acid sequence with at least about 90%, 91%, 92%, 93%, 94%, 95%, 96%, 97%, 98%, 99% or 100% sequence identity to the amino acid sequence set forth in SEQ ID NO:10.

In one embodiment, the anti-BCMA antibody comprises a HC comprising an amino acid sequence with at least about 90%, 91%, 92%, 93%, 94%, 95%, 96%, 97%, 98%, 99% or 100% sequence identity to the amino acid sequence set forth in SEQ ID NO:9; and a LC comprising an amino acid sequence with at least about 90%, 91%, 92%, 93%, 94%, 95%, 96%, 97%, 98%, 99% or 100% sequence identity to the amino acid sequence set forth in SEQ ID NO:10.

“Percent identity” between a query amino acid sequence and a subject amino acid sequence is the “Identities” value, expressed as a percentage, that is calculated by the BLASTP algorithm when a subject amino acid sequence has 100% query coverage with a query amino acid sequence after a pair-wise BLASTP alignment is performed. Such pair wise BLASTP alignments between a query amino acid sequence and a subject amino acid sequence are performed by using the default settings of the BLASTP algorithm available on the National Center for Biotechnology Institute's website with the filter for low complexity regions turned off. Importantly, a query sequence may be described by an amino acid sequence identified in one or more claims herein.

In one embodiment, an anti-BCMA antibody comprises a CDRH1 with the amino acid sequence set forth in SEQ ID NO:1; a CDRH2 with the amino acid sequence set forth in SEQ ID NO:2; a CDRH3 with the amino acid sequence set forth in SEQ ID NO:3; a CDRL1 with the amino acid sequence set forth in SEQ ID NO:4; a CDRL2 with the amino acid sequence set forth in SEQ ID NO:5; and a CDRL3 with the amino acid sequence set forth in SEQ ID NO:6.

In one embodiment, an anti-BCMA antibody comprises a V_{H} with the amino acid sequence set forth in SEQ ID NO:7; and a V_{L} with the amino acid sequence set forth in SEQ ID NO:8.

In one embodiment, the anti-BCMA antibody comprises belantamab comprising a HC with the amino acid sequence set forth in SEQ ID NO:9, and a LC with the amino acid sequence set forth in SEQ ID NO:10.

The sequences of antibodies can be determined by the Kabat numbering system (Kabat et al. Sequences of proteins of Immunological Interest NIH, 1987). Alternatively they can be determined using the Chothia numbering system (Al-Lazikani et al., (1997) J M B 273, 927-948), the contact definition method (MacCallum R. M., and Martin A. C. R. and Thornton J. M, (1996), Journal of Molecular Biology, 262 (5), 732-745) or any other established method for numbering the residues in an antibody and determining CDRs known to one skilled in the art. Other numbering conventions for antibody sequences available to a skilled person include “AbM” (University of Bath) and “contact” (University College London) methods. Lastly, antibody sequences can be sequentially numbered.

When numerical reference is made to an amino acid described herein, sequences may be numbered according to the Kabat method or to the sequential numbering method. Unless expressly stated otherwise, numerical reference to a specific amino acid number is described herein with sequential numbering system. Throughout this specification, the terms “CDR”, “CDRL1”, “CDRL2”, “CDRL3”, “CDRH1”, “CDRH2”, “CDRH3” follow Kabat numbering. The amino acid residues in the variable region sequences and full length antibody sequences are numbered sequentially to denote any antibody sequence variant position or post-translational modification variant position, such as an isomerized variant (e.g., D103), a deamidated variant (e.g. N388) or an oxidized variant (e.g., M34).

Reference to a position in the CDR (e.g., M34 or D103) provides the position number in relation to the entire antibody sequence (sequential numbering). Therefore, it will be understood that M34 of CDRH1 refers to the fourth residue of SEQ ID NO: 1, i.e. as underlined: NYWMH (SEQ ID NO: 1). Equally, D103 of CDRH3 refers to the fifth residue of SEQ ID NO: 3, i.e. as underlined: GAIYDGYDVLDN (SEQ ID NO: 3).

In one aspect, a composition comprises an antibody variant comprising a change in one or more amino acids in the primary sequence. In one embodiment, a composition comprises an antibody that is at least about 90% identical to the heavy chain amino acid sequence of SEQ ID NO:9 and/or the light chain sequence of SEQ ID NO:10 with an amino acid change of aspartic acid (D) to asparagine (N), e.g., D103N at CDRH3 D99N in Kabat numbering).

In another embodiment, a composition comprises an antibody comprising a CDRH1 with the amino acid sequence set forth in SEQ ID NO:1, a CDRH2 with the amino acid sequence set forth in SEQ ID NO:2, a CDRH3 with the amino acid sequence set forth in SEQ ID NO:3, a CDRL1 with the amino acid sequence set forth in SEQ ID NO:4, a CDRL2 with the amino acid sequence set forth in SEQ ID NO:5, a CDRL3 with the amino acid sequence set forth in SEQ ID NO:6, and comprises an amino acid change of aspartic acid (D) to asparagine (N), e.g., D103N at CDRH3.

In another embodiment, an anti-BCMA antibody comprises belantamab and comprises an amino acid change of aspartic acid (D) to asparagine (N), e.g., D103N at CDRH3.

In one embodiment, a composition comprises a mixture of antibodies at least about 90% identical to the heavy chain amino acid sequence of SEQ ID NO:9 and/or the light chain sequence of SEQ ID NO:10, wherein about of ≥5%, ≥10%, ≥15%, ≥20%, ≥25%, ≥50%, ≥75%, or ≥90% of the antibody in the mixture comprises D103N at CDRH3.

In one embodiment, a composition comprises a mixture of antibodies comprising a CDRH1 with the amino acid sequence set forth in SEQ ID NO:1, a CDRH2 with the amino acid sequence set forth in SEQ ID NO:2, a CDRH3 with the amino acid sequence set forth in SEQ ID NO:3, a CDRL1 with the amino acid sequence set forth in SEQ ID NO:4, a CDRL2 with the amino acid sequence set forth in SEQ ID NO:5, a CDRL3 with the amino acid sequence set forth in SEQ ID NO:6, wherein about of ≥5%, ≥10%, ≥15%, ≥20%, ≥25%, ≥50%, ≥75%, or ≥90% of the antibody in the mixture comprises D103N at CDRH3.

In one embodiment, a composition comprises belantamab, wherein about of ≥5%, ≥10%, ≥15%, ≥20%, ≥25%, ≥50%, ≥75%, or ≥90% of belantamab comprises D103N at CDRH3.

In one embodiment, the composition comprises belantamab comprising at least one antibody variant using the Kabat numbering system selected from the group consisting of G27Y, 530T, A93T, A24G, K73T, M481, V67A, F71Y, D99N, M4L, and K45E.

### Post-Translational Modification Products

A “post-translational modification product” of an antibody described herein is an antibody composition wherein all or a portion of the composition comprises a “post-translational modification”. Post-translational modifications are chemical changes to the antibody that may be the result from production of the antibody in a host cell, upstream and downstream manufacture, and/or storage (e.g., effect of exposure to light, temperature, pH, water, or by reaction with an excipient and/or the immediate container closure system). Therefore, the composition of the invention may be formed from the manufacture or storage of the antibody. Exemplary post-translational modifications comprise antibody sequence changes (“antibody variant” as described above), cleavage of certain leader sequences, the addition of various sugar moieties in various glycosylation patterns, non-enzymatic glycation, deamidation, oxidation, disulfide bond scrambling and other cysteine variants such as free sulfhydryls, racemized disulfides, thioethers and trisulfide bonds, isomerization, C-terminal lysine cleavage, and/or N-terminal glutamine cyclization.

In one example, a post-translational modification product comprises a “product-related impurity” that comprises a chemical change that results in reduced function and/or activity. In another example, a post-translational modification product comprises a “product-related substance” that comprises a chemical change that does not result in reduced function and/or activity. Product related impurities for the antibodies described herein include isomerized variants and oxidized variants. Product related substances for the antibodies described herein include deamidated variants, glycosylation variants, C-terminal cleaved variants and N-terminal pyro-glutamate variants.

In one embodiment, the composition comprises a heavy chain sequence of SEQ ID NO:9, and a light chain sequence of SEQ ID NO:10, comprising one or more functional post-translational modifications thereof. In another embodiment, the composition comprises a heavy chain sequence of SEQ ID NO:11, SEQ ID NO:12, SEQ ID NO:13, or SEQ ID NO:14, and a light chain of SEQ ID NO:10, comprising one or more functional post-translational modifications thereof.

The percent variant provided herein is expressed as a percentage of the total amount of antibody in the composition (e.g., a “population” of antibodies). For example, 40% or less oxidized variant refers to a total amount of 100% antibody in the composition of which 40% or less is oxidized. For example, 25% or less isomerized variant refers to a total amount of 100% antibody in the composition of which 25% or less is isomerized.

Glycation is a post-translational modification comprising a non-enzymatic chemical reaction between a reducing sugar, such as glucose, and a free amine group in the protein, and is typically observed at the epsilon amine of lysine side chains or at the N-Terminus of the protein. Glycation can occur during production and/or storage in the presence of reducing sugars.

Deamidation, which may, for example, occur during production and/or storage, may be an enzymatic reaction or a chemical reaction. Deamidation may occur via simple chemical reaction through intramolecular cyclization where the amide nitrogen of the next amino acid in the chain nucleophilicly attacks the amide (N+1 attacks N); forming a succinimide intermediate. Deamidation may primarily convert asparagine (N) to iso-aspartic acid (iso-aspartate) and aspartic acid (aspartate) (D) at an approximately 3:1 ratio. This deamidation reaction may therefore be related to isomerization of aspartate (D) to iso-aspartate. The deamidation of asparagine and the isomerization of aspartate, both may involve the intermediate succinimide. To a much lesser degree, deamidation can occur with glutamine residues in a similar manner. Deamidation can occur in a CDR, in a Fab (non-CDR region), or in an Fc region. Isomerization is the conversion of aspartate (D) to iso-aspartate which involves the intermediate succinimide.

Oxidation can occur during production and/or storage (i.e. in the presence of oxidizing conditions) and results in a covalent modification of a protein, induced either directly by reactive oxygen species or indirectly by reaction with secondary by-products of oxidative stress. Oxidation may happen primarily with methionine residues, but may also occur at tryptophan and free cysteine residues. Oxidation can occur in a CDR, in a Fab (non-CDR) region, or in an Fc region.

Disulfide bond scrambling can occur during production and/or storage conditions. Under certain circumstances, disulfide bonds may break or form incorrectly, resulting in unpaired cysteine residues (—SH). These free (unpaired) sulfhydryls (—SH) may promote shuffling.

The formation of a thioether and racemization of a disulphide bond can occur under basic conditions, in production or storage, through a beta elimination of di-sulphide bridges back to cysteine residues via a dehydroalanine and persulfide intermediate. Subsequent crosslinking of dehydroalanine and cysteine may result in the formation of a thioether bond or the free cysteine residues may reform a disulphide bond with a mixture of D- and L-cysteine.

Trisulfides may result from insertion of a sulfur atom into a disulphide bond (Cys-S-S-S-Cys) and may be formed due to the presence of hydrogen sulphide in production cell culture.

N-terminal glutamine (Q) and glutamate (glutamic acid) (E) in the heavy chain and/or light chain may form pyroglutamate (pGlu) via cyclization. pGlu formation may form in the production bioreactor, but it can also be formed, for example, non-enzymatically, depending on pH and temperature of processing and storage conditions. Cyclization of N-terminal Q or E is commonly observed in natural human antibodies.

C-terminal lysine cleavage is an enzymatic reaction catalyzed by carboxypeptidases, and is commonly observed in recombinant and natural human antibodies. Variants of this process include removal of lysine from one or both heavy chains due to cellular enzymes from the recombinant host cell. Administration to the human subject/patient is likely to result in the removal of any remaining C-terminal lysine.

The present invention encompasses antibodies which may have been subjected to, or have undergone, one or more of a post-translational modification described herein. Exemplary compositions may comprise a mixture or blend of antibodies: 1) with and without post-translational modifications (1 or more), or 2) with more than one type of a post-translational modifications described herein.

The composition may comprise a mixture of antibody variants and post-translational modification variants. For example, the antibody composition may comprise one or more, such as two or more of oxidation variants, deamidation variants, isomerized variants, N-terminal pyro-glutamate variants, and C-terminal lysine cleaved variants.

For example, in one embodiment, a composition may comprise a mixture of antibodies, wherein 10% of the antibody in the mixture comprises the amino acid sequence of SEQ ID NO 9 and 10, and 90% of the antibody in the mixture comprises the amino acid sequence of SEQ ID NO 9 and 10 with a C-terminal lysine cleavage.

In another exemplary embodiment, a composition may comprise a mixture of antibodies, wherein 10% of the antibody in the mixture comprises the amino acid sequence of SEQ ID NO 9 and 10, 90% of the antibody in the mixture comprises the amino acid sequence of SEQ ID NO 9 and 10 with a C-terminal lysine cleavage, and of that 100% total antibody mixture, up to 100% of the N-terminal glutamine is cyclized to pyro-glutamate.

In another exemplary embodiment, a composition may comprise a mixture of antibodies, wherein 10% of the antibody in the mixture comprises the amino acid sequence of SEQ ID NO 9 and 10, 90% of the antibody in the mixture comprises the amino acid sequence of SEQ ID NO 9 and 10 with a C-terminal lysine cleavage, and of that 100% total antibody mixture, up to 100% is N-terminal pyro-glutamate and up to 23% is isomerized at D103 at CDRH3.

In yet another exemplary embodiment, a composition comprises a mixture of antibodies, wherein 20% of the antibody in the mixture comprises the amino acid sequence of SEQ ID NO 9 and 10, 80% of the antibody in the mixture comprises the amino acid sequence of SEQ ID NO 9 and 10 with variant N103 at CDRH3, and of that 100% total antibody mixture, up to 37% of the antibody is oxidized at amino acid M34 CDRH1.

In one embodiment, a post-translational modification described herein, does not result in a significant change in antigen binding affinity, biological activity, pharmacokinetics (PK)/pharmacodynamics (PD), aggregation, immunogenicity, and/or binding to an Fc receptor, except where specified and described as a product-related impurity.

“Function” or “activity” as described herein is defined as one or more of 1) binding to BCMA, 2) binding to FcγRIIIa, and/or 3) binding to FcRn. In one embodiment, “reduced function” or “reduced activity” means that binding to BCMA, binding to FcγRIIIa, or binding to FcRn is reduced as a percentage compared to a reference standard, and is significant over assay variability. For example, reduced function or activity can be described as a reduction of ≥5%, ≥10%, ≥15%, ≥20%, ≥25%, ≥30%, ≥35%, ≥40%, ≥45%, or ≥50%.

In one embodiment, an anti-BCMA antibody comprises an antibody that is at least about 90% identical to the amino acid sequences of SEQ ID NO:9 and SEQ ID NO:10 and includes all post-translational modifications, if any, of the antibody.

In another embodiment, an anti-BCMA antibody comprises belantamab and all post-translational modifications if any.

Antibody variants are commonly observed when the composition of antibodies is analyzed by charged based-separation techniques such as isoelectric focusing (IEF) gel electrophoresis, capillary isoelectric focusing (cIEF) gel electrophoresis, cation exchange chromatography (CEX) and anion exchange chromatography (AEX).

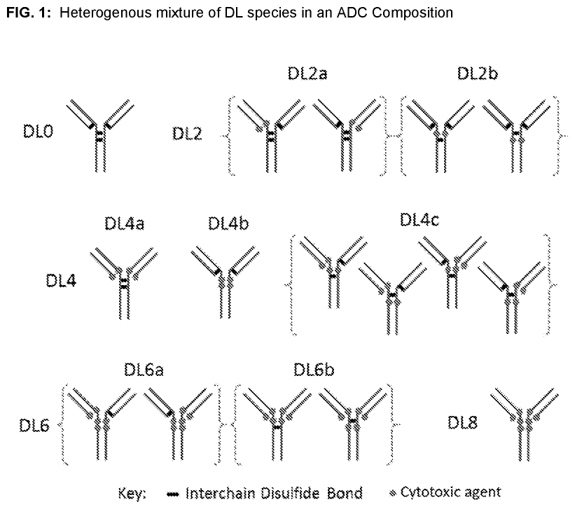

Post translational modifications can result in an increase or decrease in the net charge of the antibody and cause a decrease or increase in the pl value, thereby leading to acidic variants and basic variants (collectively called “charged variants”) with respect to the main isoform. The main isoform is the antibody population that elutes as the major peak on chromatograms. Acidic species are variants with lower apparent pl and basic species are variants with higher apparent pl, when antibodies are analyzed using IEF based methods. When analyzed by chromatography-based methods, acidic species and basic species are defined based on their retention times relative to the main peak. Acidic species are the variants that elute earlier than the main peak from CEX or later then than the main peak from AEX, while basic species are the variants that elute later than the main peak from CEX or earlier than the main peak from AEX. These methods separate the main isoform of the antibody from the acidic isoform (acidic variant) and basic isoform (basic variant). The charged variant can be detected by various methods, such as ion exchange chromatography, for example, WCX-10 HPLC (a weak cation exchange chromatography) or IEF (isoelectric focusing). The percent charged variant can be determined using capillary isoelectric focusing (cIEF). Capillary isoelectric focusing (cIEF) was used to measure the pl of dostarlimab and separate charge variants (see FIG. 1). The method can be used to quantitate the acidic and basic species as a percentage of the total area peak. The terms “species”, “isoform”, “form” and “peak” are used interchangeably to refer to the main isoform and the charged variant (acidic variant and basic variant).

In one embodiment, the composition comprises an acidic variant of the antibody, wherein the acidic variant comprises a CDRH1 of SEQ ID NO: 1, a CDRH2 of SEQ ID NO: 2, and a CDRH3 of SEQ ID NO: 3, and a light chain amino acid sequence comprising a CDRL1 of SEQ ID NO: 4, a CDRL2 of SEQ ID NO: 5, and a CDRL3 of SEQ ID NO: 6; wherein the composition comprises 1-70% acidic variant.

In one aspect, the composition comprises ≤70%, acidic variant. In one embodiment, the composition comprises ≤60%, ≤50%, ≤40%, ≤35%, or ≤30%, acidic variant. Alternatively, the composition comprises 10-70%, 10-60%, 10-50%, 10-40%, 10-35%, or 10-30% acidic variant. Alternatively, the composition comprises 20-70%, 20-60%, 20-50%, 20-40%, 20-35%, or 20-30% acidic variant. Alternatively, the composition comprises about 60%, about 50%, about 40%, about 35%, about 30%, about 25%, or about 20% acidic variant.

In one aspect, the composition comprises a basic variant of the antibody, wherein the basic variant comprises a heavy chain amino acid sequence comprising a CDRH1 of SEQ ID NO: 1, a CDRH2 of SEQ ID NO: 2, and a CDRH3 of SEQ ID NO: 3, and a light chain amino acid sequence comprising a CDRL1 of SEQ ID NO: 4, a CDRL2 of SEQ ID NO: 5, and a CDRL3 of SEQ ID NO: 6; wherein the composition comprises 1-30% basic variant.

In one aspect, the composition comprises ≤30%, basic variant. In one embodiment, the composition comprises ≤25%, ≤20%, ≤15%, ≤10%, ≤7.5%, or ≤5%, basic variant. In one embodiment, the composition comprises 1-30%, 1-25%, 1-20%, 1-15%, 1-10%, or 1-5% basic variant. Alternatively, the composition comprises about 15%, about 10%, or about 5% basic variant.

In one aspect, the composition comprises a main isoform of the antibody, wherein the main isoform comprises a heavy chain amino acid sequence comprising a CDRH1 of SEQ ID NO: 1, a CDRH2 of SEQ ID NO: 2, and a CDRH3 of SEQ ID NO: 3, and a light chain amino acid sequence comprising a CDRL1 of SEQ ID NO: 4, a CDRL2 of SEQ ID NO: 5, and a CDRL3 of SEQ ID NO: 6; wherein the composition comprises 1-90% main isoform.

In one aspect, the composition comprises ≥1%, main isoform. In one embodiment, the composition comprises ≥5%, ≥10%, ≥20%, ≥30%, ≥40%, ≥50%, ≥55%, ≥60%, ≥65%, ≥70%, ≥75%, ≥80% or ≥90% main isoform. In one embodiment, the composition comprises 10-90%, 20-90%, 30-90%, 40-90%, 50-90% or 60-90% main isoform. In one embodiment, the composition comprises 10-80%, 20-80%, 30-80%, 40-80%, 50-80% or 60-80% main isoform. Alternatively, the composition comprises about 80%, about 75%, about 70%, about 65%, about 60%, about 50% or about 55% main isoform.

The percent acidic variant, percent basic variant and percent main isoform can be determined using capillary isoelectric focusing (cIEF). It will be understood that these isoform/charged variant embodiments may be combined with any one or a combination of the antibody variants described herein.

In one aspect, the composition comprises a charged variant of the antibody comprising a heavy chain amino acid sequence comprising a CDRH1 of SEQ ID NO: 1, a CDRH2 of SEQ ID NO: 2, and a CDRH3 of SEQ ID NO: 3, and a light chain amino acid sequence comprising a CDRL1 of SEQ ID NO: 4, a CDRL2 of SEQ ID NO: 5, and a CDRL3 of SEQ ID NO: 6; wherein the composition comprises: ≤70%, acidic variant; and/or ≤30%, basic variant; and/or ≥1%, main isoform.

In one aspect, a composition comprises an antibody comprising an isomerization post-translational modification (“isomerization” or “isomerized”) or an “isomerized variant”. The variant may comprise an isomerized amino acid residue in the heavy chain sequence and/or the light chain sequence, such as a CDR of the heavy chain sequence and/or a CDR of the light chain sequence. The isomerized variant may be present in one or both chains of the heavy chain or light chain. An isomerization post-translational modification results in iso-aspartate and/or succinimide-aspartate. In one example, aspartic acid (Asp) isomerization can be determined using tryptic peptide mapping tandem mass spectrometry (peptide mapping LC-MS/MS) as described herein. It will be understood that these isomerized variant embodiments may be combined with the antibody features described herein.

In one embodiment, the composition comprises an isomerized variant of an anti-BCMA antibody, wherein the isomerized variant comprises a heavy chain amino acid sequence comprising a CDRH1 of SEQ ID NO: 1, a CDRH2 of SEQ ID NO: 2, and a CDRH3 of SEQ ID NO: 3, and a light chain amino acid sequence comprising a CDRL1 of SEQ ID NO: 4, a CDRL2 of SEQ ID NO: 5, and a CDRL3 of SEQ ID NO: 6; wherein the composition comprises ≤25% isomerized variant.

In one aspect, the composition comprises a population of anti-BCMA antibodies that includes:
antibodies comprising a heavy chain amino acid sequence comprising SEQ ID NO: 1 (CDRH1), SEQ ID NO: 2 (CDRH2), and SEQ ID NO: 3 (CDRH3) and a light chain amino acid sequence comprising SEQ ID NO: 4 (CDRL1), SEQ ID NO: 5 (CDRL2) and SEQ ID NO: 6 (CDRL3), and
isomerized variants thereof, wherein ≤25% of the population of antibodies is comprised of the isomerized variants.

In another embodiment, the composition comprises an isomerized variant of an anti-BCMA antibody, wherein the isomerized variant comprises a heavy chain amino acid sequence comprising a CDRH1 of SEQ ID NO: 1, a CDRH2 of SEQ ID NO: 2, and a CDRH3 of SEQ ID NO: 3, and a light chain amino acid sequence comprising a CDRL1 of SEQ ID NO: 4, a CDRL2 of SEQ ID NO: 5, and a CDRL3 of SEQ ID NO: 6; wherein the composition comprises ≤25% isomerized variant at amino acid D103 in CDRH3.

In one embodiment, the composition comprises an isomerized variant of an anti-BCMA antibody, wherein the isomerized variant comprises a heavy chain sequence of SEQ ID NO: 9 and a light chain sequence of SEQ ID NO: 10; wherein the composition comprises ≤25% isomerized variant.

Alternatively, the isomerized variant comprises a heavy chain sequence of SEQ ID NO: 11, 12, 13 or 14.

In one embodiment, the composition comprises an antibody that is at least about 90% identical to the heavy chain amino acid sequence of SEQ ID NO:9 and/or the light chain sequence of SEQ ID NO:10, and comprises isomerization in either the heavy chain sequence or light chain sequence, e.g., isomerization at amino acid D103 at CDRH3.

In another embodiment, a composition comprises an antibody comprising a CDRH1 with the amino acid sequence set forth in SEQ ID NO:1, a CDRH2 with the amino acid sequence set forth in SEQ ID NO:2, a CDRH3 with the amino acid sequence set forth in SEQ ID NO:3, a CDRL1 with the amino acid sequence set forth in SEQ ID NO:4, a CDRL2 with the amino acid sequence set forth in SEQ ID NO:5, a CDRL3 with the amino acid sequence set forth in SEQ ID NO:6, and isomerization in at least one of the six CDR regions, e.g., isomerization at amino acid D103 at CDRH3.

In another embodiment, an anti-BCMA antibody comprises belantamab and comprises isomerization in either the heavy chain sequence or light chain sequence, e.g., isomerization at amino acid D103 at CDRH3.

In one embodiment, a composition comprises a mixture of antibodies at least about 90% identical to the heavy chain amino acid sequence of SEQ ID NO:9 and/or the light chain sequence of SEQ ID NO:10, wherein about ≤25%, ≤23%, ≤20%, ≤15%, ≤10%, ≤8%, ≤7%, 0.1-25%, 0.1-20%, 0.1-15%, 0.1-10%, 0.1-8%, 0.1-7%, 1-6%, 2-6%, 3-6%, about 4% about 5% or about 6% of the antibody in the mixture is isomerized at amino acid D103 at CDRH3. In one embodiment, a composition comprising ≤25% or ≤23%, isomerization at D103 at CDRH3 retains ≥70%, BCMA specific antigen binding.

In one embodiment, a composition comprises a mixture of antibodies comprising a CDRH1 with the amino acid sequence set forth in SEQ ID NO:1, a CDRH2 with the amino acid sequence set forth in SEQ ID NO:2, a CDRH3 with the amino acid sequence set forth in SEQ ID NO:3, a CDRL1 with the amino acid sequence set forth in SEQ ID NO:4, a CDRL2 with the amino acid sequence set forth in SEQ ID NO:5, a CDRL3 with the amino acid sequence set forth in SEQ ID NO:6, wherein about ≤25%, ≤23%, ≤20%, ≤15%, ≤10%, ≤8%, ≥7%, 0.1-25%, 0.1-20%, 0.1-15%, 0.1-10%, 0.1-8%, 0.1-7%, 1-6%, 2-6%, 3-6%, about 4% about 5% or about 6% of the antibody in the mixture is isomerized at amino acid D103 at CDRH3. In one embodiment, a composition comprising ≤25% or ≤23%, isomerization at D103 at CDRH3 retains ≥70%, BCMA specific antigen binding.

In another embodiment, a composition comprises belantamab, wherein about ≤25%, ≤23%, ≤20%, ≤15%, ≤10%, ≤8%, ≤7%, 0.1-25%, 0.1-20%, 0.1-15%, 0.1-10%, 0.1-8%, 0.1-7%, 1-6%, 2-6%, 3-6%, about 4% about 5% or about 6% of belantamab is isomerized at amino acid D103 at CDRH3. In one embodiment, belantamab comprising ≤25% or ≤23%, isomerization at D103 at CDRH3 retains ≥70%, BCMA specific antigen binding.

In one embodiment, the composition comprises an isomerized variant of an anti-BCMA antibody, wherein the isomerized variant comprises a heavy chain sequence of SEQ ID NO: 9 and a light chain sequence of SEQ ID NO: 10; wherein the composition comprises ≤25% isomerized variant.

In one example, aspartic acid (Asp) isomerization can be determined using tryptic peptide mapping tandem mass spectrometry (peptide mapping LC-MS/MS). In one example, a sample comprising a composition described herein may be denatured e.g., in 6 M guanidine HCl to a concentration of e.g., 4.2 μg/μL. The disulfide bonds may then be reduced e.g., with 50 mM DTT for 20 minutes at room temperature. lodoacetate, e.g., can then be added at e.g., 100 mM and reacted with the free cysteine residues e.g., for 30 minutes at room temperature, protected from light. The sample can then be buffer exchanged e.g., using a BioRad spin columns (part no. 7326221), before digestion e.g., with 0.5% trypsin at for 15 minutes at 37° C. The resulting peptides can then be loaded onto a reversed phase ultra-performance liquid chromatography (UPLC) column and can be eluted with a water and acetonitrile gradient in e.g., 0.1% trifluoroacetic acid using a UPLC. The peptides can then be detected with a UV detector and a mass spectrometer, (e.g., Thermo Scientific LTQ Orbitrap XL). The extracted ion chromatograms of the unmodified and modified peptides can be used to calculate the levels of isomerization by dividing the area under the curve of the modified peptide by the total areas under the curve for both modified and unmodified peptides.

In one aspect, a composition comprises an antibody comprising an oxidation post-translational modification (“oxidation” or “oxidized”) or an “oxidized variant”. The variant may comprise an oxidized amino acid residue in the heavy chain sequence and/or the light chain sequence, such as a CDR of the heavy chain sequence and/or a CDR of the light chain sequence. The oxidized variant may be present in one or both chains of the heavy chain or light chain. It will be understood that these oxidized variant embodiments may be combined with the antibody features described herein.

In one embodiment, the composition comprises an oxidized variant of an anti-BCMA antibody, wherein the oxidized variant comprises a heavy chain amino acid sequence comprising a CDRH1 of SEQ ID NO: 1, a CDRH2 of SEQ ID NO: 2, and a CDRH3 of SEQ ID NO: 3, and a light chain amino acid sequence comprising a CDRL1 of SEQ ID NO: 4, a CDRL2 of SEQ ID NO: 5, and a CDRL3 of SEQ ID NO: 6; wherein the composition comprises ≤40% oxidized variant.

In one aspect, the composition comprises a population of anti-BCMA antibodies that includes:
antibodies comprising a heavy chain amino acid sequence comprising SEQ ID NO: 1 (CDRH1), SEQ ID NO: 2 (CDRH2), and SEQ ID NO: 3 (CDRH3) and a light chain amino acid sequence comprising SEQ ID NO: 4 (CDRL1), SEQ ID NO: 5 (CDRL2) and SEQ ID NO: 6 (CDRL3), and
oxidized variants thereof, wherein 40% of the population of antibodies is comprised of the oxidized variants.

In one embodiment, the oxidized variant comprises oxidation in one or more of the CDRs. In a further embodiment, the oxidized variant comprises oxidation at a methionine and/or tryptophan residue in any one of SEQ ID NOs: 1-6.

In another embodiment, the composition comprises an oxidized variant of an anti-BCMA antibody, wherein the oxidized variant comprises a heavy chain amino acid sequence comprising a CDRH1 of SEQ ID NO: 1, a CDRH2 of SEQ ID NO: 2, and a CDRH3 of SEQ ID NO: 3, and a light chain amino acid sequence comprising a CDRL1 of SEQ ID NO: 4, a CDRL2 of SEQ ID NO: 5, and a CDRL3 of SEQ ID NO: 6; wherein the composition comprises ≤40% oxidized variant at amino acid M34 in CDRH1.

In one embodiment, the composition comprises an oxidized variant of belantamab, wherein the oxidized variant comprises a heavy chain sequence of SEQ ID NO: 9 and a light chain sequence of SEQ ID NO: 10; wherein the composition comprises ≤40% oxidized variant.

In one embodiment, the composition comprises an antibody that is at least about 90% identical to the heavy chain amino acid sequence of SEQ ID NO:9 and/or the light chain sequence of SEQ ID NO:10, and comprises oxidation in the heavy chain sequence, e.g., oxidation at amino acid M34 (CDRH1), M256 and/or M432.

In another embodiment, a composition comprises an antibody comprising a CDRH1 with the amino acid sequence set forth in SEQ ID NO:1, a CDRH2 with the amino acid sequence set forth in SEQ ID NO:2, a CDRH3 with the amino acid sequence set forth in SEQ ID NO:3, a CDRL1 with the amino acid sequence set forth in SEQ ID NO:4, a CDRL2 with the amino acid sequence set forth in SEQ ID NO:5, a CDRL3 with the amino acid sequence set forth in SEQ ID NO:6, and comprises oxidation in the heavy chain sequence, e.g., oxidation at amino acid M34 (CDRH1), M256 and/or M432.

In another embodiment, an anti-BCMA antibody comprises belantamab and comprises oxidation in the heavy chain sequence, e.g., oxidation at amino acid M34 (CDRH1), M256 and/or M432.

In one embodiment, a composition comprises a mixture of antibodies at least about 90% identical to the heavy chain amino acid sequence of SEQ ID NO:9 and/or the light chain sequence of SEQ ID NO:10, wherein about ≤40%, ≤35%, ≤30%, ≤25%, ≤20%, ≤15%, ≤10%, ≤7.5%, ≤5%, ≤2.5%, ≤2%, 0.1-40%, 0.1-35%, 0.1-30%, 0.1-25%, 0.1-20%, 0.1-15%, 0.1-10%, 0.1-7.5%, 0.1-5%, 0.1-2.5%, 0.1-2%, about 0.5%, about 1%, about 2%, or about 5% of the antibody in the mixture is oxidized at amino acid M34. In one embodiment, a composition comprising 40% oxidation at heavy chain M34 retains ≥70%, BCMA specific antigen binding. In another embodiment, a composition comprising ≤37%, oxidation at heavy chain M34 retains ≥70%, BCMA specific antigen binding.

In one embodiment, a composition comprises a mixture of antibodies comprising a CDRH1 with the amino acid sequence set forth in SEQ ID NO:1, a CDRH2 with the amino acid sequence set forth in SEQ ID NO:2, a CDRH3 with the amino acid sequence set forth in SEQ ID NO:3, a CDRL1 with the amino acid sequence set forth in SEQ ID NO:4, a CDRL2 with the amino acid sequence set forth in SEQ ID NO:5, a CDRL3 with the amino acid sequence set forth in SEQ ID NO:6, wherein about ≤40%, ≤35%, 30%, ≤25%, ≤20%, ≤15%, ≤10%, ≤7.5%, ≤5%, ≤2.5%, ≤2%, 0.1-40%, 0.1-35%, 0.1-30%, 0.1-25%, 0.1-20%, 0.1-15%, 0.1-10%, 0.1-7.5%, 0.1-5%, 0.1-2.5%, 0.1-2%, about 0.5%, about 1%, about 2%, or about 5% of the antibody in the mixture is oxidized at amino acid M34. In one embodiment, a composition comprising 40% oxidation at heavy chain M34 retains ≥70%, BCMA specific antigen binding. In another embodiment, a composition comprising ≤37%, oxidation at heavy chain M34 retains ≥70%, BCMA specific antigen binding.

In another embodiment, a composition comprises belantamab, wherein about ≤40%, ≤35%, 30%, ≤25%, ≤20%, ≤15%, ≤10%, ≤7.5%, ≤5%, ≤2.5%, ≤2%, 0.1-40%, 0.1-35%, 0.1-30%, 0.1-25%, 0.1-20%, 0.1-15%, 0.1-10%, 0.1-7.5%, 0.1-5%, 0.1-2.5%, 0.1-2%, about 0.5%, about 1%, about 2%, or about 5% of belantamab is oxidized at amino acid M34. In one embodiment, belantamab comprising ≤40% oxidation at heavy chain M34 retains ≥70%, BCMA specific antigen binding. In another embodiment, belantamab comprising ≤37% oxidation at heavy chain M34 retains ≥70%, BCMA specific antigen binding.

In one embodiment, the composition comprises an oxidized variant of an anti-BCMA antibody, wherein the oxidized variant comprises a heavy chain amino acid sequence comprising a CDRH1 of SEQ ID NO: 1, a CDRH2 of SEQ ID NO: 2, and a CDRH3 of SEQ ID NO: 3, and a light chain amino acid sequence comprising a CDRL1 of SEQ ID NO: 4, a CDRL2 of SEQ ID NO: 5, and a CDRL3 of SEQ ID NO: 6; wherein the composition comprises ≤90%, oxidized variant in the Fc region.

In one embodiment, the antibody comprises oxidation at a methionine and/or tryptophan residue in the Fc region of the heavy chain sequence and/or the Fc region of the light chain sequence. In some embodiments, the oxidized variant comprises one or a combination of oxidation at: M256 and/or M432 of the Fc region of the heavy chain sequence.

In another embodiment, the composition comprises an oxidized variant of an anti-BCMA antibody, wherein the oxidized variant comprises a heavy chain amino acid sequence comprising a CDRH1 of SEQ ID NO: 1, a CDRH2 of SEQ ID NO: 2, and a CDRH3 of SEQ ID NO: 3, and a light chain amino acid sequence comprising a CDRL1 of SEQ ID NO: 4, a CDRL2 of SEQ ID NO: 5, and a CDRL3 of SEQ ID NO: 6; wherein the composition comprises ≤90%, oxidized M256 and/or M432 variant.

In one embodiment, the composition comprises an oxidized variant of belantamab, wherein the oxidized variant comprises a heavy chain sequence of SEQ ID NO: 9 and a light chain sequence of SEQ ID NO: 10; wherein the composition comprises ≤90%, oxidized variant in the Fc region.

Alternatively, the oxidized variant comprises a heavy chain sequence of SEQ ID NO: 11, 12, 13 or 14.

In one embodiment, a composition comprises a mixture of antibodies at least about 90% identical to the heavy chain amino acid sequence of SEQ ID NO:9 and/or the light chain sequence of SEQ ID NO:10, wherein about ≤90%, ≤80%, ≤70%, ≤65%, ≤50%, ≤40%, ≤30%, ≤20%, ≤10%, ≤7.5%, ≤5%, 0.1-90%, 0.1-80%, 0.1-70%, 0.1-65%, 0.1-50%, 0.1-40%, 0.1-30%, 0.1-20%, 0.1-10%, 1-10%, 1-5%, 2-10%, 2-4%, about 2%, about 3%, or about 4% of the antibody in the mixture is oxidized at amino acid M256. In one embodiment, a composition comprising ≤90%, or ≤89% oxidation at heavy chain M256 retains ≥70%, FcγRIIIA binding. In another embodiment, a composition comprising ≤65%, or ≤64%, oxidation at heavy chain M256 retains ≥70%, FcRn binding.

In one embodiment, a composition comprises a mixture of antibodies comprising a CDRH1 with the amino acid sequence set forth in SEQ ID NO:1, a CDRH2 with the amino acid sequence set forth in SEQ ID NO:2, a CDRH3 with the amino acid sequence set forth in SEQ ID NO:3, a CDRL1 with the amino acid sequence set forth in SEQ ID NO:4, a CDRL2 with the amino acid sequence set forth in SEQ ID NO:5, a CDRL3 with the amino acid sequence set forth in SEQ ID NO:6, wherein about ≤90%, ≤80%, ≤70%, ≤65%, ≤50%, ≤40%, ≤30%, ≤20%, ≤10%, ≤7.5%, ≤5%, 0.1-90%, 0.1-80%, 0.1-70%, 0.1-65%, 0.1-50%, 0.1-40%, 0.1-30%, 0.1-20%, 0.1-10%, 1-10%, 1-5%, 2-10%, 2-4%, about 2%, about 3%, or about 4% of the antibody in the mixture is oxidized at amino acid M256. In one embodiment, a composition comprising ≤90%, or ≤89% oxidation at heavy chain M256 retains ≥70%, FcγRIIIA binding. In another embodiment, a composition comprising ≤65%, or ≤64%, oxidation at heavy chain M256 retains ≥70%, FcRn binding.

In another embodiment, a composition comprises belantamab, wherein about ≤90%, ≤80%, ≤70%, ≤65%, ≤50%, ≤40%, ≤30%, ≤20%, ≤10%, ≤7.5%, ≤5%, 0.1-90%, 0.1-80%, 0.1-70%, 0.1-65%, 0.1-50%, 0.1-40%, 0.1-30%, 0.1-20%, 0.1-10%, 1-10%, 1-5%, 2-10%, 2-4%, about 2%, about 3%, or about 4% of belantamab is oxidized at amino acid M256. In one embodiment, belantamab comprising ≤90%, or ≤89% oxidation at heavy chain M256 retains ≥70%, FcγRIIIA binding. In another embodiment, belantamab comprising ≤65%, or ≤64%, oxidation at heavy chain M256 retains ≥70%, FcRn binding.

In one embodiment, a composition comprises a mixture of antibodies at least about 90% identical to the heavy chain amino acid sequence of SEQ ID NO:9 and/or the light chain sequence of SEQ ID NO:10, wherein about ≤86%, ≤70%, ≤60%, ≤50%, ≤40%, ≤30%, ≤20%, ≤10%, ≤7.5%, ≤5%, ≤2.5%, ≤2%, 0.1-86%, 0.1-70%, 0.1-60%, 0.1-50%, 0.1-40%, 0.1-30%, 0.1-20%, 0.1-10%, 0.1-5%, 0.1-3%, about 0.5%, about 1%, about 2%, or about 3% of the antibody in the mixture is oxidized at amino acid M432. In one embodiment, a composition comprising ≤86%, oxidation at heavy chain M432 retains ≥70%, FcγRIIA binding. In another embodiment, a composition comprising ≤60%, oxidation at heavy chain M432 retains ≥70%, FcRn binding.

In one embodiment, a composition comprises a mixture of antibodies comprising a CDRH1 with the amino acid sequence set forth in SEQ ID NO:1, a CDRH2 with the amino acid sequence set forth in SEQ ID NO:2, a CDRH3 with the amino acid sequence set forth in SEQ ID NO:3, a CDRL1 with the amino acid sequence set forth in SEQ ID NO:4, a CDRL2 with the amino acid sequence set forth in SEQ ID NO:5, a CDRL3 with the amino acid sequence set forth in SEQ ID NO:6, wherein about ≤86%, ≤70%, ≤60%, ≤50%, ≤40%, ≤30%, ≤20%, ≤10%, ≤7.5%, ≤5%, ≤2.5%, ≤2%, 0.1-86%, 0.1-70%, 0.1-60%, 0.1-50%, 0.1-40%, 0.1-30%, 0.1-20%, 0.1-10%, 0.1-5%, 0.1-3%, about 0.5%, about 1%, about 2%, or about 3% of the antibody in the mixture is oxidized at amino acid M432. composition comprising ≤86%, oxidation at heavy chain M432 retains ≥70%, FcγRIIIA binding. In another embodiment, a composition comprising ≤60%, oxidation at heavy chain M432 retains ≥70%, FcRn binding.

In another embodiment, a composition comprises belantamab, wherein about ≤86%, ≤70%, ≤60%, ≤50%, ≤40%, ≤30%, ≤20%, ≤10%, ≤7.5%, ≤5%, ≤2.5%, ≤2%, 0.1-86%, 0.1-70%, 0.1-60%, 0.1-50%, 0.1-40%, 0.1-30%, 0.1-20%, 0.1-10%, 0.1-5%, 0.1-3%, about 0.5%, about 1%, about 2%, or about 3% of belantamab is oxidized at amino acid M432. In one embodiment, belantamab comprising ≤86%, oxidation at heavy chain M432 retains ≥70%, FcγRIIIa binding. In another embodiment, belantamab comprising ≤60%, oxidation at heavy chain M432 retains ≥70%, FcRn binding.

In one example, oxidation can be determined using tryptic peptide mapping tandem mass spectrometry (peptide mapping LC-MS/MS). In one example, a sample comprising a composition described herein may be denatured e.g., in 6M guanidine HCl to a concentration of e.g., 4.2 μg/μL. The disulfide bonds may then be reduced e.g., with 50 mM DTT for 20 minutes at room temperature. lodoacetate, e.g., can then be added at e.g., 100 mM and reacted with the free cysteine residues e.g., for 30 minutes at room temperature, protected from light. The sample can then be buffer exchanged e.g., using a BioRad spin columns (part no. 7326221), before digestion e.g., with 0.5% trypsin at for 15 minutes at 37° C. The resulting peptides can then be loaded onto a reversed phase ultra-performance liquid chromatography (UPLC) column and can be eluted with a water and acetonitrile gradient in e.g., 0.1% trifluoroacetic acid using a UPLC. The peptides can then be detected with a UV detector and a mass spectrometer, (e.g., Thermo Scientific LTQ Orbitrap XL). The extracted ion chromatograms of the unmodified and modified peptides are used to calculate the levels of oxidation by dividing the area under the curve of the modified peptide by the total areas under the curve for both modified and unmodified peptides.

In one aspect, a composition comprises an antibody comprising a deamidation post-translational modification (“deamidation” or “deamidated”) or a “deamidated variant”. In one embodiment, the antibody comprises deamidation of an asparagine residue in a CDR of the heavy chain sequence and/or a CDR of the light chain sequence. In a further embodiment, the antibody comprises deamidation of an asparagine residue in a CDR of the heavy chain sequence. In one embodiment, the antibody comprises deamidation of an asparagine residue in the Fc region of the heavy chain sequence and/or the Fc region of the light chain sequence. The deamidated variant may be present in one or both chains of the heavy chain or light chain. It will be understood that these deamidated variant embodiments may be combined with the antibody features described herein. In some embodiments, the deamidated variant comprises one or a combination of deamidation at: N388 and/or N393 of the Fc region of the heavy chain sequence.

In one embodiment, the deamidated variant comprises a deamidated residue selected from: an aspartic acid residue, a succinimide-aspartic acid residue, or an iso-aspartic acid residue.

In one embodiment, the composition comprises an antibody that is at least about 90% identical to the heavy chain amino acid sequence of SEQ ID NO:9 and/or the light chain sequence of SEQ ID NO:10, and comprises deamidation in the heavy chain sequence, e.g., deamidation at amino acid N388 and/or N393.

In one embodiment, the composition comprises a deamidated variant of an anti-BCMA antibody, wherein the deamidated variant comprises a heavy chain amino acid sequence comprising a CDRH1 of SEQ ID NO: 1, a CDRH2 of SEQ ID NO: 2, and a CDRH3 of SEQ ID NO: 3, and a light chain amino acid sequence comprising a CDRL1 of SEQ ID NO: 4, a CDRL2 of SEQ ID NO: 5, and a CDRL3 of SEQ ID NO: 6; wherein the composition comprises up to 100% deamidated variant.

In another embodiment, the composition comprises a deamidated variant of an anti-BCMA antibody, wherein the oxidized variant comprises a heavy chain amino acid sequence comprising a CDRH1 of SEQ ID NO: 1, a CDRH2 of SEQ ID NO: 2, and a CDRH3 of SEQ ID NO: 3, and a light chain amino acid sequence comprising a CDRL1 of SEQ ID NO: 4, a CDRL2 of SEQ ID NO: 5, and a CDRL3 of SEQ ID NO: 6; wherein the composition comprises up to 100% N388 and/or N393 deamidated variant.

In one embodiment, the composition comprises a deamidated variant of belantamab, wherein the deamidated variant comprises a heavy chain sequence of SEQ ID NO: 9 and a light chain sequence of SEQ ID NO: 10; wherein the composition comprises up to 100% deamidated variant. In another embodiment, the composition comprises a deamidated variant comprising a heavy chain sequence of SEQ ID NO:12, SEQ ID NO:13, or SEQ ID NO:14, and a light chain of SEQ ID NO:10.

In another embodiment, a composition comprises an antibody comprising a CDRH1 with the amino acid sequence set forth in SEQ ID NO:1, a CDRH2 with the amino acid sequence set forth in SEQ ID NO:2, a CDRH3 with the amino acid sequence set forth in SEQ ID NO:3, a CDRL1 with the amino acid sequence set forth in SEQ ID NO:4, a CDRL2 with the amino acid sequence set forth in SEQ ID NO:5, a CDRL3 with the amino acid sequence set forth in SEQ ID NO:6, and comprises deamidation in the heavy chain sequence, e.g., deamidation at amino acid N388 and/or N393.

In another embodiment, an anti-BCMA antibody comprises belantamab and comprises deamidation in the heavy chain sequence, e.g., deamidation at amino acid N388 and/or N393.

In one embodiment, a composition comprises a mixture of antibodies at least about 90% identical to the heavy chain amino acid sequence of SEQ ID NO:9 and/or the light chain sequence of SEQ ID NO:10, wherein about ≤100%, ≤75%, ≤60%, ≤50%, ≤40%, 30%, ≤25%, ≤20%, ≤15%, ≤10%, ≤5%, ≤2%, 0.1-100%, 0.1-75%, 0.1-50%, 0.1-40%, 0.1-30%, 0.1-20%, or 0.1-10%, 0.1-5%, 0.1-3%, about 0.5%, about 1%, about 2%, about 5% or about 10% of the antibody in the mixture is deamidated at amino acid N388.

In one embodiment, a composition comprises a mixture of antibodies comprising a CDRH1 with the amino acid sequence set forth in SEQ ID NO:1, a CDRH2 with the amino acid sequence set forth in SEQ ID NO:2, a CDRH3 with the amino acid sequence set forth in SEQ ID NO:3, a CDRL1 with the amino acid sequence set forth in SEQ ID NO:4, a CDRL2 with the amino acid sequence set forth in SEQ ID NO:5, a CDRL3 with the amino acid sequence set forth in SEQ ID NO:6, wherein about ≤100%, ≤75%, ≤60%, ≤50%, ≤40%, 30%, ≤25%, ≤20%, ≤15%, ≤10%, ≤5%, ≤2%, 0.1-100%, 0.1-75%, 0.1-50%, 0.1-40%, 0.1-30%, 0.1-20%, or 0.1-10%, 0.1-5%, 0.1-3%, about 0.5%, about 1%, about 2%, about 5% or about 10% of the antibody in the mixture is deamidated at amino acid N388.

In another embodiment, a composition comprises belantamab, wherein about ≤100%, ≤75%, ≤60%, ≤50%, ≤40%, ≤30%, ≤25%, ≤20%, ≤15%, ≤10%, ≤5%, ≤2%, 0.1-100%, 0.1-75%, 0.1-50%, 0.1-40%, 0.1-30%, 0.1-20%, or 0.1-10%, 0.1-5%, 0.1-3%, about 0.5%, about 1%, about 2%, about 5% or about 10% of belantamab is deamidated at amino acid N388.

In one embodiment, a composition comprises a mixture of antibodies at least about 90% identical to the heavy chain amino acid sequence of SEQ ID NO:9 and/or the light chain sequence of SEQ ID NO:10, wherein about 100%, ≤85%, ≤70%, ≤60%, ≤50%, 40%, ≤30%, ≤20%, ≤15%, ≤10%, ≤5%, ≤2%, 0.1-100%, 0.1-75%, 0.1-50%, 0.1-40%, 0.1-30%, 0.1-20%, or 0.1-10%, 0.1-5%, 0.1-3%, about 0.5%, about 1%, about 2%, about 5% or about 10% of the antibody in the mixture is deamidated at amino acid N393.

In one embodiment, a composition comprises a mixture of antibodies comprising a CDRH1 with the amino acid sequence set forth in SEQ ID NO:1, a CDRH2 with the amino acid sequence set forth in SEQ ID NO:2, a CDRH3 with the amino acid sequence set forth in SEQ ID NO:3, a CDRL1 with the amino acid sequence set forth in SEQ ID NO:4, a CDRL2 with the amino acid sequence set forth in SEQ ID NO:5, a CDRL3 with the amino acid sequence set forth in SEQ ID NO:6, wherein about ≤100%, ≤85%, ≤70%, ≤60%, ≤50%, 40%, ≤30%, ≤20%, ≤15%, ≤0%, ≤5%, ≤2%, 0.1-100%, 0.1-75%, 0.1-50%, 0.1-40%, 0.1-30%, 0.1-20%, or 0.1-10%, 0.1-5%, 0.1-3%, about 0.5%, about 1%, about 2%, about 5% or about 10% of the antibody in the mixture is deamidated at amino acid N393.

In another embodiment, a composition comprises belantamab, wherein about ≤100%, ≤85%, ≤70%, ≤60%, ≤50%, ≤40%, ≤30%, ≤20%, ≤15%, ≤10%, ≤5%, ≤2%, 0.1-100%, 0.1-75%, 0.1-50%, 0.1-40%, 0.1-30%, 0.1-20%, or 0.1-10%, 0.1-5%, 0.1-3%, about 0.5%, about 1%, about 2%, about 5% or about 10% of belantamab is deamidated at amino acid N393.

In one example, deamidation can be determined using tryptic peptide mapping tandem mass spectrometry (peptide mapping LC-MS/MS). In one example, a sample comprising a composition described herein may be denatured e.g., in 6M guanidine HCl to a concentration of e.g., 4.2 μg/μL. The disulfide bonds may then be reduced e.g., with 50 mM DTT for 20 minutes at room temperature. lodoacetate, e.g., can then be added at e.g., 100 mM and reacted with the free cysteine residues e.g., for 30 minutes at room temperature, protected from light. The sample can then be buffer exchanged e.g., using a BioRad spin columns (part no. 7326221), before digestion e.g., with 0.5% trypsin at for 15 minutes at 37° C. The resulting peptides can then be loaded onto a reversed phase ultra-performance liquid chromatography (UPLC) column and can be eluted with a water and acetonitrile gradient in e.g., 0.1% trifluoroacetic acid using a UPLC. The peptides can then be detected with a UV detector and a mass spectrometer, (e.g., Thermo Scientific LTQ Orbitrap XL). The extracted ion chromatograms of the unmodified and modified peptides are used to calculate the levels of deamidation by dividing the area under the curve of the modified peptide by the total areas under the curve for both modified and unmodified peptides.

In one embodiment, a post-translational modification is an antibody sequence variant. Exemplary post-translational modification antibody sequence variants comprise an asparagine (N) to aspartic acid (D) switch, an N-terminal pyro-glutamate, and/or a C-terminal lysine cleavage.

In one example, antibody variants, e.g., N103D at CDRH3, can be determined using tryptic peptide mapping tandem mass spectrometry (peptide mapping LC-MS/MS). In one example, a sample comprising a composition described herein may be denatured e.g., in 6M guanidine HCl to a concentration of e.g., 4.2 μg/μL. The disulfide bonds may then be reduced e.g., with 50 mM DTT for 20 minutes at room temperature. lodoacetate, e.g., can then be added at e.g., 100 mM and reacted with the free cysteine residues e.g., for 30 minutes at room temperature, protected from light. The sample can then be buffer exchanged e.g., using a BioRad spin columns (part no. 7326221), before digestion e.g., with 0.5% trypsin at for 15 minutes at 37° C. The resulting peptides can then be loaded onto a reversed phase ultra-performance liquid chromatography (UPLC) column and can be eluted with a water and acetonitrile gradient in e.g., 0.1% trifluoroacetic acid using a UPLC.

The peptides can then be detected with a UV detector and a mass spectrometer, (e.g., Thermo Scientific LTQ Orbitrap XL). The extracted ion chromatograms of the unmodified and modified peptides are used to calculate the levels of antibody variant, e.g., N103D at CDRH3, by dividing the area under the curve of the modified peptide by the total areas under the curve for both modified and unmodified peptides.

In one aspect, a composition comprises an antibody comprising an N-terminal pyroglutamic acid (“pyroglutamic acid”) post-transitional modification in the heavy chain amino acid sequence. In one embodiment, the composition comprises an antibody that is at least about 90% identical to the heavy chain amino acid sequence of SEQ ID NO:9 and/or the light chain sequence of SEQ ID NO:10, and comprises pyroglutamic acid at the N-terminus of the heavy chain.

In another embodiment, a composition comprises an antibody comprising a CDRH1 with the amino acid sequence set forth in SEQ ID NO:1, a CDRH2 with the amino acid sequence set forth in SEQ ID NO:2, a CDRH3 with the amino acid sequence set forth in SEQ ID NO:3, a CDRL1 with the amino acid sequence set forth in SEQ ID NO:4, a CDRL2 with the amino acid sequence set forth in SEQ ID NO:5, a CDRL3 with the amino acid sequence set forth in SEQ ID NO:6, and comprises pyroglutamic acid at the N-terminus of the heavy chain.

In another embodiment, an anti-BCMA antibody comprises belantamab and comprises pyroglutamic acid at the N-terminus of the heavy chain.

In one embodiment, a composition comprises a mixture of antibodies at least about 90% identical to the heavy chain amino acid sequence of SEQ ID NO:9 and/or the light chain sequence of SEQ ID NO:10, wherein about ≥25%, ≥50%, ≥75%, ≥80%, ≥85%, 90%, ≥95%, 100% or less, 95% or less, 90% or less, 85% or less, 80% or less, 75% or less, or 50% or less of the antibody in the mixture comprises N-terminal pyroglutamic acid in the heavy chain amino acid sequence.

In one embodiment, a composition comprises a mixture of antibodies comprising a CDRH1 with the amino acid sequence set forth in SEQ ID NO:1, a CDRH2 with the amino acid sequence set forth in SEQ ID NO:2, a CDRH3 with the amino acid sequence set forth in SEQ ID NO:3, a CDRL1 with the amino acid sequence set forth in SEQ ID NO:4, a CDRL2 with the amino acid sequence set forth in SEQ ID NO:5, a CDRL3 with the amino acid sequence set forth in SEQ ID NO:6, wherein about ≥25%, ≥50%, ≥75%, ≥80%, ≥85%, 90%, ≥95%, 100% or less, 95% or less, 90% or less, 85% or less, 80% or less, 75% or less, or 50% or less of the antibody in the mixture comprises N-terminal pyroglutamic acid in the heavy chain amino acid sequence.

In one embodiment, a composition comprises belantamab, wherein about ≥25%, ≥50%, ≥75%, ≥80%, ≥85%, ≥90%, ≥95%, 100% or less, 95% or less, 90% or less, 85% or less, 80% or less, 75% or less, or 50% or less of belantamab comprises N-terminal pyroglutamic acid in the heavy chain amino acid sequence.

In one example, N-terminal pyroglutamic acid can be determined using tryptic peptide mapping tandem mass spectrometry (peptide mapping LC-MS/MS). In one example, a sample comprising a composition described herein may be denatured e.g., in 6M guanidine HCl to a concentration of e.g., 4.2 μg/μL. The disulfide bonds may then be reduced e.g., with 50 mM DTT for 20 minutes at room temperature. lodoacetate, e.g., can then be added at e.g., 100 mM and reacted with the free cysteine residues e.g., for 30 minutes at room temperature, protected from light. The sample can then be buffer exchanged e.g., using a BioRad spin columns (part no. 7326221), before digestion e.g., with 0.5% trypsin at for 15 minutes at 37° C. The resulting peptides can then be loaded onto a reversed phase ultra-performance liquid chromatography (UPLC) column and can be eluted with a water and acetonitrile gradient in e.g., 0.1% trifluoroacetic acid using a UPLC.

The peptides can then be detected with a UV detector and a mass spectrometer, (e.g., Thermo Scientific LTQ Orbitrap XL). The extracted ion chromatograms of the unmodified and modified peptides are used to calculate the levels of pyroglutamic acid by dividing the area under the curve of the modified peptide by the total areas under the curve for both modified and unmodified peptides.

In one aspect, a composition comprises an antibody comprising a C-terminal lysine cleavage post-translational modification in the heavy chain amino acid sequence. In one embodiment, the composition comprises an antibody that is at least about 90% identical to the heavy chain amino acid sequence of SEQ ID NO:9 and/or the light chain sequence of SEQ ID NO:10, and comprises a C-terminal lysine cleavage of the heavy chain.

In another embodiment, a composition comprises an antibody comprising a CDRH1 with the amino acid sequence set forth in SEQ ID NO:1, a CDRH2 with the amino acid sequence set forth in SEQ ID NO:2, a CDRH3 with the amino acid sequence set forth in SEQ ID NO:3, a CDRL1 with the amino acid sequence set forth in SEQ ID NO:4, a CDRL2 with the amino acid sequence set forth in SEQ ID NO:5, a CDRL3 with the amino acid sequence set forth in SEQ ID NO:6, and comprises and comprises a C-terminal lysine cleavage of the heavy chain.

In another embodiment, an anti-BCMA antibody comprises belantamab and comprises a C-terminal lysine cleavage of the heavy chain.

In one embodiment, a composition comprises a mixture of antibodies at least about 90% identical to the heavy chain amino acid sequence of SEQ ID NO:9 and/or the light chain sequence of SEQ ID NO:10, wherein about ≥25%, ≥50%, ≥75%, ≥80%, ≥85%, ≥90%, ≥95%, 100% or less, 95% or less, 90% or less, 85% or less, 80% or less, 75% or less, or 50% or less of the antibody in the mixture comprises a C-terminal lysine cleavage.

In one embodiment, a composition comprises a mixture of antibodies comprising a CDRH1 with the amino acid sequence set forth in SEQ ID NO:1, a CDRH2 with the amino acid sequence set forth in SEQ ID NO:2, a CDRH3 with the amino acid sequence set forth in SEQ ID NO:3, a CDRL1 with the amino acid sequence set forth in SEQ ID NO:4, a CDRL2 with the amino acid sequence set forth in SEQ ID NO:5, a CDRL3 with the amino acid sequence set forth in SEQ ID NO:6, wherein about ≥25%, ≥50%, ≥75%, ≥80%, ≥85%, ≥90%, ≥95%, 100% or less, 95% or less, 90% or less, 85% or less, 80% or less, 75% or less, or 50% or less of the antibody in the mixture comprises a C-terminal lysine cleavage of the heavy chain.

In one embodiment, a composition comprises belantamab, wherein about ≥25%, ≥50%, ≥75%, ≥80%, ≥85%, ≥90%, ≥95%, 100% or less, 95% or less, 90% or less, 85% or less, 80% or less, 75% or less, or 50% or less of belantamab comprises a C-terminal lysine cleavage of the heavy chain.

In one example, C-terminal lysine cleavage can be determined using tryptic peptide mapping tandem mass spectrometry (peptide mapping LC-MS/MS). In one example, a sample comprising a composition described herein may be denatured e.g., in 6M guanidine HCl to a concentration of e.g., 4.2 μg/μL. The disulfide bonds may then be reduced e.g., with 50 mM DTT for 20 minutes at room temperature. lodoacetate, e.g., can then be added at e.g., 100 mM and reacted with the free cysteine residues e.g., for 30 minutes at room temperature, protected from light. The sample can then be buffer exchanged e.g., using a BioRad spin columns (part no. 7326221), before digestion e.g., with 0.5% trypsin at for 15 minutes at 37° C. The resulting peptides can then be loaded onto a reversed phase ultra-performance liquid chromatography (UPLC) column and can be eluted with a water and acetonitrile gradient in e.g., 0.1% trifluoroacetic acid using a UPLC. The peptides can then be detected with a UV detector and a mass spectrometer, (e.g., Thermo Scientific LTQ Orbitrap XL). The extracted ion chromatograms of the unmodified and modified peptides are used to calculate the levels of C-terminal lysine cleavage by dividing the area under the curve of the modified peptide by the total areas under the curve for both modified and unmodified peptides.

In one aspect, a composition comprises an antibody comprising glycosylation post-translational modification (“glycosylation modification”) or glycosylation variant. Exemplary glycosylation modifications include changes to the expression of G0, G1, G0-GlcNac, G2, and sialylation on the antibody. In one embodiment, a composition comprises an antibody that is at least about 90% identical to the heavy chain amino acid sequence of SEQ ID NO:9 and/or the light chain sequence of SEQ ID NO:10, and comprises a glycosylation modification.

In another embodiment, a composition comprises an antibody comprising a CDRH1 with the amino acid sequence set forth in SEQ ID NO:1, a CDRH2 with the amino acid sequence set forth in SEQ ID NO:2, a CDRH3 with the amino acid sequence set forth in SEQ ID NO:3, a CDRL1 with the amino acid sequence set forth in SEQ ID NO:4, a CDRL2 with the amino acid sequence set forth in SEQ ID NO:5, a CDRL3 with the amino acid sequence set forth in SEQ ID NO:6, and comprises a glycosylation variant.

In another embodiment, an anti-BCMA antibody comprises belantamab and comprises a glycosylation variant.

In one embodiment, a composition comprises a mixture of antibodies at least about 90% identical to the heavy chain amino acid sequence of SEQ ID NO:9 and/or the light chain sequence of SEQ ID NO:10, wherein the composition comprises G0 at a level of about ≥25%, ≥30%, ≥35%, ≥40%, ≥45%, ≥50%, ≥55%, ≥60%, 0-100%, 1-100%, 30-100%, 40-90%, 50-80%, or 55-80%; G1 at a level of about ≥2.5%, ≥5%, ≥10%, ≥15%, ≥20%, ≥25%, ≥30%, ≥50%, 0-100%, 1-100%, 0-50%, 1-50%, 1-40%, 1-35%, or 8-31%; G0-GlcNac at a level of about ≤5%, ≤7.5%, ≤10%, ≤15%, ≤20%, ≤25%, ≤30%, ≤40%, ≤50%, ≤75%, 0-100%, 0.5-100%, 0-50%, 0.5-50%, 0.5-25%, 0.5-10%, 0.5-7.5%, or 0.9-5.3%; G2 at a level of 0-100%, 1-100% or 39-92%; and/or G0-2GlcNac at a level of 0-100%, 1-100%, or 38-88%.

In one embodiment, a composition comprises a mixture of antibodies comprising a CDRH1 with the amino acid sequence set forth in SEQ ID NO:1, a CDRH2 with the amino acid sequence set forth in SEQ ID NO:2, a CDRH3 with the amino acid sequence set forth in SEQ ID NO:3, a CDRL1 with the amino acid sequence set forth in SEQ ID NO:4, a CDRL2 with the amino acid sequence set forth in SEQ ID NO:5, a CDRL3 with the amino acid sequence set forth in SEQ ID NO:6, wherein the composition comprises G0 at a level of about ≥25%, ≥30%, ≥35%, ≥40%, ≥45%, ≥50%, ≥55%, ≥60%, 0-100%, 1-100%, 30-100%, 40-90%, 50-80% or 55-80%; G1 at a level of about ≥2.5%, ≥5%, ≥10%, ≥15%, ≥20%, ≥25%, ≥30%, ≥50%, 0-100%, 1-100%, 0-50%, 1-50%, 1-40%, 1-35%, or 8-31%; G0-GlcNac at a level of about ≤5%, ≤7.5%, ≤10%, ≤15%, ≤20%, ≤25%, ≤30%, ≤40%, ≤50%, ≤75%, 0-100%, 0.5-100%, 0-50%, 0.5-50%, 0.5-25%, 0.5-10%, 0.5-7.5% or 0.9-5.3%; G2 at a level of 0-100%, 1-100% or 39-92%; and/or G0-2GlcNac at a level of 0-100%, 1-100%, or 38-88%.

In one embodiment, a composition comprises belantamab, wherein the composition comprises G0 at a level of about ≥25%, ≥30%, ≥35%, ≥40%, ≥45%, ≥50%, ≥55%, ≥60%, 0-100%, 1-100%, 30-100%, 40-90%, 50-80% or 55-80%; G1 at a level of about ≥2.5%, ≥5%, ≥10%, ≥15%, ≤20%, ≤25%, ≤30%, ≤50%, 0-100%, 1-100%, 0-50%, 1-50%, 1-40%, 1-35%, or 8-31%; G0-GlcNac at a level of about ≤5%, ≤7.5%, ≤10%, ≤15%, ≤20%, ≤25%, ≤30%, ≤40%, ≤50%, ≤75%, 0-100%, 0.5-100%, 0-50%, 0.5-50%, 0.5-25%, 0.5-10%, 0.5-7.5% or 0.9-5.3%; G2 at a level of 0-100%, 1-100% or 39-92%; and/or G0-2GlcNac at a level of 0-100%, 1-100%, or 38-88%.

In one embodiment, a composition comprises a mixture of antibodies wherein 100% are afucosylated. In another embodiment, a composition comprises a mixture of antibodies wherein 0% are fucosylated.

In one example, glycosylation modifications and the resulting profile can be determined using Ultra Performance Liquid Chromatography (UPLC) with Hydrophilic Interaction Liquid Chromatography (HILIC) separation and fluorescence detection. In one example, a composition described herein, e.g., comprising belantamab, can be diluted to a concentration of 10 μg/μL with water, and the glycans may then be released from the composition e.g., comprising belantamab, by an enzymatic digestion with PNGaseF using a PNGase F kit from New England BioLabs, (Cat #P0705L). The glycans can be released by PNGase F and labelled with anthranilamide (Sigma-Aldrich, Cat #A89804). The labelled glycans can then purified to remove excess labelling solution using a HILIC column step; the glycans can be loaded and washed with water and eluting with acetonitrile. The labelled glycans can then be separated using a Waters Glycan BEH Amide column (cat no. 186004742) on a Waters Acquity UPLC with an ammonium formate/formic acid and acetonitrile gradient. The glycans can then be detected, e.g., by using fluorescence detection with excitation at 365 nm and emission at 438 nm. Quantitation of the glycans can achieved, e.g., by dividing the area under the curve of a glycan by the total area under the curve for all detected glycans.

In one aspect, the composition comprises antibodies that are aggregated antibodies (High molecular weight (HMW) species) also referred to herein as an “aggregated variant”. The aggregated antibodies may comprise dimers or higher order structures formed of antibody monomers and subunits thereof. Aggregated variants can be, for example, covalent or non-covalent, reducible or non-reducible, and visible or subvisible aggregates of an antibody disclosed herein. Aggregated or fragmented variants can be characterized and distinguished from an antibody based on their size. For example, the size distribution of an antibody composition can be detected using size exclusion chromatography (SEC), such as SE-HPLC.

In one aspect, the composition comprises an aggregated variant of the antibody, wherein the aggregated variant comprises a heavy chain sequence comprising a CDRH1 of SEQ ID NO: 1, a CDRH2 of SEQ ID NO: 2, and a CDRH3 of SEQ ID NO: 3, and a light chain sequence comprising a CDRL1 of SEQ ID NO: 4, a CDRL2 of SEQ ID NO: 5, and a CDRL3 of SEQ ID NO: 6; wherein the composition comprises ≤10% aggregated variant.

The antibody composition may comprise ≤10% aggregated variant, such as ≤7.5%, ≤5%, ≤3%, ≤2%, or ≤1% aggregated variant. In another embodiment, the composition may comprise 1-10%, 1-5%, 1-4%, 1-3%, or 1-2% aggregated variant. Alternatively, the composition comprises more than 1% and less than 10% aggregated variant. Alternatively, the composition may comprise about 7.5%, about 5%, about 4%, about 3%, about 2%, or about 1% aggregated variant.

Fragmented variants (“fragment variant”) are variants which comprise a portion of a full length antibody. For example, such fragments include Fab, Fab′, F(ab′)2, and Fv fragments, diabodies, linear antibodies, single-chain antibody molecules and immunoglobulin single variable domains.

In one aspect, the composition comprises a fragment variant of the antibody, wherein the fragment variant comprises a heavy chain sequence comprising a CDRH1 of SEQ ID NO: 1, a CDRH2 of SEQ ID NO: 2, and a CDRH3 of SEQ ID NO: 3, and a light chain sequence comprising a CDRL1 of SEQ ID NO: 4, a CDRL2 of SEQ ID NO: 5, and a CDRL3 of SEQ ID NO: 6; wherein the composition comprises ≤0% fragment variant.

The antibody composition may comprise ≤10%, fragmented antibodies, such as ≤5%, ≤3%, ≤2%, or ≥1%, fragmented antibodies. In another embodiment, the composition may comprise 0.5-10%, 0.5-5%, 0.5-4%, 0.5-3%, 0.5-2%, 0.5-1.5%, or 0.5-1% fragmented antibodies. Alternatively, the composition may comprise about 5%, about 4%, about 3%, about 2%, about 1%, or about 0.5% fragmented antibodies.

A composition may comprise any one or a combination of the acidic, basic, isomerization, oxidation, deamidation, N-terminal pyroglutamic acid, C-terminal lysine cleavage variants, and/or any percentage of the glycosylation modification variants, and/or aggregated and/or fragmented variants as described herein.

In one embodiment a composition has ≥70%, BCMA specific antigen binding, ≥70%, FcγRIIIa binding, and/or ≥70% FcRn binding.

In another embodiment a composition has about ≥75%, ≥80%, ≥85%, ≥90%, or ≥95% BCMA specific antigen binding. In another embodiment a composition has about ≥75%, ≥80%, ≥85%, ≥90%, ≥95% FcγRIIIa binding. In another embodiment a composition has about ≥75%, ≥80%, ≥85%, ≥90%, ≥95% FcRn binding.

In another embodiment a composition has a specific antigen binding in the range of about 70% to 130%, FcγRIIIa binding in the range of from about 70% to 130%, and/or FcRn binding in the range of from about 70% to 130%.

In some embodiments a composition has a specific antigen binding in the range of about 75% to about 125%, about 80% to about 120%, about 90% to about 110%, about 70%, about 80%, about 90%, or 100%, about 110%, about 120%, or about 130%. In some embodiments a composition has a FcγRIIIa binding in the range of about 75% to about 125%, about 80% to about 120%, about 90% to about 110%, about 90%, about 95%, about 100%, about 105%, or about 110%. In some embodiments a composition has a FcRn binding in the range of about 75% to about 125%, about 80% to about 120%, about 90% to about 110%, about 90%, about 95%, about 100%, about 105%, about 110%.

In another embodiment the composition comprising a variant has at least 70%, at least 75%, at least 80%, at least 85%, at least 90%, or at least 95% of the activity of belantamab which has 100% activity. In one aspect, the composition comprises a variant comprising a heavy chain amino acid sequence comprising a CDRH1 of SEQ ID NO: 1, a CDRH2 of SEQ ID NO: 2, and a CDRH3 of SEQ ID NO: 3, and a light chain amino acid sequence comprising a CDRL1 of SEQ ID NO: 4, a CDRL2 of SEQ ID NO: 5, and a CDRL3 of SEQ ID NO: 6; wherein the composition has at least 70% of the potency of a composition comprising a heavy chain sequence of SEQ ID NO: 9, 11, 12, 13 or 14 and a light chain sequence of SEQ ID NO: 10 and any one or a combination of: (i) up to 23% isomerization at D103, and/or (ii) up to 37% oxidation at M34.

In another aspect, the composition comprises a variant comprising a heavy chain amino acid sequence comprising a CDRH1 of SEQ ID NO: 1, a CDRH2 of SEQ ID NO: 2, and a CDRH3 of SEQ ID NO: 3, and a light chain amino acid sequence comprising a CDRL1 of SEQ ID NO: 4, a CDRL2 of SEQ ID NO: 5, and a CDRL3 of SEQ ID NO: 6; wherein the composition has at least 70% of the potency of a composition comprising a heavy chain sequence of SEQ ID NO: 9, 11, 12, 13 or 14 and a light chain sequence of SEQ ID NO: 10 and any one or a combination of: (i) up to 23% isomerization at D103, (ii) up to 37% oxidation at M34, (iii) up to 64% oxidation at M256, (iv) up to 61% oxidation at M432, (v) up to 100% deamidation at N388, and/or (vi) up to 100% deamidation at N393. In yet another aspect, the composition comprises a variant comprising a heavy chain amino acid sequence comprising a CDRH1 of SEQ ID NO: 1, a CDRH2 of SEQ ID NO: 2, and a CDRH3 of SEQ ID NO: 3, and a light chain amino acid sequence comprising a CDRL1 of SEQ ID NO: 4, a CDRL2 of SEQ ID NO: 5, and a CDRL3 of SEQ ID NO: 6; wherein the composition has at least 70% of the potency of a composition comprising a heavy chain sequence of SEQ ID NO: 9, 11, 12, 13 or 14 and a light chain sequence of SEQ ID NO: 10 and any one or a combination of: (i) up to 23% isomerization at D103, (ii) up to 37% oxidation at M34, (iii) up to 64% oxidation at M256, (iv) up to 61% oxidation at M432, (v) up to 100% deamidation at N388, (vi) up to 100% deamidation at N393, (vii) up to 100% HC C-terminal lysine cleavage, and/or (viii) up to 100% HC N-terminal pyroglutamic acid.

In one example, the binding of BCMA and FcγRIIIa by belantamab mafodotin is measured using surface plasmon resonance (SPR). Belantamab mafodotin may be diluted to 10 μg/mL with PBST, injected and captured by protein A immobilized on a CM5 sensor chip. BCMA may then be injected and bound to the captured belantamab mafodotin. Next, FcγRIIIa may be injected and bound to the captured belantamab mafodotin. The functional concentrations of belantamab mafodotin binding to BCMA and FcγRIIIa can be calculated from a reference standard curve and reported as the BCMA or FcγRIIIa binding concentration, respectively. The total belantamab mafodotin concentration of the sample is pre-determined by absorbance at 280 nm. The specific binding activity (%) can be calculated by dividing the BCMA or FcγRIIIa binding concentration by the absorbance at 280 nm concentration,

The binding of Neonatal Fc Receptor (FcRn) to an anti-BCMA antigen binding protein, e.g., belantamab, can measured using surface plasmon resonance (SPR). Belantamab can be captured by FcRn, which is immobilized on a nitrilotriacetic acid (NTA) sensor chip. The FcRn binding concentration of the sample can be determined by interpolation of the binding response on a calibration curve. Specific binding activity (%) is calculated by dividing the FcRn binding concentration by the total protein concentration.

Among other methods known to those skilled in the art, the binding of BCMA and FcγRIIIa by an anti-BCMA antigen binding protein, e.g., belantamab mafodotin, can measured using surface plasmon resonance (SPR). In one example, belantamab mafodotin is injected and captured by protein A immobilized on a CM5 sensor chip. BCMA is then injected and bound to the captured belantamab mafodotin. Next, FcγRIIIa is injected and bound to the captured belantamab mafodotin. The functional concentrations of belantamab mafodotin binding to BCMA and FcγRIIIa can be calculated from a reference standard curve and reported as the BCMA or FcγRIIIa binding concentration, respectively. The total belantamab mafodotin concentration of the sample can be pre-determined by absorbance at 280 nm. The specific binding activity (%) can be calculated by dividing the BCMA or FcγRIIIa binding concentration by the absorbance at, e.g., 280 nm concentration.

In certain embodiments, average DAR or percent DL impacts binding to FcRn. In another embodiment, average DAR or percent DL does not impact binding to FcRn. In yet another embodiment, a composition comprises belantamab mafodotin, and the average DAR or percent DL impacts binding to FcRn. In yet another embodiment, a composition comprises belantamab mafodotin, and average DAR or percent DL does not impact binding to FcRn. In one embodiment, average DAR or percent DL may weaken binding to FcRn.

The binding of Neonatal Fc Receptor (FcRn) to an anti-BCMA antigen binding protein, e.g., belantamab mafodotin, can measured using surface plasmon resonance (SPR). Belantamab mafodotin can be captured by FcRn, which is immobilized on a nitrilotriacetic acid (NTA) sensor chip. The FcRn binding concentration of the sample can be determined by interpolation of the binding response on a calibration curve. Specific binding activity (%) is calculated by dividing the FcRn binding concentration by the total protein concentration.

When an anti-BCMA antigen binding protein comprises belantamab mafodotin, the SPR methods described herein for specific antigen binding, FcγRIIIa and FcRn binding may use a reference standard of belantamab or belantamab mafodotin. The belantamab or belantamab mafodotin reference standard can be used in assays to obtain system suitability and sample comparability data, to ensure methods are performing appropriately. The reference standard can allow the establishment of a calibration curve and concentrations of the samples are interpolated from the curve. For example, a reference standard may be a composition comprising a heavy chain amino acid sequence of SEQ ID NO:9 and a light chain amino acid sequence of SEQ ID NO:10

The antibody composition comprising the antibody and antibody variants described above retain specific antigen binding and/or FcRn binding and/or FcγRIIIa binding and/or potency. For example, the antibody composition comprising the antibody and antibody variants and post-translational modification variants described above has >0.70 BCMA specific antigen binding; and/or >70% FcRn binding and/or 70% FcγRIIIa binding and/or >70% potency. Thus these levels (%) of variants can be tolerated in the antibody composition without significantly impacting function (i.e. without resulting in reduced activity). In one embodiment, “reduced function” or “reduced activity” means that binding to BCMA, or binding to FcRn, or binding to FcγRIIIa, or potency is reduced as a percentage compared to a reference standard, and is significant over assay variability. For example, reduced function or activity or potency can be described as a reduction of ≥5%, ≥10%, ≥15%, ≥20%, ≥25%, ≥30%, ≥35%, ≥40%, ≥45%, or ≥50%.

In another embodiment, the reference sample standard is a composition comprising a heavy chain amino acid sequence of SEQ ID NO:9 and a light chain amino acid sequence of SEQ ID NO:10 wherein the composition comprises 80% or more heavy chain C-terminal lysine cleavage and 100% or less heavy chain N-terminal pyroglutamic acid. In a further embodiment, the reference sample standard is a composition comprising a heavy chain amino acid sequence of SEQ ID NO:9 and a light chain amino acid sequence of SEQ ID NO:10 wherein the composition comprises 80% or more heavy chain C-terminal lysine cleavage and 100% or less heavy chain N-terminal pyroglutamic acid, and 7% or less isomerization at amino acid D103 at CDRH3. In a further embodiment, the reference sample standard is a composition comprising a heavy chain amino acid sequence of SEQ ID NO:9 and a light chain amino acid sequence of SEQ ID NO:10 wherein the composition comprises 80% or more heavy chain C-terminal lysine cleavage and 100% or less heavy chain N-terminal pyroglutamic acid, 7% or less isomerization at amino acid D103 at CDRH3, and 5% or less oxidation at amino acids M34, M256 and/or M432. In a further embodiment, the reference sample standard is a composition comprising a heavy chain amino acid sequence of SEQ ID NO:9 and a light chain amino acid sequence of SEQ ID NO:10 wherein the composition comprises 80% or more heavy chain C-terminal lysine cleavage and 100% or less heavy chain N-terminal pyroglutamic acid, 7% or less isomerization at amino acid D103 at CDRH3, 5% or less oxidation at amino acids M34, M256 and/or M432, and 2% or less deamidation at amino acids N388 and/or N393. In a further embodiment, the reference sample standard is a composition comprising a heavy chain amino acid sequence of SEQ ID NO:9 and a light chain amino acid sequence of SEQ ID NO:10 wherein the composition comprises 80% or more heavy chain C-terminal lysine cleavage and 100% heavy chain N-terminal pyroglutamic acid, 7% or less isomerization at amino acid D103 at CDRH3, 5% or less oxidation at M256, 2% or less oxidation at M34 and M432, and 2% or less deamidation at amino acids N388 and N393.

### Antibody-Drug Conjugates (ADCs)

Antibody drug conjugates (ADC) are an emerging class of potent anti-cancer agents, which have recently demonstrated remarkable clinical benefit. ADCs are comprised of a cytotoxic agent chemically bound to an antibody via a linker. Putatively, by a series of events, including antigen binding at the cell surface, endocytosis, trafficking to the lysosome, ADC degradation, release of payload, interruption of cellular processing (e.g., mitosis) and apoptosis, ADCs may destroy cancer cells possessing an over-expression of cell-surface proteins. ADCs combine the antigen-driven targeting properties of monoclonal antibodies with the potent anti-tumor effects of cytotoxic agents. For example, in 2011 ADCETRIS® (an anti-CD30 antibody-MMAE ADC) gained regulatory approval for the treatment of refractory Hodgkin lymphoma and systemic anaplastic lymphoma.

ADCs have been used for the local delivery of cytotoxic agents, i.e., drugs that kill or inhibit the growth or proliferation of cells, in the treatment of cancer (Lambert, J. (2005) Curr. Opinion in Pharmacology 5:543-549; Wu et al. (2005) Nature Biotechnology 23(9):1137-1146; Payne, G. (2003) i 3:207-212; Syrigos and Epenetos (1999) Anticancer Research 19:605-614; Niculescu-Duvaz and Springer (1997) Adv. Drug Deliv. Rev. 26:151-172; U.S. Pat. No. 4,975,278). ADCs allow for the targeted delivery of a drug moiety to a tumor, and intracellular accumulation therein, where systemic administration of unconjugated drugs may result in unacceptable levels of toxicity to normal cells as well as the tumor cells sought to be eliminated (Baldwin et al., Lancet (Mar. 15, 1986) pp. 603-05; Thorpe (1985) “Antibody Carriers Of Cytotoxic Agents In Cancer Therapy: A Review,” in Monoclonal Antibodies '84: Biological And Clinical Applications (A. Pinchera et al., eds) pp. 475-506. Both polyclonal antibodies and monoclonal antibodies have been reported as useful in these strategies (Rowland et al., (1986) Cancer Immunol. Immunother. 21:183-87). Toxins used in antibody-toxin conjugates include bacterial toxins such as diphtheria toxin, plant toxins such as ricin, small molecule toxins such as geldanamycin (Mandler et al (2000) J. Nat. Cancer Inst. 92(19):1573-1581; Mandler et al. (2000) Bioorganic & Med. Chem. Letters 10:1025-1028; Mandler et al (2002) Bioconjugate Chem. 13:786-791), maytansinoids (EP 1391213; Liu et al. (1996) Proc. Natl. Acad. Sci. USA 93:8618-8623), and calicheamicin (Lode et al (1998) Cancer Res. 58:2928; Hinman et al. (1993) Cancer Res. 53:3336-3342).

In one embodiment, an anti-BCMA antigen binding protein is an antibody-drug conjugate (“anti-BCMA ADC”) comprising an antibody or antibody fragment conjugated to one or more cytotoxic agents, such as a chemotherapeutic agent, a drug, a growth inhibitory agent, a toxin (e.g., a protein toxin, an enzymatically active toxin of bacterial, fungal, plant, or animal origin, or fragments thereof), or a radioactive isotope (i.e., a radioconjugate).

In one embodiment, an anti-BCMA ADC has the following general structure:

ABP-((Linker)ₙ-Ctx)ₘ

Wherein:
ABP is an antigen binding protein, antibody, or antibody fragment;
Linker is either absent or any a cleavable or non-cleavable linker;
Ctx is any cytotoxic agent described herein;
n is 0, 1, 2, or 3; and,
m is 1, 2, 3, 4, 5, 6, 7, 8, 9 or 10.

In exemplary embodiments, enzymatically active toxins and fragments thereof that could be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, or the tricothecenes. See, e.g., WO 93/21232 published Oct. 28, 1993. A variety of radionuclides are available for the production of radio-conjugated antibodies, including, e.g., ²¹¹At, ²¹²Bi, ¹³¹I, ¹³¹In, ⁹⁰Y, or ¹⁸⁶Re.

An anti-BCMA antibody or fragments thereof of the present invention may also be conjugated to one or more cytotoxic agents, including, but not limited to, a calicheamicin, maytansinoids, dolastatins, auristatins, a trichothecene, and CC1065, or derivatives of these toxins that have toxin activity. Suitable cytotoxic agents include, for example, an auristatin including dovaline-valine-dolaisoleunine-dolaproinephenylalanine (MMAF) and monomethyl auristatin E (MMAE) as well as ester forms of MMAE, a DNA minor groove binding agent, a DNA minor groove alkylating agent, an enediyne, a lexitropsin, a duocarmycin, a taxane, including paclitaxel and docetaxel, a puromycin, a dolastatin, a maytansinoid, and a vinca alkaloid. Specific cytotoxic agents include topotecan, morpholino-doxorubicin, rhizoxin, cyanomorpholino-doxorubicin, dolastatin-10, echinomycin, combretatstatin, chalicheamicin, maytansine, DM-1, DM-4, netropsin. Other suitable cytotoxic agents include anti-tubulin agents, such as an auristatin, a vinca alkaloid, a podophyllotoxin, a taxane, a baccatin derivative, a cryptophysin, a maytansinoid, a combretastatin, or a dolastatin. Antitubulin agent include dimethylvaline-valinedolaisoleuine-dolaproine-phenylalanine-p-phenylened-iamine (AFP), MMAF, MMAE, auristatin E, vincristine, vinblastine, vindesine, vinorelbine, VP-16, camptothecin, paclitaxel, docetaxel, epothilone A, epothilone B, nocodazole, colchicines, colcimid, estramustine, cemadotin, discodermolide, maytansine, DM-1, DM-4 or eleutherobin.

In one embodiment, an anti-BCMA ADC comprises an anti-BCMA antibody linked to MMAE or MMAF.

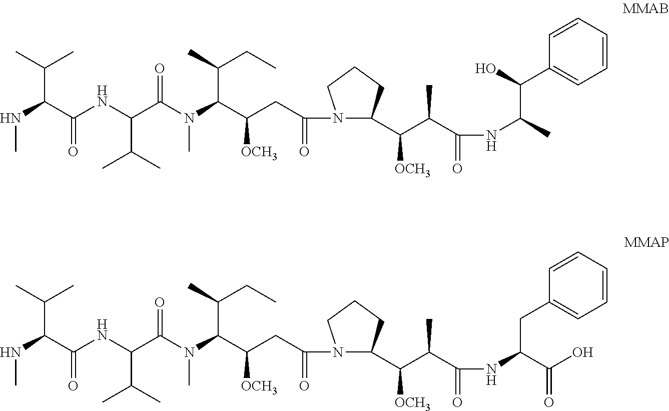 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)C(NC(=O)[C@@H](NC)C(C)C)C(C)C ; CC[C@H](C)[C@@H]([C@@H](CC(=O)N1CCC[C@H]1[C@H](OC)[C@@H](C)C(=O)N[C@@H](Cc1ccccc1)C(=O)O)OC)N(C)C(=O)C(NC(=O)[C@@H](NC)C(C)C)C(C)C]

Exemplary linkers include cleavable and non-cleavable linkers. A cleavable linker may be susceptible to cleavage under intracellular conditions. Suitable cleavable linkers include, for example, a peptide linker cleavable by an intracellular protease, such as lysosomal protease or an endosomal protease. In exemplary embodiments, the linker can be a dipeptide linker, such as a valine-citrulline (val-cit) or a phenylalanine-lysine (phe-lys) linker. Other suitable linkers include, for example, linkers hydrolyzable at a pH of less than 5.5, such as a hydrazone linker. Additional suitable cleavable linkers include, for example, disulfide linkers. Exemplary linkers include 6-maleimidocaproyl (MC), maleimidopropanoyl (MP), valine-citrulline (val-cit), alanine-phenylalanine (ala-phe), p-aminobenzyloxycarbonyl (PAB), N-Succinimidyl 4-(2-pyridylthio)pentanoate (SPP), N-succinimidyl 4-(N-maleimidomethyl)cyclohexane-1 carboxylate (SMCC), and N-Succinimidyl (4-iodo-acetyl) aminobenzoate (STAB).

In one embodiment, a linker may comprise of a thiol-reactive maleimide, a caproyl spacer, dipeptide valine-5 citrulline, a p-aminobenzyloxycarbonyl, a self-immolative fragmenting group, or a protease-resistant maleimidocaproyl.

In another embodiment, an anti-BCMA ADC comprises an anti-BCMA antibody linked to MMAE or MMAF by an MC linker as depicted in the following structures:

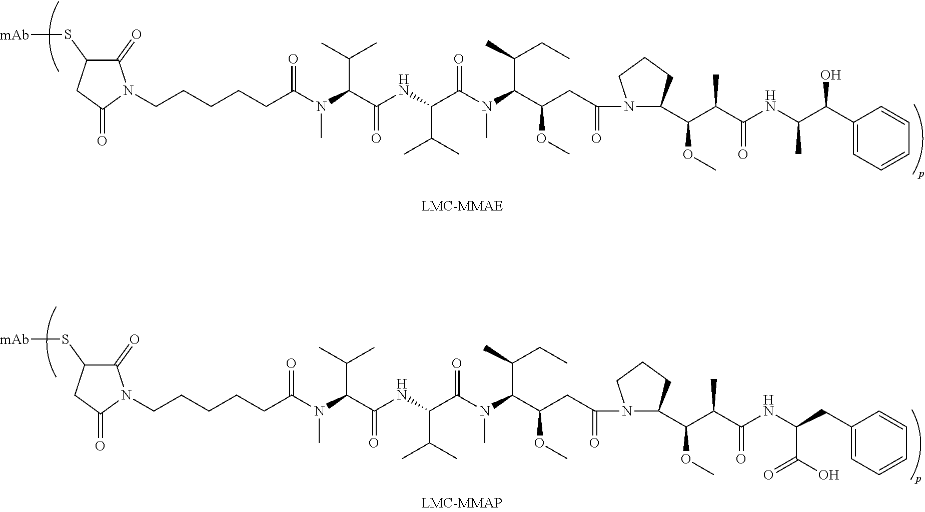

An anti-BCMA ADC described herein may contain any anti-BCMA antibody described herein with any cytotoxic agent described herein.

In one embodiment, an anti-BCMA ADC comprises an anti-BCMA antibody comprising a CDRH1 comprising an amino acid sequence with at least about 90%, 91%, 92%, 93%, 94%, 95%, 96%, 97%, 98%, 99% or 100% sequence identity to the amino acid sequence set forth in SEQ ID NO:1; a CDRH2 comprising an amino acid sequence with at least about 90%, 91%, 92%, 93%, 94%, 95%, 96%, 97%, 98%, 99% or 100% sequence identity to the amino acid sequence set forth in SEQ ID NO:2; a CDRH3 comprising an amino acid sequence with at least about 90%, 91%, 92%, 93%, 94%, 95%, 96%, 97%, 98%, 99% or 100% sequence identity to the amino acid sequence set forth in SEQ ID NO:3; a CDRL1 comprising an amino acid sequence with at least about 90%, 91%, 92%, 93%, 94%, 95%, 96%, 97%, 98%, 99% or 100% sequence identity to the amino acid sequence set forth in SEQ ID NO:4; a CDRL2 comprising an amino acid sequence with at least about 90%, 91%, 92%, 93%, 94%, 95%, 96%, 97%, 98%, 99% or 100% sequence identity to the amino acid sequence set forth in SEQ ID NO:5; and/or a CDRL3 comprising an amino acid sequence with at least about 90%, 91%, 92%, 93%, 94%, 95%, 96%, 97%, 98%, 99% or 100% sequence identity to the amino acid sequence set forth in SEQ ID NO:6; and is conjugated to MMAE or MMAF.

In yet another embodiment, an anti-BCMA ADC comprises an anti-BCMA antibody comprising a CDRH1 with the amino acid sequence set forth in SEQ ID NO:1; a CDRH2 with the amino acid sequence set forth in SEQ ID NO:2; a CDRH3 with the amino acid sequence set forth in SEQ ID NO:3; a CDRL1 with the amino acid sequence set forth in SEQ ID NO:4; a CDRL2 with the amino acid sequence set forth in SEQ ID NO:5; and a CDRL3 with the amino acid sequence set forth in SEQ ID NO:6; and is conjugated to MMAF or MMAE.

In one embodiment, an anti-BCMA ADC comprises an anti-BCMA antibody comprising a V_{H} comprising an amino acid sequence with at least about 90%, 91%, 92%, 93%, 94%, 95%, 96%, 97%, 98%, 99% or 100% sequence identity to the amino acid sequence set forth in SEQ ID NO:7; and/or a V_{L} comprising an amino acid sequence with at least about 90%, 91%, 92%, 93%, 94%, 95%, 96%, 97%, 98%, 99% or 100% sequence identity to the amino acid sequence set forth in SEQ ID NO:8; and is conjugated to MMAE or MMAF.

In yet another embodiment, an anti-BCMA ADC comprises an anti-BCMA antibody comprising a V_{H} with the amino acid sequence set forth in SEQ ID NO:7; and a V_{L} with the amino acid sequence set forth in SEQ ID NO:8; and is conjugated to MMAF or MMAE.

In one embodiment, an anti-BCMA ADC comprises an anti-BCMA antibody comprising a HC comprising an amino acid sequence with at least about 90%, 91%, 92%, 93%, 94%, 95%, 96%, 97%, 98%, 99% or 100% sequence identity to the amino acid sequence set forth in SEQ ID NO:9; and/or a LC comprising an amino acid sequence with at least about 90%, 91%, 92%, 93%, 94%, 95%, 96%, 97%, 98%, 99% or 100% sequence identity to the amino acid sequence set forth in SEQ ID NO:10; and is conjugated to MMAF or MMAE.

In yet another embodiment, an anti-BCMA ADC is belantamab mafodotin comprising an anti-BCMA antibody comprising a HC with the amino acid sequence set forth in SEQ ID NO:9, and a LC with the amino acid sequence set forth in SEQ ID NO:10; and is conjugated to MMAF.

### Preparation and Characterization of ADCs

In certain natural IgG1 molecules comprise 16 disulfide bonds (32 cysteines or sulfhydryl groups). In certain aspects, an antibody can be reduced in such a way that only the four interchain disulfide bonds are reduced and conjugated to a cytotoxic agent, allowing for up to eight sites of attachment for the cytotoxic agent. In other words, the drug load (“DL”), i.e. number of cytotoxic agents per antibody molecule can range from 0 to 8 and are described herein as DL0, DL2 (including DL2a and DL2b), DL4 (including DL4a, DL4b, and DL4c), DL6 (including DL6a and DL6b), and DL8.

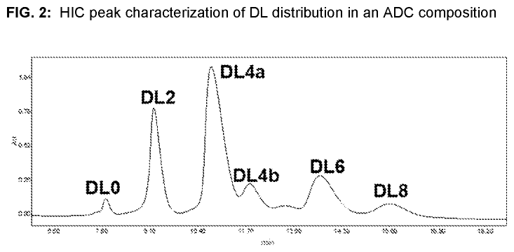

The conjugation process may lead to heterogeneity in drug-antibody attachment for a given ADC composition, varying in both 1) the number of drugs bound to each antibody molecule and 2) the location of the cytotoxic agent. This may lead to an ADC composition with various DL species as exemplified in FIG. 1. The term “ADC composition”, as used herein, refers to a composition containing a heterogeneous mixture of antibody species containing various drug loads (“DL”). (See e.g., FIG. 2). The average drug-antibody ratio of the entire heterogenous ADC composition is referred to herein as “average DAR” or “DAR”. For example, an ADC composition may comprise of mixture of antibody species each with their own DL (some species in the mixture are DL2, some species in the mixture are DL4, some species in the mixture are DL6, and some species in the mixture are DL8) and the average DAR for the entire composition may be about 4.

In another embodiment, the term “percent DL” may be used to describe the percent of a specific DL species within the heterogenous ADC composition (e.g. percent DL2 is about 10% to about 30% of the total heterogenous ADC composition).

In certain aspects of the invention, drugs may be conjugated to antibodies via sulfhydryl groups on the antibody. The sulfhydryl groups can be sulfhydryl groups on cysteine side chains. The cysteine residues can be naturally present in an antibody (e.g., interchain disulfides) or introduced by other means, e.g., mutagenesis. Methods of conjugating drugs to sulfhydryl groups on antibodies are well-known in the art (see, e.g., U.S. Pat. Nos. 7,659,241, 7,498,298, and International Publication No. WO 2011/130613, WO 2014/152199, WO 2015/077605 and Bioconjugate Chem. 2005, 16, 1282-1290). Antibodies are typically reduced prior to conjugation in order to render sulfhydryl groups available for conjugation. Antibodies can be reduced using known conditions in the art. Reducing conditions are those that generally do not cause any substantial denaturation of the antibody and generally do not affect the antigen binding affinity of the antibody. In one aspect of the invention, the reducing agent used in the reduction step is TCEP (tris(2-carboxyethyi)phosphine) and the TCEP is added, e.g., at an excess for 30 minutes at room temperature. For example, 250 μL of a 10 mM solution of TCEP at pH 7.4 will readily reduce the interchain disulfides of 1 to 100 ug of antibody in 30 minutes at room temperature. Other reducing agents and conditions, however, can be used. Examples of reaction conditions include temperatures from 5° C. to 37° C. over a pH range of 5 to 8.

Various methods exist, and are known to those skilled in the art, for calculating the percent DL species and/or average DAR in an ADC composition. For example, heterogeneity of cysteine-linked ADCs is typically measured by hydrophobic interaction chromatography (HIC) which separates DL species based on the number of drugs loaded. LC-MS assays have also been developed to asses DL distribution. Exemplary methods for calculating the drug load distribution in an ADC composition can be found, for example, in Journal of Chromatography B 1060 (2017) 182-189.

For example, DL0 has no drug load on the antibody. For example, DL2 has a drug load of two. In one embodiment, the conjugation sites for DL2 are LC C214 and HC 224. For example, DL4 has a drug load of four. In one embodiment, the conjugation sites for DL4a are LC C214, HC 224, LC C214 and HC 224. In one embodiment, the conjugation sites for DL4b are LC C230, HC 233, LC C230 and HC 233. For example, DL6 has a drug load of six. In one embodiment, the conjugation sites for DL6 are LC C214, HC 224, LC C230, HC 233, LC C230 and HC 233. For example, DL8 has a drug load of eight. In one embodiment, the conjugation sites for DL8 are LC C214, HC 224, LC C214, HC 224, LC C230, HC 233, LC C230 and HC 233.

In one embodiment, the percent of a specific DL species (e.g. percent DL0, percent DL2, percent DL4a, percent DL4b, percent DL6, percent DL8) may be determined by separating individual DL species using hydrophobic interaction chromatography (HIC), calculating the area under the curve for each DL peak, and dividing each DL peak by the total area under the curve for all DL species combined. In one embodiment, the average DAR can be calculated from the area under the curve of each DL species using the following formula:

$$\% DL {Component}_{X} = \frac{{A}_{X}}{{A}_{0} + {A}_{1} + {A}_{2} + {A}_{3} + {A}_{4 a} + {A}_{4 b} + {A}_{5} + {A}_{6} + {A}_{8} + {A}_{10}} \times 100$$

$$DAR = \frac{\begin{matrix}( {A}_{1} \times 2 ) + ( {A}_{2} \times 2 ) + ( {A}_{3} \times 2 ) + ( {A}_{4 a} \times 4 ) + \\ ( {A}_{4 b} \times 4 ) + ( {A}_{5} \times 6 ) + ( {A}_{6} \times 6 ) + ( {A}_{8} \times 8 ) \times ( {A}_{10} \times 8 )\end{matrix}}{{A}_{0} + {A}_{1} + {A}_{2} + {A}_{3} + {A}_{4 a} + {A}_{4 b} + {A}_{5} + {A}_{6} + {A}_{8} + {A}_{10}}$$

$$Where : \\ {A}_{X} = Peak area of loading X peak \\ X = {A}_{0} , {A}_{1} , {A}_{2} , {A}_{3} , {A}_{4 a} , {A}_{4 b} , {A}_{5} , {A}_{6} , {A}_{8} , and {A}_{10} \\ {A}_{0} , {A}_{1} , {A}_{2} , {A}_{3} , {A}_{4 a} , {A}_{4 b} , {A}_{5} , {A}_{6} , {A}_{8} , and {A}_{10} = is the peak of DL 0 , DL 1 , DL 2 , DL 3 , \\ DL 4 a , DL 4 b , DL 5 , DL 6 , DL 8 and DL 10 peaks \\ ( only including peaks \geq DL ( 0.08 \% ) )$$

In one embodiment, the percent of a specific DAR sub-species (e.g. Percent of DL2a in total DL2) is determined by collection of a specific DL species using a combination of analytical techniques that could include HIC, non-reducing separation methods, and mass spectrometric techniques.

In one embodiment, an anti-BCMA ADC composition has an average DAR of about 2 to about 7, about 2 to about 6, about 2.1 to about 5.7, about 2.1 to about 5.0, about 2.1 to about 4.6, about 2.1 to about 4.1, about 2.1 to about 3.5, about 2.1 to about 3.0, about 3.0 to about 5.7, about 3.0 to about 5.0, about 3.0 to about 4.6, about 3.0 to about 4.1, about 3.0 to about 3.5, about 3.5 to about 5.7, about 3.5 to about 5.0, about 3.5 to about 4.6, about 3.5 to about 4.1, about 3.8 to about 4.5, about 4.1 to about 5.7, about 4.1 to about 5.0, about 4.1 to about 4.6, about 4.6 to about 5.7, about 4.6 to about 5.0, about 5.0 to about 5.7, about 2.1, about 3.0, about 3.5, about 4.1, about 4.6, about 5.0, or about 5.7

In another embodiment, a composition comprises an anti-BCMA ADC, wherein the average DAR is about 2.1 to about 5.7, about 3.4 to about 4.6, about 3.8 to about 4.5, or about 4.

In one embodiment, a composition comprises an anti-BCMA ADC, wherein the antibody comprises a CDRH1 with the amino acid sequence set forth in SEQ ID NO:1, a CDRH2 with the amino acid sequence set forth in SEQ ID NO:2, a CDRH3 with the amino acid sequence set forth in SEQ ID NO:3, a CDRL1 with the amino acid sequence set forth in SEQ ID NO:4, a CDRL2 with the amino acid sequence set forth in SEQ ID NO:5, and a CDRL3 with the amino acid sequence set forth in SEQ ID NO:6; wherein the cytotoxic agent is MMAE or MMAF; and wherein the average DAR is about 2 to about 6, about 2.1 to about 5.7, about 3.4 to about 4.6, or about 3.8 to about 4.5.

In one embodiment, a composition comprises an anti-BCMA ADC, wherein the antibody comprises a V_{H} with the amino acid sequence set forth in SEQ ID NO:7, and a V_{L} with the amino acid sequence set forth in SEQ ID NO:8; wherein the cytotoxic agent is MMAE or MMAF; and wherein the average DAR is about 2 to about 6, about 2.1 to about 5.7, about 3.4 to about 4.6, or about 3.8 to about 4.5.

In one embodiment, the composition comprises belantamab mafodotin, wherein the average DAR is about 2 to about 6, about 2.1 to about 5.7, about 3.4 to about 4.6, or about 3.8 to about 4.5.

In one embodiment, percent DL0 species in an anti-BCMA ADC composition is about 10% or less, about 5% or less, about 1% to about 10%, about 1% to about 5%, or about 2.8% to about 4.7%.

In one embodiment, percent DL2 species in an anti-BCMA ADC composition is at least about 10%, at least about 15%, about 15.8% to about 26.3%, about 15% to about 27%, about 15% to about 32%, or about 10% to about 40%.

In one embodiment, percent DL4a species in an anti-BCMA ADC composition is at least about 30%, at least about 35%, about 35.5% to about 37.9%, about 35% to about 38%, about 30% to about 40%, or about 20% to about 50%. In another embodiment, percent DL4a species is the predominant species in the anti-BCMA ADC composition and comprises about ≥30%, ≥40%, ≥50%, ≥60%, ≥70%, ≥80%, or ≥90% of the all species combined.

In one embodiment, percent DL4b species in an anti-BCMA ADC composition is at least about 5%, at least about 7%, about 7.1% to about 8.5%, about 7% to about 9%, about 5% to about 10%, or about 1% to about 15%.

In one embodiment, percent DL6 species in an anti-BCMA ADC composition is at least about 10%, at least about 14%, about 14.0% to about 19.1%, about 14% to about 20%, about 10% to about 20%, or about 5% to about 30%.

In one embodiment, percent DL8 species in an anti-BCMA ADC composition is at least about 1%, at least about 6%, about 6.0% to about 12.0%, about 4% to about 15%, or about 1% to about 20%.

In one embodiment, a composition comprises an anti-BCMA ADC, wherein percent DL2 is about 15% to about 27% or about 15% to about 32%, percent DL4a is about 35% to about 38% or about 30% to about 40%, percent DL4b is about 7% to about 9% or about 5% to about 10%, percent DL6 is about 14% to about 20% or about 10% to about 20%, and/or DL8 is about 6.0% to about 12.0% or about 4% to about 15%.

In one embodiment, a composition comprises an anti-BCMA ADC, wherein the antibody comprises a CDRH1 with the amino acid sequence set forth in SEQ ID NO:1, a CDRH2 with the amino acid sequence set forth in SEQ ID NO:2, a CDRH3 with the amino acid sequence set forth in SEQ ID NO:3, a CDRL1 with the amino acid sequence set forth in SEQ ID NO:4, a CDRL2 with the amino acid sequence set forth in SEQ ID NO:5, and a CDRL3 with the amino acid sequence set forth in SEQ ID NO:6; wherein the cytotoxic agent is MMAE or MMAF; and wherein percent DL2 is about 15% to about 27% or about 15% to about 32%, percent DL4a is about 35% to about 38% or about 30% to about 40%, percent DL4b is about 7% to about 9% or about 5% to about 10%, percent DL6 is about 14% to about 20% or about 10% to about 20%, and/or DL8 is about 6.0% to about 12.0% or about 4% to about 15%.

In one embodiment, a composition comprises an anti-BCMA ADC, wherein the antibody comprises a V_{H} with the amino acid sequence set forth in SEQ ID NO:7, and a V_{L} with the amino acid sequence set forth in SEQ ID NO:8; wherein the cytotoxic agent is MMAE or MMAF; and wherein percent DL2 is about 15% to about 27% or about 15% to about 32%, percent DL4a is about 35% to about 38% or about 30% to about 40%, percent DL4b is about 7% to about 9% or about 5% to about 10%, percent DL6 is about 14% to about 20% or about 10% to about 20%, and/or DL8 is about 6.0% to about 12.0% or about 4% to about 15%.

In one embodiment, a composition comprises belantamab mafodotin, wherein percent DL2 is about 15% to about 27% or about 15% to about 32%, percent DL4a is about 35% to about 38% or about 30% to about 40%, percent DL4b is about 7% to about 9% or about 5% to about 10%, percent DL6 is about 14% to about 20% or about 10% to about 20%, and/or DL8 is about 6.0% to about 12.0% or about 4% to about 15%.

The term “undesired DAR species”, as used herein, refers to any DAR species which is not desired in the final composition and which may have a negative impact on certain properties (e.g. target binding, efficacy, safety, etc.) of the final therapeutic product. In one embodiment, an undesired DAR species is DL0 i.e., antibody not bound with cytotoxic agent after the conjugation process. In one embodiment, percent DL0 in the ADC composition is less than or equal to about 15%, about 14%, about 13%, about 12%, about 11%, about 10%, about 9%, about 8%, about 7%, about 6%, about 5%, about 4%, about 3%, about 2%, about 1%, or about 0.5%. In another embodiment, percent DL0 in the ADC composition is about 1% to about 10%, about 2% to about 5%, or about 2.0% to about 4.8%.

In one embodiment, a composition comprises an anti-BCMA ADC, wherein the antibody comprises a CDRH1 with the amino acid sequence set forth in SEQ ID NO:1, a CDRH2 with the amino acid sequence set forth in SEQ ID NO:2, a CDRH3 with the amino acid sequence set forth in SEQ ID NO:3, a CDRL1 with the amino acid sequence set forth in SEQ ID NO:4, a CDRL2 with the amino acid sequence set forth in SEQ ID NO:5, and a CDRL3 with the amino acid sequence set forth in SEQ ID NO:6; wherein the cytotoxic agent is MMAE or MMAF; and wherein percent DL0 is less than or equal to about 10% or about 5%_{.}

In one embodiment, a composition comprises an anti-BCMA ADC, wherein the antibody comprises a V_{H} with the amino acid sequence set forth in SEQ ID NO:7, and a V_{L} with the amino acid sequence set forth in SEQ ID NO:8; wherein the cytotoxic agent is MMAE or MMAF; and wherein percent DL0 is less than or equal to about 10% or about 5%.

In one embodiment, a composition comprises belantamab mafodotin, and wherein percent DL0 is less than or equal to about 10% or about 5%.

In one embodiment, a composition comprises belantamab mafodotin, wherein percent DL0 is less than or equal to about 10% or about 5%, percent DL2 is about 15% to about 27% or about 15% to about 32%, percent DL4a is about 35% to about 38% or about 30% to about 40%, percent DL4b is about 7% to about 9% or about 5% to about 10%, percent DL6 is about 14% to about 20% or about 10% to about 20%, and/or DL8 is about 6.0% to about 12.0% or about 4% to about 15%.

In certain embodiments, average DAR or percent DL impacts cell growth inhibition and/or tumor volume. In certain embodiments, average DAR does not impact cell growth inhibition and/or tumor volume. In another embodiment, as average DAR or percent DL increases, cell growth inhibition increases and/or tumor volumes decreases. In yet another embodiment, a composition comprises belantamab mafodotin, and as average DAR or percent DL of the composition increases, cancer cell growth inhibition increases and/or tumor volumes decreases.

Cell growth inhibition relative potency can be determined by measuring cell viability of a cell line (e.g. multiple myeloma cell line) after incubation with a composition described herein (e.g. belantamab mafodotin). The cell viability can be measured using a cell viability assay known to those skilled in the art. The dose response (half maximal effective concentration or EC50) can be generated using a nonlinear regression logistic model. The ratio of the EC50 of a reference standard to the EC50 of the sample containing the composition can be calculated to determine the relative potency

In one embodiment a composition has a cell growth inhibition relative potency of about 0.5 to about 1.3, or about 0.8 to about 1.1. In another embodiment, a composition comprises an average DAR of about 2.1 to about 5.7, and has a cell growth inhibition relative potency of about 0.5 to about 1.3. In another embodiment, a composition comprises an average DAR of about 3.0 to about 5.0 or about 3.5 to about 4.6, and has a cell growth inhibition relative potency of about 0.8 to about 1.1. In another embodiment, a composition comprises belantamab mafodotin with an average DAR of about 3.0 to about 5.0 or about 3.5 to about 4.6, and has a cell growth inhibition relative potency of about 0.8 to about 1.1.

In certain embodiments, average DAR or percent DL impacts ADCC activity. In another embodiment, average DAR or percent DL does not impact ADCC activity. In yet another embodiment, a composition comprises belantamab mafodotin, and average DAR or percent DL impacts ADCC activity. In yet another embodiment, the composition comprises belantamab mafodotin, and average DAR or percent DL does not impact ADCC activity.

ADCC activity relative potency can be measured, for example, by incubating the belantamab mafodotin, cells (e.g. multiple myeloma cells), and NK-cells (effector cells). Without being bound by theory, belantamab mafodotin binds to BCMA expressed on the cell surface, and the Fc region of the antibody binds to FcγRIIIa on the effector cells through their FcγRIIIa receptor. Engagement of these receptors on the surface of the effector cells results in the synthesis and secretion of cytokines (IFNg), and release of granules (perforin and granzymes) that enter into the cytoplasm of the target cells. The granzymes initiate a signaling event within the target cells that cause the death of these cells by apoptosis. The source of NK-cells can be Peripheral Blood Mononuclear Cells (PBMC), which may be isolated from human whole blood. BATDA (bis-(acetoxymethyl) 2,2′:6′,2″-terpyridine-6,6″-dicarboxylate) can then be added to penetrate the target cell membrane to label the cell. After cytolysis, this ligand can be combined with a DELFIA Europium Solution to form a highly fluorescent and stable chelate (EuTDA). The measured signal correlates directly with the amount of lysed cells. The ADCC activity can then be reported as a ratio of sample EC50 value versus that of a reference standard.

In one embodiment a composition has an ADCC activity relative potency of about 0.70 to about 1.30, or about 0.8 to about 1.1. In another embodiment, a composition comprises an average DAR of about 2.1 to about 5.7, and has an ADCC activity relative potency of about 0.5 to about 1.3. In another embodiment, a composition comprises an average DAR of about 3.0 to about 5.0 or about 3.5 to about 4.6, and has an ADCC activity relative potency of about 0.8 to about 1.1. In another embodiment, a composition comprises belantamab mafodotin with an average DAR of about 3.0 to about 5.0 or about 3.5 to about 4.6, and has an ADCC activity relative potency of about 0.8 to about 1.1.

In certain embodiments, average DAR or percent DL impacts binding to BCMA. In another embodiment, average DAR or percent DL does not impact binding to BCMA. In yet another embodiment, a composition comprises belantamab mafodotin, and average DAR or percent DL impacts binding to BCMA. In yet another embodiment, a composition comprises belantamab mafodotin, and average DAR or percent DL does not impact binding to BCMA. In one embodiment, average DAR or percent DL may weaken binding to BCMA.

In one embodiment, a composition has ≥70%, ≥75%, ≥80%, ≥85%, ≥90%, ≥95% relative BCMA specific antigen binding. In another embodiment, a composition comprises belantamab mafodotin and has a relative BCMA specific antigen binding of greater than 85% or greater than 90%. In another embodiment, a composition comprises belantamab mafodotin, has an average DAR of about 2.1 to about 5.7 or about 3.0 to about 5.0 or about 3.5 to about 4.6, and has a relative BCMA specific antigen binding of greater than 85% or greater than 90%.

In certain embodiments, average DAR or percent DL impacts binding to FcγRIIIa. In another embodiment, average DAR or percent DL does not impact binding to FcγRIIIa. In yet another embodiment, a composition comprises belantamab mafodotin, and the average DAR or percent DL impacts binding to FcγRIIIa. In yet another embodiment, a composition comprises belantamab mafodotin, and average DAR or percent DL does not impact binding to FcγRIIIa. In one embodiment, average DAR or percent DL may weaken binding to FcγRIIIa.

In one embodiment, a composition has ≥70%, ≥75%, ≥80%, ≥85%, ≥90%, ≥95% relative FcγRIIIa binding. In another embodiment, a composition comprises belantamab mafodotin and has a relative FcγRIIIa binding of greater than 85% or greater than 90%. In another embodiment, a composition comprises belantamab mafodotin, has an average DAR of about 2.1 to about 5.7 or about 3.0 to about 5.0 or about 3.5 to about 4.6, and has a relative FcγRIIIa binding of greater than 85% or greater than 90%.

Among other methods known to those skilled in the art, the binding of BCMA and FcγRIIIa by an anti-BCMA antigen binding protein, e.g., belantamab mafodotin, can measured using surface plasmon resonance (SPR). In one example, belantamab mafodotin is injected and captured by protein A immobilized on a CM5 sensor chip. BCMA is then injected and bound to the captured belantamab mafodotin. Next, FcγRIIIa is injected and bound to the captured belantamab mafodotin. The functional concentrations of belantamab mafodotin binding to BCMA and FcγRIIIa can be calculated from a reference standard curve and reported as the BCMA or FcγRIIIa binding concentration, respectively. The total belantamab mafodotin concentration of the sample can be pre-determined by absorbance at 280 nm. The specific binding activity (%) can be calculated by dividing the BCMA or FcγRIIIa binding concentration by the absorbance at, e.g., 280 nm concentration.

In certain embodiments, average DAR or percent DL impacts binding to FcRn. In another embodiment, average DAR or percent DL does not impact binding to FcRn. In yet another embodiment, a composition comprises belantamab mafodotin, and the average DAR or percent DL impacts binding to FcRn. In yet another embodiment, a composition comprises belantamab mafodotin, and average DAR or percent DL does not impact binding to FcRn. In one embodiment, average DAR or percent DL may weaken binding to FcRn.

The binding of Neonatal Fc Receptor (FcRn) to an anti-BCMA antigen binding protein, e.g., belantamab mafodotin, can measured using surface plasmon resonance (SPR). Belantamab mafodotin can be captured by FcRn, which is immobilized on a nitrilotriacetic acid (NTA) sensor chip. The FcRn binding concentration of the sample can be determined by interpolation of the binding response on a calibration curve. Specific binding activity (%) is calculated by dividing the FcRn binding concentration by the total protein concentration.

When an anti-BCMA antigen binding protein comprises belantamab mafodotin, the SPR methods described herein for specific antigen binding, FcγRIIIa and FcRn binding may use a reference standard of belantamab or belantamab mafodotin. The belantamab or belantamab mafodotin reference standard can be used in assays to obtain system suitability and sample comparability data, to ensure methods are performing appropriately. The reference standard can allow the establishment of a calibration curve and concentrations of the samples are interpolated from the curve. For example, a reference standard may be a composition comprising a heavy chain amino acid sequence of SEQ ID NO:9 and a light chain amino acid sequence of SEQ ID NO:10 and comprises a known DL and/or average DAR content.

Exemplary reference standards may include samples of belantamab mafodotin with known components/amounts of DL species and/or average DAR.

### Pharmaceutical Compositions

A composition described herein can be in the form of a pharmaceutical composition. A “pharmaceutical composition” may comprise a composition described herein (i.e. active ingredient), and one or more pharmaceutically acceptable excipients. The excipient(s) must be acceptable in the sense of being compatible with the other ingredients of the formulation, capable of pharmaceutical formulation, not deleterious to the recipient thereof, and/or do not interfere with the efficacy of the active ingredient.

As used herein, “pharmaceutically acceptable excipient” may include any and all solvents, diluents, carriers, dispersion media, coatings, antibacterial and antifungal agents, isotonic and/or absorption delaying agents. Examples of pharmaceutically acceptable excipients include one or more of buffering agents, water, saline, phosphate buffered saline, dextrose, glycerol, ethanol and the like, as well as combinations thereof. In many cases, it will be preferable to include isotonic agents, for example, polyol, sugars, polyalcohols such as mannitol, sorbitol, or sodium chloride in the composition. preservatives; co-solvents; antioxidants including ascorbic acid and methionine; chelating agents such as EDTA; metal complexes (e.g., Zn2⁺-protein complexes); biodegradable polymers; and/or salt-forming counterions such as sodium or potassium.

The precise nature of the excipient or other material may depend on the route of administration, which may be, for example, oral, rectal, nasal, topical (including buccal and sublingual), vaginal, parenteral (including subcutaneous, intramuscular, intravenous, intradermal, intrathecal, and epidural), and intratumorally. It will be appreciated that the preferred excipient may vary with, for example, the condition of the recipient and the disease to be treated.

A mixture of excipients and concentrations of each together form a “pharmaceutical formulation” (or “formulation”). The formulation may be in liquid form or lyophilized form. A composition in a liquid formulation may be filled into containers and frozen. In certain embodiments, aliquots of the frozen formulation comprising the composition may be lyophilized. Lyophilizate may be reconstituted by the addition of water or other aqueous solution to produce a reconstituted formulation comprising the composition.

In some embodiments, an anti-BMCA antigen binding protein is present in a formulation at a concentration of at least about 10 mg/mL or at least about 20 mg/mL. In some embodiments, an anti-BMCA antigen binding protein is present in a formulation at a concentration of between about 20 mg/mL to about 100 mg/mL, or about 20 mg/mL to about 60 mg/mL. In certain embodiments, the concentration of an anti-BCMA antigen binding protein in the formulation is about 20 mg/mL, about 25 mg/mL, about 50 mg/mL, about 60 mg/mL, or about 100 mg/mL. In one embodiment, an anti-BMCA antigen binding protein is present in a liquid formulation at a concentration of about 20 mg/mL or about 25 mg/mL. In another embodiment, an anti-BMCA antigen binding protein is present in a lyophilized formulation at a concentration of about 50 mg/mL or about 60 mg/mL. In yet another embodiment, the anti-BMCA antigen binding protein is present in a reconstituted formulation at a concentration of about 50 mg/mL.

In certain embodiments, a buffering agent is a citrate buffer. Citrate buffer can be achieved, for example, by the use of a conjugate acid/conjugate base system (sodium citrate/citric acid) or by HCl titration of a sodium citrate solution. In certain embodiments, the concentration of a citrate buffer is about 10 mM to about 30 mM. In preferred embodiments, the concentration of a citrate buffer is 25 mM. In some embodiments, a buffering agent is a histidine buffer at a concentration from about 5 mM to about 35 mM.

A buffering agent may be used to help maintain preferred pH ranges. In certain embodiments, the pH of a formulation is about 5.5 to about 7 or about 5.9 to about 6.5, preferably pH 6.2.

In some embodiments, a formulation comprises a polyol. In some embodiments, a polyol is a sugar, and preferably a non-reducing sugar. In some embodiments, a non-reducing sugar is trehalose. In some embodiments, the formulation comprises trehalose in the range from about from about 120 mM to about 240 mM. In yet another embodiment, the formulation comprises trehalose at about 200 mM.

In one embodiment, a formulation comprises a chelating agent. In another embodiment, a chelating agent is EDTA. In certain embodiments, the formulation comprises EDTA at a concentration of 0.01 mM to about 0.1 mM. In yet another embodiment, the formulation comprises EDTA at a concentration of 0.05 mM.

In some embodiments, a formulation comprises a surfactant. “Surfactants” are surface active agents that can exert their effect at surfaces of solid-solid, solid-liquid, liquid-liquid, and liquid-air interfaces because of their chemical composition, containing both hydrophilic and hydrophobic groups. Surfactants may reduce the concentration of proteins in dilute solutions at the air-water and/or water-solid interfaces where proteins can be adsorbed and potentially aggregated. Surfactants can bind to hydrophobic interfaces in protein formulations. Some parentally acceptable nonionic surfactants comprise either polysorbate or polyether groups. Polysorbate 20 and 80 are suitable surfactant stabilizers in formulations of the invention. In some embodiments, a formulation comprises polysorbate 20 or polysorbate 80 at about 0.01% to about 0.05%. In yet another embodiment, a formulation comprises polysorbate 20 or polysorbate 80 at about 0.02%. In a preferred embodiment, a formulation comprises polysorbate 80 at about 0.02%.

One aspect of the invention is drawn to a formulation that comprises from about 20 mg/mL to about 100 mg/mL of an anti-BCMA antigen-binding protein, from about 10 mM to about 25 mM of a buffering agent, from about 120 mM to about 240 mM of a polyol, and a pH in the range of 5.5 to 6.5.

In one embodiment, a formulation comprises an anti-BCMA antigen binding protein at about 20 mg/mL to about 60 mg/mL, citrate buffer at about 10 mM to about 30 mM, trehalose at about 120 mM to about 240 mM, EDTA at about 0.01 mM to about 0.1 mM, polysorbate 20 or polysorbate 80 at about 0.01% to about 0.05%, at a pH of about 5.9 to about 6.5.

In one embodiment, a composition comprises an antibody in a formulation, wherein the antibody comprises a CDRH1 with the amino acid sequence set forth in SEQ ID NO:1, a CDRH2 with the amino acid sequence set forth in SEQ ID NO:2, a CDRH3 with the amino acid sequence set forth in SEQ ID NO:3, a CDRL1 with the amino acid sequence set forth in SEQ ID NO:4, a CDRL2 with the amino acid sequence set forth in SEQ ID NO:5, and a CDRL3 with the amino acid sequence set forth in SEQ ID NO:6; and wherein the formulation comprises the antibody at about 20 mg/mL to about 60 mg/mL, citrate buffer at about 10 mM to about 30 mM, trehalose at about 120 mM to about 240 mM, EDTA at about 0.01 mM to about 0.1 mM, polysorbate 20 or polysorbate 80 at about 0.01% to about 0.05%, at a pH of about 5.9 to about 6.5.

In one embodiment, a composition comprises an antibody in a formulation, wherein the antibody comprises a V_{H} with the amino acid sequence set forth in SEQ ID NO:7, and a V_{L} with the amino acid sequence set forth in SEQ ID NO:8; and wherein the formulation comprises the antibody at about 20 mg/mL to about 60 mg/mL, citrate buffer at about 10 mM to about 30 mM, trehalose at about 120 mM to about 240 mM, EDTA at about 0.01 mM to about 0.1 mM, polysorbate 20 or polysorbate 80 at about 0.01% to about 0.05%, at a pH of about 5.9 to about 6.5.

In one embodiment, a composition comprises an antibody in a formulation, wherein the antibody is belantamab; and wherein the formulation comprises belantamab at about 20 mg/mL to about 60 mg/mL, citrate buffer at about 10 mM to about 30 mM, trehalose at about 120 mM to about 240 mM, EDTA at about 0.01 mM to about 0.1 mM, polysorbate 20 or polysorbate 80 at about 0.01% to about 0.05%, at a pH of about 5.9 to about 6.5.

In one embodiment, a composition comprises an ADC in a formulation, wherein the antibody comprises a CDRH1 with the amino acid sequence set forth in SEQ ID NO:1, a CDRH2 with the amino acid sequence set forth in SEQ ID NO:2, a CDRH3 with the amino acid sequence set forth in SEQ ID NO:3, a CDRL1 with the amino acid sequence set forth in SEQ ID NO:4, a CDRL2 with the amino acid sequence set forth in SEQ ID NO:5, and a CDRL3 with the amino acid sequence set forth in SEQ ID NO:6; wherein the cytotoxin is MMAE or MMAF; and wherein the formulation comprises the ADC at about 20 mg/mL to about 60 mg/mL, citrate buffer at about 10 mM to about 30 mM, trehalose at about 120 mM to about 240 mM, EDTA at about 0.01 mM to about 0.1 mM, polysorbate 20 or polysorbate 80 at about 0.01% to about 0.05%, at a pH of about 5.9 to about 6.5.

In one embodiment, a composition comprises an ADC in a formulation, wherein the antibody comprises a V_{H} with the amino acid sequence set forth in SEQ ID NO:7, and a V_{L} with the amino acid sequence set forth in SEQ ID NO:8; wherein the cytotoxin is MMAF or MMAE; and wherein the formulation comprises the ADC at about 20 mg/mL to about 60 mg/mL, citrate buffer at about 10 mM to about 30 mM, trehalose at about 120 mM to about 240 mM, EDTA at about 0.01 mM to about 0.1 mM, polysorbate 20 or polysorbate 80 at about 0.01% to about 0.05%, at a pH of about 5.9 to about 6.5.

In one embodiment, a composition comprises an ADC in a formulation, wherein the ADC is belantamab mafodotin; and wherein the formulation comprises belantamab mafodotin at about 20 mg/mL to about 60 mg/mL, citrate buffer at about 10 mM to about 30 mM, trehalose at about 120 mM to about 240 mM, EDTA at about 0.01 mM to about 0.1 mM, polysorbate 20 or polysorbate 80 at about 0.01% to about 0.05%, at a pH of about 5.9 to about 6.5.

In one embodiment, a composition comprises belantamab mafodotin in a formulation comprising about 20 mg/mL, about 25 mg/mL, about 50 mg/mL, or 60 mg/mL belantamab mafodotin, 25 mM citrate buffer, 200 mM trehalose, 0.05 mM disodium EDTA, 0.02% polysorbate or 80 polysorbate 80 at a pH of about 5.9 to about 6.5.

A “stable” formulation is one in which the protein therein essentially retains its physical and/or chemical stability during manufacturing, transport, storage, and administration. Stability can be measured at a selected temperature for a selected time period. For example, for a product stored at a recommended temperature of 2° C. to 8° C., the formulation is stable at room temperature, about 30° C., or at 40° C., for at least 1 month and/or stable at about 2 to 8° C. for at least 1 year and preferably for at least 2 years. For example, the extent of aggregation during storage can be used as an indicator of protein stability. Thus, a “stable” formulation may be one wherein, for example, less than about 10% and preferably less than about 5% of the protein is present as an aggregate in the formulation. Various analytical techniques for measuring protein stability are available in the art and are reviewed, for example, in Peptide and Protein Drug Delivery, 247-301, Vincent Lee Ed., Marcel Dekker, Inc., New York, N.Y., Pubs. (1991) and Jones, A. Adv. Drug Delivery Rev. 10: 29-90 (1993).

In certain aspects of the invention, a formulation allows the composition to remain stable to freezing, thawing, and/or mixing.

In yet another aspect, the present invention is directed to an article of manufacture, e.g., a kit, comprising a container holding a composition in a formulation described herein. In one aspect there is provided an injection device comprising the formulation. The injection device may comprise a pen injector device or an autoinjector device. In one embodiment, the formulation is contained in a prefilled syringe.

### Methods of Treatment and Compositions for Use

It is an object of the present invention to provide a therapeutic approach to the treatment of B-cell related disorders or diseases, such as antibody-mediated or plasma cell mediated diseases, or plasma cell malignancies (e.g. cancers such as Multiple Myeloma), or other disease that may be treated by an anti-BCMA antigen binding protein. In particular it is an object of the present invention to provide compositions comprising an anti-BCMA antigen binding proteins, for example, anti-BCMA antibodies, that specifically bind to BCMA (e.g. human BCMA) and modulate (i.e. inhibit or block) the interaction between BCMA and its ligands such as BAFF and/or APRIL in the treatment of diseases and disorders responsive to modulation of that interaction.

In another aspect of the present invention, there is provided a method of treating a subject (e.g. human patient) afflicted with a B-cell related disorders or diseases, such as antibody-mediated or plasma cell mediated diseases, or plasma cell malignancies (e.g. cancers such as multiple myeloma), such method comprises the step of administering to said subject a therapeutically effective amount of an anti-BCMA antigen binding protein composition described herein.

In yet another embodiment, present invention provides for a method of treating a cancer patient, which method comprises the step of administering to said patient a therapeutically effective amount of an anti-BCMA antigen binding protein composition described herein.

As used herein, the terms “cancer,” and “tumor” are used interchangeably and, in either the singular or plural form, refer to cells that have undergone a transformation, such as malignant transformation, that makes them pathological to the host organism. Primary cancer cells can be readily distinguished from non-cancerous cells by well-established techniques, particularly histological examination. The definition of a cancer cell, as used herein, includes not only a primary cancer cell, but any cell derived from a cancer cell ancestor. This includes metastasized cancer cells, and in vitro cultures and cell lines derived from cancer cells. When referring to a type of cancer that normally manifests as a solid tumor, a “clinically detectable” tumor is one that is detectable on the basis of tumor mass; e.g., by procedures such as computed tomography (CT) scan, magnetic resonance imaging (MRI), X-ray, ultrasound or palpation on physical examination, and/or which is detectable because of the expression of one or more cancer-specific antigens in a sample obtainable from a patient. Tumors may be a hematopoietic (or hematologic or hematological or blood-related) cancer, for example, cancers derived from blood cells or immune cells, which may be referred to as “liquid tumors.” Specific examples of clinical conditions based on hematologic tumors include leukemias such as chronic myelocytic leukemia, acute myelocytic leukemia, chronic lymphocytic leukemia and acute lymphocytic leukemia; plasma cell malignancies such as multiple myeloma, MGUS and Waldenstrom's macroglobulinemia; lymphomas such as non-Hodgkin's lymphoma, Hodgkin's lymphoma; and the like.

The cancer may be any in which an abnormal number of blast cells or unwanted cell proliferation is present or that is diagnosed as a hematological cancer, including both lymphoid and myeloid malignancies. Myeloid malignancies include, but are not limited to, acute myeloid (or myelocytic or myelogenous or myeloblastic) leukemia (undifferentiated or differentiated), acute promyeloid (or promyelocytic or promyelogenous or promyeloblastic) leukemia, acute myelomonocytic (or myelomonoblastic) leukemia, acute monocytic (or monoblastic) leukemia, erythroleukemia and megakaryocytic (or megakaryoblastic) leukemia. These leukemias may be referred together as acute myeloid (or myelocytic or myelogenous) leukemia (AML). Myeloid malignancies also include myeloproliferative disorders (MPD) which include, but are not limited to, chronic myelogenous (or myeloid) leukemia (CML), chronic myelomonocytic leukemia (CMML), essential thrombocythemia (or thrombocytosis), and polcythemia vera (PCV). Myeloid malignancies also include myelodysplasia (or myelodysplastic syndrome or MDS), which may be referred to as refractory anemia (RA), refractory anemia with excess blasts (RAEB), and refractory anemia with excess blasts in transformation (RAEBT); as well as myelofibrosis (MFS) with or without agnogenic myeloid metaplasia, among others.

Hematopoietic cancers also include lymphoid malignancies, which may affect the lymph nodes, spleens, bone marrow, peripheral blood, and/or extranodal sites. Lymphoid cancers include B-cell malignancies, which include, but are not limited to, B-cell non-Hodgkin's lymphomas (B-NHLs). B-NHLs may be indolent (or low-grade), intermediate-grade (or aggressive) or high-grade (very aggressive). Indolent B-cell lymphomas include follicular lymphoma (FL); small lymphocytic lymphoma (SLL); marginal zone lymphoma (MZL) including nodal MZL, extranodal MZL, splenic MZL and splenic MZL with villous lymphocytes; lymphoplasmacytic lymphoma (LPL); and mucosa-associated-lymphoid tissue (MALT or extranodal marginal zone) lymphoma. Intermediate-grade B-NHLs include mantle cell lymphoma (MCL) with or without leukemic involvement, diffuse large cell lymphoma (DLBCL), follicular large cell (or grade 3 or grade 3B) lymphoma, and primary mediastinal lymphoma (PML). High-grade B-NHLs include Burkitt's lymphoma (BL), Burkitt-like lymphoma, small non-cleaved cell lymphoma (SNCCL) and lymphoblastic lymphoma. Other B-NHLs include immunoblastic lymphoma (or immunocytoma), primary effusion lymphoma, HIV associated (or AIDS related) lymphomas, and post-transplant lymphoproliferative disorder (PTLD) or lymphoma. B-cell malignancies also include, but are not limited to, chronic lymphocytic leukemia (CLL), prolymphocytic leukemia (PLL), Waldenstrom's macroglobulinemia (WM), hairy cell leukemia (HCL), large granular lymphocyte (LGL) leukemia, acute lymphoid (or lymphocytic or lymphoblastic) leukemia, and Castleman's disease. NHL may also include T-cell non-Hodgkin's lymphoma s(T-NHLs), which include, but are not limited to T-cell non-Hodgkin's lymphoma not otherwise specified (NOS), peripheral T-cell lymphoma (PTCL), anaplastic large cell lymphoma (ALCL), angioimmunoblastic lymphoid disorder (AILD), nasal natural killer (NK) cell/T-cell lymphoma, gamma/delta lymphoma, cutaneous T cell lymphoma, mycosis fungoides, and Sezary syndrome, among others.

Hematopoietic cancers also include Hodgkin's lymphoma (or disease) including classical Hodgkin's lymphoma, nodular sclerosing Hodgkin's lymphoma, mixed cellularity Hodgkin's lymphoma, lymphocyte predominant (LP) Hodgkin's lymphoma, nodular LP Hodgkin's lymphoma, and lymphocyte depleted Hodgkin's lymphoma. Hematopoietic cancers also include plasma cell diseases or cancers such as multiple myeloma (MM) including smoldering MM, monoclonal gammopathy of undetermined (or unknown or unclear) significance (MGUS), plasmacytoma (bone, extramedullary), lymphoplasmacytic lymphoma (LPL), Waldenstroem's Macroglobulinemia, plasma cell leukemia, and primary amyloidosis (AL). Hematopoietic cancers may also include other cancers of additional hematopoietic cells, including polymorphonuclear leukocytes (or neutrophils), basophils, eosinophils, dendritic cells, platelets, erythrocytes and natural killer cells. Tissues which include hematopoietic cells referred herein to as “hematopoietic cell tissues” include bone marrow; peripheral blood; thymus; and peripheral lymphoid tissues, such as spleen, lymph nodes, lymphoid tissues associated with mucosa (such as the gut-associated lymphoid tissues), tonsils, Peyer's patches and appendix, and lymphoid tissues associated with other mucosa, for example, the bronchial linings.

In one embodiment, the cancer is selected from the group consisting of colorectal cancer (CRC), gastric, esophageal, cervical, bladder, breast, head and neck, ovarian, melanoma, renal cell carcinoma (RCC), EC squamous cell, non-small cell lung carcinoma, mesothelioma, pancreatic, and prostate cancer.

The term “treating” and derivatives thereof as used herein, is meant to include therapeutic therapy. In reference to a particular condition, treating means: (1) to ameliorate the condition or one or more of the biological manifestations of the condition; (2) to interfere with (a) one or more points in the biological cascade that leads to or is responsible for the condition or (b) one or more of the biological manifestations of the condition; (3) to alleviate one or more of the symptoms, effects or side effects associated with the condition or one or more of the symptoms, effects or side effects associated with the condition or treatment thereof; (4) to slow the progression of the condition or one or more of the biological manifestations of the condition and/or (5) to cure said condition or one or more of the biological manifestations of the condition by eliminating or reducing to undetectable levels one or more of the biological manifestations of the condition for a period of time considered to be a state of remission for that manifestation without additional treatment over the period of remission. One skilled in the art will understand the duration of time considered to be remission for a particular disease or condition.

B-cell disorders can be divided into defects of B-cell development/immunoglobulin production (e.g. immunodeficiencies) and excessive/uncontrolled proliferation (e.g. lymphomas, leukemias). As used herein, B-cell disorder refers to both types of diseases, and methods are provided for treating B-cell disorders with the compositions described herein.

In a particular aspect, the disease or disorder is Multiple Myeloma (MM), Chronic Lymphocytic Leukaemia (CLL), Solitary Plasmacytoma (Bone, Extramedullary), amyloidosis (AL), Smoldering Multiple Myeloma (SMM), Solitary Plasmacytoma (Bone, Extramedullary), or Waldenstrom's Macroglobulinemia.

Prophylactic therapy is also contemplated. The skilled artisan will appreciate that “prevention” is not an absolute term. In medicine, “prevention” is understood to refer to the prophylactic administration of a drug to substantially diminish the likelihood or severity of a condition or biological manifestation thereof, or to delay the onset of such condition or biological manifestation thereof. Prophylactic therapy is appropriate, for example, when a subject is considered at high risk for developing cancer, such as when a subject has a strong family history of cancer or when a subject has been exposed to a carcinogen.

“Subject” or “patient” are used interchangeably herein and are defined broadly to include any person in need of treatment, for example, a person in need of cancer treatment. A subject may include a mammal. In one embodiment, the subject is a human patient. The subject in need of cancer treatment may include patients from a variety of stages including newly diagnosed, relapsed, refractory, progressive disease, remission, and others. The subject in need of cancer treatment may also include patients who have undergone stem cell transplant or who are considered transplant ineligible.

Subjects may be pre-screened in order to be selected for treatment with the compositions described herein. In one embodiment, a sample from the subject is tested for expression of BCMA prior to treatment with the compositions described herein.

Subjects may have had at least one prior cancer therapy before being treated with the compositions of the present invention. In one embodiment, the subject has been treated with at least 1, at least 2, at least 3, at least 4, at least 5, at least 6, or at least 7 prior cancer therapies before being treated with the compositions of the present invention.

In another embodiment, the subject has newly diagnosed cancer and has had 0 prior therapies before being treated with the compositions of the present invention.

The compositions of the invention may be administered by any appropriate route. For some compositions, suitable routes include oral, rectal, nasal, topical (including buccal and sublingual), vaginal, parenteral (including subcutaneous, intramuscular, intravenous, intradermal, intrathecal, and epidural), and intratumorally. It will be appreciated that the preferred route may vary with, for example, the condition of the recipient and the cancer to be treated.

In certain embodiments, a composition of the invention are administered as a pharmaceutical composition.

The term “administering” as used herein is meant to refer to the delivery of the compositions described herein to achieve a therapeutic objective. The compositions may be administered at an administration interval for a period sufficient to achieve clinical benefit. The composition may be administered to the subject in such a way as to target therapy to a particular site.

In some embodiments, the composition is administered by injection. Therefore, in one aspect there is provided an injection device comprising the composition, pharmaceutical composition or formulation of the invention. The injection device may comprise a pen injector device or an autoinjector device.

The term “therapeutically effective amount” or “therapeutically effective dose” of a composition as used herein refers to an amount effective in the prevention or treatment or alleviation of a symptom of a B-cell mediated disorder or disorder. Therapeutically effective amounts and treatment regimes are generally determined empirically and may be dependent on factors, such as the age, weight, and health status of the patient and disease or disorder to be treated. Such factors are within the purview of the attending physician.

The appropriate therapeutically effective dose of the composition comprising an anti-BCMA antigen binding protein will be determined readily by those of skill in the art. Suitable doses of the compositions described herein may be calculated for patients according to their weight, for example suitable doses may be in the range of about 0.1 mg/kg to about 20 mg/kg, for example about 1 mg/kg to about 20 mg/kg, for example about 10 mg/kg to about 20 mg/kg or for example about 1 mg/kg to about 15 mg/kg, for example about 10 mg/kg to about 15 mg/kg.

In one embodiment, a therapeutically effective dose of the composition comprising an anti-BCMA antigen binding protein is in the range of about 0.03 mg/kg to about 4.6 mg/kg. In yet another embodiment, a therapeutically effective dose of the composition comprising an anti-BCMA antigen binding protein is 0.03 mg/kg, 0.06 mg/kg, 0.12 mg/kg, 0.24 mg/kg, 0.48 mg/kg, 0.96 mg/kg, 1.92 mg/kg, 3.4 mg/kg, or 4.6 mg/kg. In yet another embodiment, a therapeutically effective dose of the composition comprising an anti-BCMA antigen binding protein is 1.9 mg/kg, 2.5 mg/kg or 3.4 mg/kg.

In certain embodiments, a composition can be co-administered to a subject with one or more additional therapeutic agents. In another embodiment, a composition can be co-administered to a subject with one or more additional cancer therapeutics. The additional cancer therapeutic agent may include, but is not limited to, other immunomodulatory drugs, therapeutic antibodies (e.g., an anti-CD38 antibody such as daratumumab), CAR-T therapeutics, BiTEs, HDAC inhibitors, proteasome inhibitors (e.g., bortezomib), anti-inflammatory compounds, and immunomodulatory imide drugs (IMiD) (e.g., thalidomide and analogs thereof).

“Co-administered” means the administration of two or more different pharmaceutical compositions or treatments (e.g., radiation treatment) that are administered to a subject by combination in the same pharmaceutical composition or separate pharmaceutical compositions. Thus, co-administration involves administration at the same time of a single pharmaceutical composition comprising two or more pharmaceutical agents or administration of two or more different compositions to the same subject at the same or different times.

In one aspect of the invention, the invention provides a method of treating a B-cell disease or disorder in a subject in need thereof by administering a therapeutically effective dose of any of the compositions comprising an anti-BCMA antigen binding protein as described herein.

In one embodiment, the invention provides a method of treating cancer in a subject in need thereof comprising administering a therapeutically effective dose of a composition comprising an anti-BCMA ADC, wherein the average DAR is about 3.4 to about 4.6.

In another embodiment, the invention provides a method of treating cancer in a subject in need thereof comprising administering a therapeutically effective dose of a composition comprising an anti-BCMA ADC, wherein the antibody comprises a CDRH1 with the amino acid sequence set forth in SEQ ID NO:1, a CDRH2 with the amino acid sequence set forth in SEQ ID NO:2, a CDRH3 with the amino acid sequence set forth in SEQ ID NO:3, a CDRL1 with the amino acid sequence set forth in SEQ ID NO:4, a CDRL2 with the amino acid sequence set forth in SEQ ID NO:5, and a CDRL3 with the amino acid sequence set forth in SEQ ID NO:6; wherein the cytotoxic agent is MMAE or MMAF; and wherein the average DAR is about 3.4 to about 4.6.

In another embodiment, the invention provides a method of treating cancer in a subject in need thereof comprising administering a therapeutically effective dose of a composition comprising an anti-BCMA ADC, wherein the antibody comprises a V_{H} with the amino acid sequence set forth in SEQ ID NO:7, and a V_{L} with the amino acid sequence set forth in SEQ ID NO:8; wherein the cytotoxic agent is MMAE or MMAF; and wherein the average DAR is about 3.4 to about 4.6.

In yet another embodiment, the invention provides a method of treating cancer in a subject in need thereof comprising administering a therapeutically effective dose of a composition comprising belantamab mafodotin, wherein the average DAR is 3.4 to about 4.6.

In yet another embodiment, the invention provides a method of treating multiple myeloma in a subject in need thereof comprising administering a therapeutically effective dose of a composition comprising belantamab mafodotin, wherein the average DAR is 3.4 to about 4.6.

In one embodiment, the invention provides a method of treating cancer in a subject in need thereof comprising administering a therapeutically effective dose of a composition comprising an anti-BCMA ADC; wherein percent DL0 is less than or equal to about 10% or about 5%, percent DL2 is about 15% to about 27% or about 15% to about 32%, percent DL4a is about 35% to about 38% or about 30% to about 40%, percent DL4b is about 7% to about 9% or about 5% to about 10%, percent DL6 is about 14% to about 20% or about 10% to about 20%, and/or DL8 is about 6.0% to about 12.0% or about 4% to about 15%.

In another embodiment, the invention provides a method of treating cancer in a subject in need thereof comprising administering a therapeutically effective dose of a composition comprising an anti-BCMA ADC, wherein the antibody comprises a CDRH1 with the amino acid sequence set forth in SEQ ID NO:1, a CDRH2 with the amino acid sequence set forth in SEQ ID NO:2, a CDRH3 with the amino acid sequence set forth in SEQ ID NO:3, a CDRL1 with the amino acid sequence set forth in SEQ ID NO:4, a CDRL2 with the amino acid sequence set forth in SEQ ID NO:5, and a CDRL3 with the amino acid sequence set forth in SEQ ID NO:6; wherein the cytotoxic agent is MMAE or MMAF; and wherein percent DL0 is less than or equal to about 10% or about 5%, percent DL2 is about 15% to about 27% or about 15% to about 32%, percent DL4a is about 35% to about 38% or about 30% to about 40%, percent DL4b is about 7% to about 9% or about 5% to about 10%, percent DL6 is about 14% to about 20% or about 10% to about 20%, and/or DL8 is about 6.0% to about 12.0% or about 4% to about 15%.

In another embodiment, the invention provides a method of treating cancer in a subject in need thereof comprising administering a therapeutically effective dose of a composition comprising an anti-BCMA ADC, wherein the antibody comprises a V_{H} with the amino acid sequence set forth in SEQ ID NO:7, and a V_{L} with the amino acid sequence set forth in SEQ ID NO:8; wherein the cytotoxic agent is MMAE or MMAF; and wherein percent DL0 is less than or equal to about 10% or about 5%, percent DL2 is about 15% to about 27% or about 15% to about 32%, percent DL4a is about 35% to about 38% or about 30% to about 40%, percent DL4b is about 7% to about 9% or about 5% to about 10%, percent DL6 is about 14% to about 20% or about 10% to about 20%, and/or DL8 is about 6.0% to about 12.0% or about 4% to about 15%.

In yet another embodiment, the invention provides a method of treating cancer in a subject in need thereof comprising administering a therapeutically effective dose of a composition comprising belantamab mafodotin, wherein percent DL0 is less than or equal to about 10% or about 5%, percent DL2 is about 15% to about 27% or about 15% to about 32%, percent DL4a is about 35% to about 38% or about 30% to about 40%, percent DL4b is about 7% to about 9% or about 5% to about 10%, percent DL6 is about 14% to about 20% or about 10% to about 20%, and/or DL8 is about 6.0% to about 12.0% or about 4% to about 15%.

In yet another embodiment, the invention provides a method of treating multiple myeloma in a subject in need thereof comprising administering a therapeutically effective dose of a composition comprising belantamab mafodotin, wherein percent DL0 is less than or equal to about 10% or about 5%, percent DL2 is about 15% to about 27% or about 15% to about 32%, percent DL4a is about 35% to about 38% or about 30% to about 40%, percent DL4b is about 7% to about 9% or about 5% to about 10%, percent DL6 is about 14% to about 20% or about 10% to about 20%, and/or DL8 is about 6.0% to about 12.0% or about 4% to about 15%.

In one aspect, the invention provides a method of treating cancer in a subject in need thereof comprising administering a therapeutically effective dose of a composition comprising an antibody comprising a CDRH1 with the amino acid sequence set forth in SEQ ID NO:1, a CDRH2 with the amino acid sequence set forth in SEQ ID NO:2, a CDRH3 with the amino acid sequence set forth in SEQ ID NO:3, a CDRL1 with the amino acid sequence set forth in SEQ ID NO:4, a CDRL2 with the amino acid sequence set forth in SEQ ID NO:5, and a CDRL3 with the amino acid sequence set forth in SEQ ID NO:6; wherein the composition comprises ≤25% isomerization at heavy chain D103 at CDRH3.

In one embodiment, the invention provides a method of treating multiple myeloma in a subject in need thereof comprising administering a therapeutically effective dose of a composition comprising an antibody comprising a CDRH1 with the amino acid sequence set forth in SEQ ID NO:1, a CDRH2 with the amino acid sequence set forth in SEQ ID NO:2, a CDRH3 with the amino acid sequence set forth in SEQ ID NO:3, a CDRL1 with the amino acid sequence set forth in SEQ ID NO:4, a CDRL2 with the amino acid sequence set forth in SEQ ID NO:5, and a CDRL3 with the amino acid sequence set forth in SEQ ID NO:6; wherein the composition comprises ≤25% isomerization at heavy chain D103 at CDRH3.

In one embodiment, the invention provides a method of treating multiple myeloma in a subject in need thereof comprising administering a therapeutically effective dose of a composition comprising belantamab; wherein the composition comprises ≤25% isomerization at heavy chain D103 at CDRH3.

In one aspect, the invention provides a method of treating cancer in a subject in need thereof comprising administering a therapeutically effective dose of a composition comprising an antibody comprising a CDRH1 with the amino acid sequence set forth in SEQ ID NO:1, a CDRH2 with the amino acid sequence set forth in SEQ ID NO:2, a CDRH3 with the amino acid sequence set forth in SEQ ID NO:3, a CDRL1 with the amino acid sequence set forth in SEQ ID NO:4, a CDRL2 with the amino acid sequence set forth in SEQ ID NO:5, and a CDRL3 with the amino acid sequence set forth in SEQ ID NO:6; wherein the composition comprises ≤40% oxidation at heavy chain M34 (CDRH1).

In embodiment, the invention provides a method of treating multiple myeloma in a subject in need thereof comprising administering a therapeutically effective dose of a composition comprising an antibody comprising a CDRH1 with the amino acid sequence set forth in SEQ ID NO:1, a CDRH2 with the amino acid sequence set forth in SEQ ID NO:2, a CDRH3 with the amino acid sequence set forth in SEQ ID NO:3, a CDRL1 with the amino acid sequence set forth in SEQ ID NO:4, a CDRL2 with the amino acid sequence set forth in SEQ ID NO:5, and a CDRL3 with the amino acid sequence set forth in SEQ ID NO:6; wherein the composition comprises ≤40% oxidation at heavy chain M34 (CDRH1).

In embodiment, the invention provides a method of treating multiple myeloma in a subject in need thereof comprising administering a therapeutically effective dose of a composition comprising belantamab; wherein the composition comprises ≤40% oxidation at heavy chain M34 (CDRH1).

In one aspect of the invention, the invention provides a composition comprising an anti-BCMA antigen binding protein, as described herein, for use in the treatment of B-cell disease or disorder.

In one embodiment, the invention provides a composition comprising an anti-BCMA ADC, as described herein, for the use in the treatment of cancer, wherein the average DAR is about 3.4 to about 4.6.

In another embodiment, the invention provides a composition comprising an anti-BCMA ADC, as described herein, for the use in the treatment of cancer, wherein the antibody comprises a CDRH1 with the amino acid sequence set forth in SEQ ID NO:1, a CDRH2 with the amino acid sequence set forth in SEQ ID NO:2, a CDRH3 with the amino acid sequence set forth in SEQ ID NO:3, a CDRL1 with the amino acid sequence set forth in SEQ ID NO:4, a CDRL2 with the amino acid sequence set forth in SEQ ID NO:5, and a CDRL3 with the amino acid sequence set forth in SEQ ID NO:6; wherein the cytotoxic agent is MMAE or MMAF; and wherein the average DAR is about 3.4 to about 4.6.

In another embodiment, the invention provides a composition comprising an anti-BCMA ADC, as described herein, for the use in the treatment of cancer, wherein the antibody comprises a V_{H} with the amino acid sequence set forth in SEQ ID NO:7, and a V_{L} with the amino acid sequence set forth in SEQ ID NO:8; wherein the cytotoxic agent is MMAE or MMAF; and wherein the average DAR is about 3.4 to about 4.6.

In another embodiment, the invention provides a composition comprising belantamab mafodotin for the use in the treatment of cancer, wherein the average DAR is 3.4 to about 4.6.

In yet another embodiment, the invention provides a composition comprising belantamab mafodotin for the use in the treatment of multiple myeloma, wherein the average DAR is 3.4 to about 4.6.

In one embodiment, the invention provides a composition comprising an anti-BCMA ADC, as described herein, for the use in the treatment of cancer; wherein percent DL0 is less than or equal to about 10% or about 5%, percent DL2 is about 15% to about 27% or about 15% to about 32%, percent DL4a is about 35% to about 38% or about 30% to about 40%, percent DL4b is about 7% to about 9% or about 5% to about 10%, percent DL6 is about 14% to about 20% or about 10% to about 20%, and/or DL8 is about 6.0% to about 12.0% or about 4% to about 15%.

In another embodiment, the invention provides a composition comprising an anti-BCMA ADC, as described herein, for the use in the treatment of cancer, wherein the antibody comprises a CDRH1 with the amino acid sequence set forth in SEQ ID NO:1, a CDRH2 with the amino acid sequence set forth in SEQ ID NO:2, a CDRH3 with the amino acid sequence set forth in SEQ ID NO:3, a CDRL1 with the amino acid sequence set forth in SEQ ID NO:4, a CDRL2 with the amino acid sequence set forth in SEQ ID NO:5, and a CDRL3 with the amino acid sequence set forth in SEQ ID NO:6; wherein the cytotoxic agent is MMAE or MMAF; and wherein percent DL0 is less than or equal to about 10% or about 5%, percent DL2 is about 15% to about 27% or about 15% to about 32%, percent DL4a is about 35% to about 38% or about 30% to about 40%, percent DL4b is about 7% to about 9% or about 5% to about 10%, percent DL6 is about 14% to about 20% or about 10% to about 20%, and/or DL8 is about 6.0% to about 12.0% or about 4% to about 15%.

In another embodiment, the invention provides a composition comprising an anti-BCMA ADC, as described herein, for the use in the treatment of cancer, wherein the antibody comprises a V_{H} with the amino acid sequence set forth in SEQ ID NO:7, and a V_{L} with the amino acid sequence set forth in SEQ ID NO:8; wherein the cytotoxic agent is MMAE or MMAF; and wherein percent DL0 is less than or equal to about 10% or about 5%, percent DL2 is about 15% to about 27% or about 15% to about 32%, percent DL4a is about 35% to about 38% or about 30% to about 40%, percent DL4b is about 7% to about 9% or about 5% to about 10%, percent DL6 is about 14% to about 20% or about 10% to about 20%, and/or DL8 is about 6.0% to about 12.0% or about 4% to about 15%.

In another embodiment, the invention provides a composition comprising belantamab mafodotin for the use in the treatment of cancer, wherein percent DL0 is less than or equal to about 10% or about 5%, percent DL2 is about 15% to about 26%, percent DL4a is about 35% to about 38%, DL4b is about 7% to about 10%, percent DL6 is about 14% to about 20%, and/or percent DL8 is about 6% to about 12%.

In yet another embodiment, the invention provides a composition comprising belantamab mafodotin for the use in the treatment of multiple myeloma, wherein percent DL0 is less than or equal to about 10% or about 5%, percent DL2 is about 15% to about 27% or about 15% to about 32%, percent DL4a is about 35% to about 38% or about 30% to about 40%, percent DL4b is about 7% to about 9% or about 5% to about 10%, percent DL6 is about 14% to about 20% or about 10% to about 20%, and/or DL8 is about 6.0% to about 12.0% or about 4% to about 15%.

In one aspect, the invention provides a composition comprising an antibody for the use in the treatment of cancer, wherein the antibody comprises a CDRH1 with the amino acid sequence set forth in SEQ ID NO:1, a CDRH2 with the amino acid sequence set forth in SEQ ID NO:2, a CDRH3 with the amino acid sequence set forth in SEQ ID NO:3, a CDRL1 with the amino acid sequence set forth in SEQ ID NO:4, a CDRL2 with the amino acid sequence set forth in SEQ ID NO:5, and a CDRL3 with the amino acid sequence set forth in SEQ ID NO:6; and wherein the composition comprises ≤25% isomerization at heavy chain D103 at CDRH3.

In one aspect, the invention provides a composition comprising an antibody for the use in the treatment of multiple myeloma, wherein the antibody comprises a CDRH1 with the amino acid sequence set forth in SEQ ID NO:1, a CDRH2 with the amino acid sequence set forth in SEQ ID NO:2, a CDRH3 with the amino acid sequence set forth in SEQ ID NO:3, a CDRL1 with the amino acid sequence set forth in SEQ ID NO:4, a CDRL2 with the amino acid sequence set forth in SEQ ID NO:5, and a CDRL3 with the amino acid sequence set forth in SEQ ID NO:6; and wherein the composition comprises ≤25% isomerization at heavy chain D103 at CDRH3.

In yet another embodiment, the invention provides a composition comprising belantamab mafodotin for the use in the treatment of multiple myeloma, wherein the composition comprises ≤25% isomerization at heavy chain D103 at CDRH3.

In one aspect, the invention provides a composition comprising an antibody for the use in the treatment of cancer, wherein the antibody comprises a CDRH1 with the amino acid sequence set forth in SEQ ID NO:1, a CDRH2 with the amino acid sequence set forth in SEQ ID NO:2, a CDRH3 with the amino acid sequence set forth in SEQ ID NO:3, a CDRL1 with the amino acid sequence set forth in SEQ ID NO:4, a CDRL2 with the amino acid sequence set forth in SEQ ID NO:5, and a CDRL3 with the amino acid sequence set forth in SEQ ID NO:6; and wherein the composition comprises ≤40% oxidation at heavy chain M34 (CDRH1).

In one aspect, the invention provides a composition comprising an antibody for the use in the treatment of multiple myeloma, wherein the antibody comprises a CDRH1 with the amino acid sequence set forth in SEQ ID NO:1, a CDRH2 with the amino acid sequence set forth in SEQ ID NO:2, a CDRH3 with the amino acid sequence set forth in SEQ ID NO:3, a CDRL1 with the amino acid sequence set forth in SEQ ID NO:4, a CDRL2 with the amino acid sequence set forth in SEQ ID NO:5, and a CDRL3 with the amino acid sequence set forth in SEQ ID NO:6; and wherein the composition comprises ≤40% oxidation at heavy chain M34 (CDRH1).

In yet another embodiment, the invention provides a composition comprising belantamab mafodotin for the use in the treatment of multiple myeloma, wherein the composition comprises ≤40% oxidation at heavy chain M34 (CDRH1).

In one aspect of the invention, provided is the use of a composition in the manufacture of a medicament for use in the treatment of B-cell diseases or disorders.

In one embodiment, provided is the use of a composition comprising an anti-BCMA ADC in the manufacture of a medicament for use in the treatment of cancer, wherein the average DAR is about 3.4 to about 4.6.

In another embodiment, provided is the use of a composition comprising an anti-BCMA ADC in the manufacture of a medicament for use in the treatment of cancer, wherein the antibody comprises a CDRH1 with the amino acid sequence set forth in SEQ ID NO:1, a CDRH2 with the amino acid sequence set forth in SEQ ID NO:2, a CDRH3 with the amino acid sequence set forth in SEQ ID NO:3, a CDRL1 with the amino acid sequence set forth in SEQ ID NO:4, a CDRL2 with the amino acid sequence set forth in SEQ ID NO:5, and a CDRL3 with the amino acid sequence set forth in SEQ ID NO:6; wherein the cytotoxic agent is MMAE or MMAF; and wherein the average DAR is about 3.4 to about 4.6.

In another embodiment, provided is the use of a composition comprising an anti-BCMA ADC in the manufacture of a medicament for use in the treatment of cancer, wherein the antibody comprises a V_{H} with the amino acid sequence set forth in SEQ ID NO:7, and a V_{L} with the amino acid sequence set forth in SEQ ID NO:8; wherein the cytotoxic agent is MMAE or MMAF; and wherein the average DAR is about 3.4 to about 4.6.

In another embodiment, provided is the use of a composition comprising belantamab mafodotin in the manufacture of a medicament for use in the treatment of cancer, wherein the average DAR is 3.4 to about 4.6.

In another embodiment, provided is the use of a composition comprising belantamab mafodotin in the manufacture of a medicament for use in the treatment of multiple myeloma, wherein the average DAR is 3.4 to about 4.6.

In one embodiment, provided is the use of a composition comprising an anti-BCMA ADC in the manufacture of a medicament for use in the treatment of cancer; wherein percent DL0 is less than or equal to about 10% or about 5%, percent DL2 is about 15% to about 27% or about 15% to about 32%, percent DL4a is about 35% to about 38% or about 30% to about 40%, percent DL4b is about 7% to about 9% or about 5% to about 10%, percent DL6 is about 14% to about 20% or about 10% to about 20%, and/or DL8 is about 6.0% to about 12.0% or about 4% to about 15%.

In another embodiment, provided is the use of a composition comprising an anti-BCMA ADC in the manufacture of a medicament for use in the treatment of cancer, wherein the antibody comprises a CDRH1 with the amino acid sequence set forth in SEQ ID NO:1, a CDRH2 with the amino acid sequence set forth in SEQ ID NO:2, a CDRH3 with the amino acid sequence set forth in SEQ ID NO:3, a CDRL1 with the amino acid sequence set forth in SEQ ID NO:4, a CDRL2 with the amino acid sequence set forth in SEQ ID NO:5, and a CDRL3 with the amino acid sequence set forth in SEQ ID NO:6; wherein the cytotoxic agent is MMAE or MMAF; and wherein percent DL0 is less than or equal to about 10% or about 5%, percent DL2 is about 15% to about 27% or about 15% to about 32%, percent DL4a is about 35% to about 38% or about 30% to about 40%, percent DL4b is about 7% to about 9% or about 5% to about 10%, percent DL6 is about 14% to about 20% or about 10% to about 20%, and/or DL8 is about 6.0% to about 12.0% or about 4% to about 15%.

In another embodiment, provided is the use of a composition comprising an anti-BCMA ADC in the manufacture of a medicament for use in the treatment of cancer, wherein the antibody comprises a V_{H} with the amino acid sequence set forth in SEQ ID NO:7, and a V_{L} with the amino acid sequence set forth in SEQ ID NO:8; wherein the cytotoxic agent is MMAE or MMAF; and wherein percent DL0 is less than or equal to about 10% or about 5%, percent DL2 is about 15% to about 27% or about 15% to about 32%, percent DL4a is about 35% to about 38% or about 30% to about 40%, percent DL4b is about 7% to about 9% or about 5% to about 10%, percent DL6 is about 14% to about 20% or about 10% to about 20%, and/or DL8 is about 6.0% to about 12.0% or about 4% to about 15%.

In another embodiment, provided is the use of a composition comprising an anti-BCMA ADC in the manufacture of a medicament for use in the treatment of cancer, wherein percent DL0 is less than or equal to about 10% or about 5%, percent DL2 is about 15% to about 27% or about 15% to about 32%, percent DL4a is about 35% to about 38% or about 30% to about 40%, percent DL4b is about 7% to about 9% or about 5% to about 10%, percent DL6 is about 14% to about 20% or about 10% to about 20%, and/or DL8 is about 6.0% to about 12.0% or about 4% to about 15%.

In another embodiment, provided is the use of a composition comprising belantamab mafodotin in the manufacture of a medicament for use in the treatment of cancer, wherein percent DL0 is less than or equal to about 10% or about 5%, percent DL2 is about 15% to about 27% or about 15% to about 32%, percent DL4a is about 35% to about 38% or about 30% to about 40%, percent DL4b is about 7% to about 9% or about 5% to about 10%, percent DL6 is about 14% to about 20% or about 10% to about 20%, and/or DL8 is about 6.0% to about 12.0% or about 4% to about 15%.

In yet another embodiment, provided is the use of a composition comprising belantamab mafodotin in the manufacture of a medicament for use in the treatment of multiple myeloma, wherein percent DL0 is less than or equal to about 10% or about 5%, percent DL2 is about 15% to about 27% or about 15% to about 32%, percent DL4a is about 35% to about 38% or about 30% to about 40%, percent DL4b is about 7% to about 9% or about 5% to about 10%, percent DL6 is about 14% to about 20% or about 10% to about 20%, and/or DL8 is about 6.0% to about 12.0% or about 4% to about 15%.

In one aspect, provided is the use of a composition comprising an anti-BCMA antigen binding protein in the manufacture of a medicament for use in the treatment of cancer, wherein the composition comprising an antibody comprising a CDRH1 with the amino acid sequence set forth in SEQ ID NO:1, a CDRH2 with the amino acid sequence set forth in SEQ ID NO:2, a CDRH3 with the amino acid sequence set forth in SEQ ID NO:3, a CDRL1 with the amino acid sequence set forth in SEQ ID NO:4, a CDRL2 with the amino acid sequence set forth in SEQ ID NO:5, and a CDRL3 with the amino acid sequence set forth in SEQ ID NO:6; and wherein the composition comprises ≤25% isomerization at heavy chain D103 at CDRH3.

In one embodiment, provided is the use of a composition comprising an anti-BCMA antigen binding protein in the manufacture of a medicament for use in the multiple myeloma, wherein the composition comprising an antibody comprising a CDRH1 with the amino acid sequence set forth in SEQ ID NO:1, a CDRH2 with the amino acid sequence set forth in SEQ ID NO:2, a CDRH3 with the amino acid sequence set forth in SEQ ID NO:3, a CDRL1 with the amino acid sequence set forth in SEQ ID NO:4, a CDRL2 with the amino acid sequence set forth in SEQ ID NO:5, and a CDRL3 with the amino acid sequence set forth in SEQ ID NO:6; and wherein the composition comprises ≤25% isomerization at heavy chain D103 at CDRH3.

In yet another embodiment, provided is the use of a composition composing belantamab in the manufacture of a medicament for use in the treatment of multiple myeloma, wherein the composition comprises ≤25% isomerization at heavy chain D103 at CDRH3.

In one aspect, provided is the use of a composition comprising an anti-BCMA antigen binding protein in the manufacture of a medicament for use in the treatment of cancer, wherein the composition comprising an antibody comprising a CDRH1 with the amino acid sequence set forth in SEQ ID NO:1, a CDRH2 with the amino acid sequence set forth in SEQ ID NO:2, a CDRH3 with the amino acid sequence set forth in SEQ ID NO:3, a CDRL1 with the amino acid sequence set forth in SEQ ID NO:4, a CDRL2 with the amino acid sequence set forth in SEQ ID NO:5, and a CDRL3 with the amino acid sequence set forth in SEQ ID NO:6; and wherein the composition comprises ≤40%, oxidation at heavy chain M34 (CDRH1).

In one embodiment, provided is the use of a composition comprising an anti-BCMA antigen binding protein in the manufacture of a medicament for use in the multiple myeloma, wherein the composition comprising an antibody comprising a CDRH1 with the amino acid sequence set forth in SEQ ID NO:1, a CDRH2 with the amino acid sequence set forth in SEQ ID NO:2, a CDRH3 with the amino acid sequence set forth in SEQ ID NO:3, a CDRL1 with the amino acid sequence set forth in SEQ ID NO:4, a CDRL2 with the amino acid sequence set forth in SEQ ID NO:5, and a CDRL3 with the amino acid sequence set forth in SEQ ID NO:6; and wherein the composition comprises ≤40%, oxidation at heavy chain M34 (CDRH1).

In yet another embodiment, provided is the use of a composition composing belantamab in the manufacture of a medicament for use in the treatment of multiple myeloma, wherein the composition comprises ≤40%, oxidation at heavy chain M34 (CDRH1).

All patent and literature references disclosed herein are expressly and entirely incorporated herein by reference.

The invention described herein comprises:

1. A composition comprising an isomerized variant of an anti-BCMA antibody, wherein the isomerized variant comprises a heavy chain amino acid sequence comprising a CDRH1 of SEQ ID NO: 1, a CDRH2 of SEQ ID NO: 2, and a CDRH3 of SEQ ID NO: 3, and a light chain amino acid sequence comprising a CDRL1 of SEQ ID NO: 4, a CDRL2 of SEQ ID NO: 5, and a CDRL3 of SEQ ID NO: 6; wherein the composition comprises ≤25% isomerized variant.

2. A composition comprising an oxidized variant of an anti-BCMA antibody, wherein the oxidized variant comprises a heavy chain amino acid sequence comprising a CDRH1 of SEQ ID NO: 1, a CDRH2 of SEQ ID NO: 2, and a CDRH3 of SEQ ID NO: 3, and a light chain amino acid sequence comprising a CDRL1 of SEQ ID NO: 4, a CDRL2 of SEQ ID NO: 5, and a CDRL3 of SEQ ID NO: 6; wherein the composition comprises ≤40%, oxidized variant.

3. A composition comprising an anti-BCMA antibody comprising a CDRH1 with the amino acid sequence set forth in SEQ ID NO:1, a CDRH2 with the amino acid sequence set forth in SEQ ID NO:2, a CDRH3 with the amino acid sequence set forth in SEQ ID NO:3, a CDRL1 with the amino acid sequence set forth in SEQ ID NO:4, a CDRL2 with the amino acid sequence set forth in SEQ ID NO:5, and a CDRL3 with the amino acid sequence set forth in SEQ ID NO:6; wherein the composition comprises 0.1-25%, isomerization at D103 at CDRH3.

4. A composition comprising an anti-BCMA antibody comprising a CDRH1 with the amino acid sequence set forth in SEQ ID NO:1, a CDRH2 with the amino acid sequence set forth in SEQ ID NO:2, a CDRH3 with the amino acid sequence set forth in SEQ ID NO:3, a CDRL1 with the amino acid sequence set forth in SEQ ID NO:4, a CDRL2 with the amino acid sequence set forth in SEQ ID NO:5, and a CDRL3 with the amino acid sequence set forth in SEQ ID NO:6; wherein the composition comprises 0.1-40% oxidation at M34 at CDRH1.

5. A composition comprising an anti-BCMA antibody that is at least about 90% identical to the heavy chain amino acid sequence of SEQ ID NO:9 and the light chain amino acid sequence of SEQ ID NO:10, wherein the composition comprises 0.1-25%, isomerization at D103 at CDRH3.

6. A composition comprising an anti-BCMA antibody that is at least about 90% identical to the heavy chain amino acid sequence of SEQ ID NO:9 and the light chain amino acid sequence of SEQ ID NO:10, wherein the composition comprises 0.1-40% oxidation at M34 CDRH1.

7. The composition according to any preceding claim wherein the composition comprises ≤65%, oxidation at heavy chain M256 and/or ≤60%, oxidation at heavy chain M432.

8. The composition according to any preceding claim wherein the composition comprises an antibody variant comprising at least one selected from the group consisting of heavy chain deamidation at N388 and/or N393, D103 to N103 at CDRH3, C-terminal lysine cleavage, and N-terminal conversion of glutamine to pyroglutamic acid.

9. The composition according to any preceding claim wherein the composition comprises at least one selected from the group consisting of up to 100% deamidation at N388 and/or N393, up to 100% N103 at CDRH3, up to 100% C-terminal lysine cleavage, and up to 100% N-terminal conversion of glutamine to pyroglutamic acid.

10. The composition according to any preceding claim wherein the composition comprises any percentage of glycoforms G0, G1, G2, G0-GlcNac or G0-2GlcNac.

11. The composition according to any preceding claim wherein the anti-BCMA antibody is belantamab.

12. The composition according to any one of the preceding claims wherein the anti-BCMA antibody is conjugated to a cytotoxic agent to form an antibody-drug-conjugate.

13. The composition according to any one of the preceding claims wherein the anti-BCMA antibody is belantamab mafodotin.

14. The composition of claims 12-13, wherein the wherein percent DL2 is at least about 30%, about 15% to about 27%, or about 15% to about 32%; percent DL4a is at least about 30%, about 35% to about 38%, or about 30% to about 40%; percent DL4b is at least about 5%, about 7% to about 9%, or about 5% to about 10%; percent DL6 is at least about 10%, about 14% to about 20%, or about 10% to about 20%; and/or DL8 is at least about 1%, about 6.0% to about 12.0%, or about 4% to about 15%.

15. The composition of claims 12-14, wherein the average DAR is about 3.4 to about 4.6.

16. The composition of claims 12-15, wherein percent DL0 is less than or equal to about 10% or about 5%.

17. A pharmaceutical composition comprising the composition of any preceding claim and at least one pharmaceutically acceptable excipient.

18. A formulation comprising the pharmaceutical composition of claim 17 comprising an anti-BCMA antigen binding protein at about 20 mg/mL to about 60 mg/mL, citrate buffer at about 10 mM to about 30 mM, trehalose at about 120 mM to about 240 mM, EDTA at about 0.01 mM to about 0.1 mM, polysorbate 20 or polysorbate 80 at about 0.01% to about 0.05%, at a pH of about 5.9 to about 6.5.

19. The formulation of claim 18, comprising about 20 mg/mL, about 25 mg/mL, about 50 mg/mL, or about 60 mg/mL belantamab mafodotin, 25 mM citrate buffer, 200 mM trehalose, 0.05 mM disodium EDTA, 0.02% polysorbate 20 or polysorbate 80, at a pH of about 5.9 to about 6.5.

20. A method of treating cancer comprising administering to a subject in need thereof a therapeutically effective amount a composition of claims 1-16.

21. A composition of claims 1-14 for use in the treatment of cancer.

22. Use of a composition of claims 1-16 in the manufacture of a medicament for use in the treatment of cancer.

23. A composition comprising an anti-BCMA antibody-drug-conjugate (ADC), wherein the wherein percent DL2 is at least about 30%, about 15% to about 27%, or about 15% to about 32%; percent DL4a is at least about 30%, about 35% to about 38%, or about 30% to about 40%; percent DL4b is at least about 5%, about 7% to about 9%, or about 5% to about 10%; percent DL6 is at least about 10%, about 14% to about 20%, or about 10% to about 20%; and/or DL8 is at least about 1%, about 6.0% to about 12.0%, or about 4% to about 15%.

24. The composition of claim 23, wherein the antibody comprises a CDRH1 with the amino acid sequence set forth in SEQ ID NO:1, a CDRH2 with the amino acid sequence set forth in SEQ ID NO:2, a CDRH3 with the amino acid sequence set forth in SEQ ID NO:3, a CDRL1 with the amino acid sequence set forth in SEQ ID NO:4, a CDRL2 with the amino acid sequence set forth in SEQ ID NO:5, and a CDRL3 with the amino acid sequence set forth in SEQ ID NO:6.

25. The composition of claim 23 or 24, wherein percent DL2 is about 15% to about 32%, percent DL4a is about 30% to about 40%, percent DL4b is about 5% to about 10%, percent DL6 is about 10% to about 20%, and DL8 is about 4% to about 15%.

26. The composition of claim 23 or 24, wherein percent DL2 is about 15% to about 27%, percent DL4a is about 35% to about 38%, percent DL4b is about 7% to about 9%, percent DL6 is about 14% to about 20%, and DL8 is about 6.0% to about 12.0%.

27. The composition of claims 23-26, wherein the average drug-antibody ratio (DAR) is about 2.1 to about 5.7.

28. The composition of claims 23-26, wherein the average DAR is about 3.4 to about 4.6.

29. The composition of claims 23-26, wherein the average DAR is about 3.8 to about 4.5.

30. A composition comprising an anti-BCMA antibody-drug-conjugate (ADC), wherein percent DL0 is less than or equal to about 10% or about 5%.

31. The composition of claim 30, wherein the antibody comprises a CDRH1 with the amino acid sequence set forth in SEQ ID NO:1, a CDRH2 with the amino acid sequence set forth in SEQ ID NO:2, a CDRH3 with the amino acid sequence set forth in SEQ ID NO:3, a CDRL1 with the amino acid sequence set forth in SEQ ID NO:4, a CDRL2 with the amino acid sequence set forth in SEQ ID NO:5, and a CDRL3 with the amino acid sequence set forth in SEQ ID NO:6.

32. The composition of claim 30 or 31, wherein the percent DL0 is less than or equal to about 5%.

33. The composition of claims 30-32, wherein percent DL2 is about 15% to about 32%, percent DL4a is about 30% to about 40%, percent DL4b is about 5% to about 10%, percent DL6 is about 10% to about 20%, and DL8 is about 4% to about 15%.

34. The composition of claims 30-32, wherein percent DL2 is about 15% to about 27%, percent DL4a is about 35% to about 38%, percent DL4b is about 7% to about 9%, percent DL6 is about 14% to about 20%, and DL8 is about 6.0% to about 12.0%.

35. The composition of claims 30-34, wherein the average drug-antibody ratio (DAR) is about 2.1 to about 5.7.

36. The composition of claims 30-34, wherein the average DAR is about 3.4 to about 4.6.

37. The composition of claim 30-34, wherein the average DAR is about 3.8 to about 4.5.

38. The composition of claims 23-37, wherein the antibody comprises a V_{H} with the amino acid sequence set forth in SEQ ID NO:7, and a V_{L} with the amino acid sequence set forth in SEQ ID NO:8.

39. The composition of claims 23-38, wherein the antibody is belantamab.

40. The composition of claims 23-37, wherein the cytotoxic agent is MMAE or MMAF.

41. The composition of claims 21-40, wherein the anti-BCMA ADC is belantamab mafodotin.

42. The composition of claims 23-41, wherein the percent DL is determined by separating individual DL species using hydrophobic interaction chromatography (HIC), calculating the area under the curve for each DL peak, and dividing each DL peak by the total area under the curve for all DL species combined.

43. The composition of claim 42, wherein the average DAR is calculated from the area under the curve of each DL species using the following formula:

$$\% DL {Component}_{X} = \frac{{A}_{X}}{{A}_{0} + {A}_{1} + {A}_{2} + {A}_{3} + {A}_{4 a} + {A}_{4 b} + {A}_{5} + {A}_{6} + {A}_{8} + {A}_{10}} \times 100$$

$$DAR = \frac{\begin{matrix}( {A}_{1} \times 2 ) + ( {A}_{2} \times 2 ) + ( {A}_{3} \times 2 ) + ( {A}_{4 a} \times 4 ) + \\ ( {A}_{4 b} \times 4 ) + ( {A}_{5} \times 6 ) + ( {A}_{6} \times 6 ) + ( {A}_{8} \times 8 ) \times ( {A}_{10} \times 8 )\end{matrix}}{{A}_{0} + {A}_{1} + {A}_{2} + {A}_{3} + {A}_{4 a} + {A}_{4 b} + {A}_{5} + {A}_{6} + {A}_{8} + {A}_{10}}$$

$$Where : \\ {A}_{X} = Peak area of loading X peak \\ X = {A}_{0} , {A}_{1} , {A}_{2} , {A}_{3} , {A}_{4 a} , {A}_{4 b} , {A}_{5} , {A}_{6} , {A}_{8} , and {A}_{10}$$

$${A}_{0} , {A}_{1} , {A}_{2} , {A}_{3} , {A}_{4 a} , {A}_{4 b} , {A}_{5} , {A}_{6} , {A}_{8} , and {A}_{10} = is the peak of DL 0 , DL 1 , DL 2 , DL 3 ,$$

$$DL 4 a , DL 4 b , DL 5 , DL 6 , DL 8 and DL 10 peaks$$

$$( only including peaks \geq DL ( 0.08 \% ) )$$

44. A pharmaceutical composition comprising the composition of claims 23-43 and at least one pharmaceutically acceptable excipient.

45. A formulation comprising the pharmaceutical composition of claim 44 comprising an anti-BCMA antigen binding protein at about 20 mg/mL to about 60 mg/mL, citrate buffer at about 10 mM to about 30 mM, trehalose at about 120 mM to about 240 mM, EDTA at about 0.01 mM to about 0.1 mM, polysorbate 20 or polysorbate 80 at about 0.01% to about 0.05%, at a pH of about 5.9 to about 6.5.

46. The formulation of claim 45, comprising about 20 mg/mL, about 25 mg/mL, about 50 mg/mL, or about 60 mg/mL belantamab mafodotin, 25 mM citrate buffer, 200 mM trehalose, 0.05 mM disodium EDTA, 0.02% polysorbate 20 or polysorbate 80, at a pH of about 5.9 to about 6.5.

47. A method of treating cancer comprising administering to a subject in need thereof a therapeutically effective amount a composition of claims 23-43.

48. A composition of claims 23-43 for use in the treatment of cancer.

49. Use of a composition of claims 23-43 in the manufacture of a medicament for use in the treatment of cancer.

50. A composition comprising an acidic variant of an antibody, wherein the acidic variant comprises a CDRH1 of SEQ ID NO: 1, a CDRH2 of SEQ ID NO: 2, and a CDRH3 of SEQ ID NO: 3, and a light chain amino acid sequence comprising a CDRL1 of SEQ ID NO: 4, a CDRL2 of SEQ ID NO: 5, and a CDRL3 of SEQ ID NO: 6; wherein the composition comprises 1-70% acidic variant.

51. A composition comprising an acidic variant of an antibody, wherein the acidic variant comprises a CDRH1 of SEQ ID NO: 1, a CDRH2 of SEQ ID NO: 2, and a CDRH3 of SEQ ID NO: 3, and a light chain amino acid sequence comprising a CDRL1 of SEQ ID NO: 4, a CDRL2 of SEQ ID NO: 5, and a CDRL3 of SEQ ID NO: 6; wherein the composition comprises ≤70%, acidic variant.

52. A composition comprising a basic variant of an antibody, wherein the basic variant comprises a heavy chain amino acid sequence comprising a CDRH1 of SEQ ID NO: 1, a CDRH2 of SEQ ID NO: 2, and a CDRH3 of SEQ ID NO: 3, and a light chain amino acid sequence comprising a CDRL1 of SEQ ID NO: 4, a CDRL2 of SEQ ID NO: 5, and a CDRL3 of SEQ ID NO: 6; wherein the composition comprises 1-30% basic variant.

53. A composition comprising a basic variant of an antibody, wherein the basic variant comprises a heavy chain amino acid sequence comprising a CDRH1 of SEQ ID NO: 1, a CDRH2 of SEQ ID NO: 2, and a CDRH3 of SEQ ID NO: 3, and a light chain amino acid sequence comprising a CDRL1 of SEQ ID NO: 4, a CDRL2 of SEQ ID NO: 5, and a CDRL3 of SEQ ID NO: 6; wherein the composition comprises ≤30%, basic variant.

54. A composition comprising a main isoform of an antibody, wherein the main isoform comprises a heavy chain amino acid sequence comprising a CDRH1 of SEQ ID NO: 1, a CDRH2 of SEQ ID NO: 2, and a CDRH3 of SEQ ID NO: 3, and a light chain amino acid sequence comprising a CDRL1 of SEQ ID NO: 4, a CDRL2 of SEQ ID NO: 5, and a CDRL3 of SEQ ID NO: 6; wherein the composition comprises 1-90% main isoform.

55. A composition comprising a main isoform of an antibody, wherein the main isoform comprises a heavy chain amino acid sequence comprising a CDRH1 of SEQ ID NO: 1, a CDRH2 of SEQ ID NO: 2, and a CDRH3 of SEQ ID NO: 3, and a light chain amino acid sequence comprising a CDRL1 of SEQ ID NO: 4, a CDRL2 of SEQ ID NO: 5, and a CDRL3 of SEQ ID NO: 6; wherein the composition comprises ≥1%, main isoform.

56. A composition comprising a charged variant of the antibody comprising a heavy chain amino acid sequence comprising a CDRH1 of SEQ ID NO: 1, a CDRH2 of SEQ ID NO: 2, and a CDRH3 of SEQ ID NO: 3, and a light chain amino acid sequence comprising a CDRL1 of SEQ ID NO: 4, a CDRL2 of SEQ ID NO: 5, and a CDRL3 of SEQ ID NO: 6; wherein the composition comprises: ≤70%, acidic variant; and/or ≤30%, basic variant; and/or ≥1%, main isoform.

### EXAMPLES

### Example 1: Determination of Percent DL Species and Average DAR in an ADC Composition

For the Examples 2-9, percent DL and average DAR were calculated as follows:

The percent of a specific DL species (e.g. percent DL0, percent DL2, percent DL4a, percent DL4b, percent DL6, and percent DL8) was determined by separating individual DL species using hydrophobic interaction chromatography (HIC) (as exemplified in FIG. 2), calculating the area under the curve for each DL peak, and dividing each DL peak by the total area under the curve for all DL species combined.

The average DAR for each sample was calculated from the area under the curve of each DL species by using the following formula:

$$\% DL {Component}_{X} = \frac{{A}_{X}}{{A}_{0} + {A}_{1} + {A}_{2} + {A}_{3} + {A}_{4 a} + {A}_{4 b} + {A}_{5} + {A}_{6} + {A}_{8} + {A}_{10}} \times 100$$

$$DAR = \frac{\begin{matrix}( {A}_{1} \times 2 ) + ( {A}_{2} \times 2 ) + ( {A}_{3} \times 2 ) + ( {A}_{4 a} \times 4 ) + \\ ( {A}_{4 b} \times 4 ) + ( {A}_{5} \times 6 ) + ( {A}_{6} \times 6 ) + ( {A}_{8} \times 8 ) \times ( {A}_{10} \times 8 )\end{matrix}}{{A}_{0} + {A}_{1} + {A}_{2} + {A}_{3} + {A}_{4 a} + {A}_{4 b} + {A}_{5} + {A}_{6} + {A}_{8} + {A}_{10}}$$

$$Where : \\ {A}_{X} = Peak area of loading X peak \\ X = {A}_{0} , {A}_{1} , {A}_{2} , {A}_{3} , {A}_{4 a} , {A}_{4 b} , {A}_{5} , {A}_{6} , {A}_{8} , and {A}_{10}$$

$${A}_{0} , {A}_{1} , {A}_{2} , {A}_{3} , {A}_{4 a} , {A}_{4 b} , {A}_{5} , {A}_{6} , {A}_{8} , and {A}_{10} = is the peak of DL 0 , DL 1 , DL 2 , DL 3 , \\ DL 4 a , DL 4 b , DL 5 , DL 6 , DL 8 and DL 10 peaks \\ ( only including peaks \geq DL ( 0.08 \% ) )$$

Reference standards used in Examples 2-8, and which could be used for other experiments as well, include the samples in Table 1.

TABLE 1

| Lot | 132371424 | 162397940 | 182407660 | 108M4203 |
|---|---|---|---|---|
| Drug | % DL0: 2.9% | % DL0: 2.8% | % DL0: 4.7% | % DL0: 3.8% |
| Load | % DL1: 0.7% | % DL1: 0.7% | % DL1: 0.4% | % DL1: 0.4% |
| Variants | % DL2: 19.6% | % DL2: 19.1% | % DL2: 26.0% | % DL2: 23.2% |
| | % DL3: 1.7% | % DL3: 2.4% | % DL3: 1.7% | % DL3: 1.3% |
| | % DL4a: 38.0% | % DL4: 37.7% | % DL4a: 36.3% | % DL4a: 37.9% |
| | % DL4b: 9.4% | % DL4b: 8.7% | % DL4b: 7.5% | % DL4b: 7.5% |
| | % DL5: 3.0% | % DL5: 2.6% | % DL5: 2.9% | % DL5: 3.6% |
| | % DL6: 16.6% | % DL6: 17.9% | % DL6: 14.2% | % DL6: 15.2% |
| | % DL8: 7.9% | % DL8: 8.0% | % DL8: 6.2% | % DL8: 6.9% |
| DAR | 4.1 | 4.2 | 3.8 | 4.0 |

### Example 2: Impact of Average DAR on Cell Growth Inhibition

The cell growth inhibition of belantamab mafodotin was determined by measuring cell viability of NCI-H929 cells, a human multiple myeloma cell line, after 48 hour incubation with belantamab mafodotin. The cell viability was measured using Promega's CellTiter Glo technology. Increasing concentration of belantamab mafodotin proportionally corresponded to a decrease in CellTiter Glo luminescence signal. The dose response (EC50) was generated by SoftMax Pro using a 4-parameter nonlinear regression logistic model. The ratio of reference standard #132371424 EC50 to the sample EC50 was calculated to determine the relative potency. The results are summarized in Table 2.

TABLE 2

| Average | Relative |
|---|---|
| DAR Value | Potency |
| 2.1 | 0.5 |
| 3.0 | 0.7 |
| 3.5 | 0.8 |
| 4.1 | 1.0 |
| 4.6 | 1.1 |
| 5.0 | 1.1 |
| 5.7 | 1.3 |

### Example 3: Impact of Average DAR on ADCC Activity

Belantamab mafodotin, multiple myeloma cells, and NK-cells (effector cells) were incubated. Without being bound by theory, belantamab mafodotin bound to BCMA expressed on the multiple myeloma cell surface, and Fc region of the antibody bound to FcγRIIIa on the effector cells through their FcγRIIIa receptor. Engagement of these receptors on the surface of the effector cells resulted in the synthesis and secretion of cytokines (IFNg), and release of granules (perforin and granzymes) that enter into the cytoplasm of the target cells. The granzymes initiated a signalling event within the target cells that caused the death of these cells by apoptosis. The source of NK-cells was Peripheral Blood Mononuclear Cells (PBMC), which are isolated from human whole blood. Then, 1 mL of NCI-H929 cells, human multiple myeloma cell line, are loaded with 10 μL of an acetoxymethyl ester of a fluorescence enhancing ligand, BATDA (bis-(acetoxymethyl) 2,2′:6′,2″-terpyridine-6,6″-dicarboxylate) (Perkin-Elmer cat no. C136-100), which penetrated the cell membrane. The ester bonds in BATDA are hydrolyzed to form a hydrophilic ligand (TDA), which no longer can pass through the cell membrane. The labeled cells were added to varying amounts of belantamab mafodotin and effector cells (PBMCs). The cells lyse and release TDA. After cytolysis, this ligand was combined with 200 μL DELFIA Europium Solution (Perkin-Elmer cat no. C135-100) to form a highly fluorescent and stable chelate (EuTDA). The measured fluorescence correlated directly with the amount of lysed cells when measured with a fluorescence plate reader. The ADCC activity of belantamab mafodotin was reported as a ratio of sample EC50 value versus that of the reference standard #132371424. The results are summarized in Table 3.

TABLE 3

| Average | Relative |
|---|---|
| DAR Value | Potency |
| 2.1 | 1.1 |
| 3.0 | 0.8 |
| 3.5 | 1.0 |
| 4.6 | 0.8 |
| 5.0 | 1.1 |
| 5.7 | 0.8 |

### Example 4: Impact of Average DAR on BCMA Binding and FcγRIIIa Binding

The binding of BCMA and FcγRIIIa by belantamab mafodotin was measured using surface plasmon resonance (SPR). Belantamab mafodotin was diluted to 10 μg/mL with PBST, injected and captured by protein A immobilized on a CM5 sensor chip. BCMA was then injected and bound to the captured belantamab mafodotin. Next, FcγRIIIa was injected and bound to the captured belantamab mafodotin. The functional concentrations of belantamab mafodotin binding to BCMA and FcγRIIIa was calculated from a reference standard curve (reference standard #132371424) and reported as the BCMA or FcγRIIIa binding concentration, respectively. The total belantamab mafodotin concentration of the sample was pre-determined by absorbance at 280 nm. The specific binding activity (%) was calculated by dividing the BCMA or FcγRIIIa binding concentration by the absorbance at 280 nm concentration. The results are summarized in Table 4.

TABLE 4

| Average | BCMA | FcγRIIIa |
|---|---|---|
| DAR Value | Binding (%) | (%) |
| 2.1 | 101 | 106 |
| 3.0 | 95 | 98 |
| 3.5 | 100 | 103 |
| 4.6 | 89 | 89 |
| 5.0 | 94 | 94 |
| 5.7 | 94 | 92 |

### Example 5: Impact of Average DAR on Tumor Volume

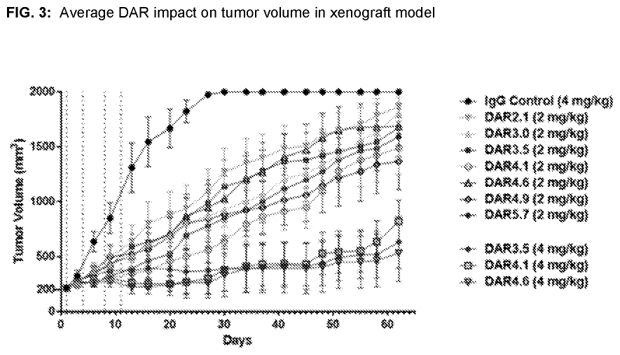

A multiple myeloma cell line was subcutaneously implanted in the flank of severe combined immunodeficiency (SCID) mice. From around day 15 onwards, all tumors were measured thrice weekly using a caliper system, and length and width of each mouse tumor were recorded to calculate tumor volume (volume=length×(width2)×0.5). When the mean tumor volume reached ˜200 mm3, mice were randomized into groups and dosed with belantamab mafodotin with one of the average DAR samples twice weekly for 2 weeks. All tumors were measured in this manner, and individual mice were euthanized once their tumor reached a mean tumor measurement of 2.0 mm3 or at day 60, whichever came first. The summary of the study design is summarized in Table 5 and the results are depicted in FIG. 3

TABLE 5

| Average | |
|---|---|
| DAR Value | Dose |
| 2.1 | 2 mg/kg |
| 3.0 | 2 mg/kg |
| 3.5 | 2 mg/kg |
| 4.1 | 2 mg/kg |
| 4.6 | 2 mg/kg |
| 4.9 | 2 mg/kg |
| 5.7 | 2 mg/kg |
| 3.5 | 4 mg/kg |
| 4.1 | 4 mg/kg |
| 4.6 | 4 mg/kg |

### Example 6: Impact of DL Species on BCMA Binding, FcγRIIIa Binding, and FcRn Binding

Samples of belantamab mafodotin comprising specific DL species were prepared by collected individual peaks of a HIC chromatogram. The binding of BCMA and FcγRIIIa of belantamab mafodotin was measured using surface plasmon resonance (SPR) as described in Example 4.

The binding of Neonatal Fc Receptor (FcRn) to belantamab mafodotin was measured using surface plasmon resonance (SPR). The samples were diluted and belantamab mafodotin was captured by FcRn, which was immobilized on a nitrilotriacetic acid (NTA) sensor chip. The FcRn binding concentration of the sample was determined by interpolation of the binding response on a calibration curve. Specific binding activity (%) was calculated by dividing the FcRn binding concentration by the total protein concentration. The results are summarized in Table 6.

Reference standard #162397940 was used.

TABLE 6

| DAR | BCMA | FcγRIIIa | FcRn |
|---|---|---|---|
| Species | (%) | (%) | (%) |
| Control | 96 | 97 | 101 |
| DL0 | 103 | 110 | 108 |
| DL2 | 99 | 104 | 109 |
| DL4a | 95 | 103 | 102 |
| DL4b | 90 | 91 | 99 |
| DL6 | 89 | 84 | 97 |
| DL8 | 86 | 82 | 93 |

### Example 7: Impact of DL Species on Cell Growth Inhibition

Specific DL specie samples of belantamab mafodotin were prepared as in Example 7. Cell growth inhibition was determined as in Example 2. Reference standard #162397940 was used. The results are summarized in Table 7.

TABLE 7

| DAR | Relative |
|---|---|
| Species | Potency |
| DL0 | 0.0 |
| DL2 | 0.5 |
| DL4a | 0.9 |
| DL4b | 1.0 |
| DL6 | 1.6 |
| DL8 | 1.8 |

### Example 8: Impact of DL Species on ADCC Activity

Belantamab mafodotin bound to BCMA expressed on a multiple myeloma cell surface. The Fc region of belantamab mafodotin bound to FcγRIIIa (CD16a) on Jurkat T effector cells (Promega, Cat #G7102, BioCat #140011) that have been engineered to stably express 1) the human FcγRIIIa receptors V158 high affinity variant and 2) a Luciferase Reporter Gene fused to the promoter downstream of NFAT activation sequences. When the antibody bound simultaneously to H929 and effector cells, activation of the NFAT pathway results in gene transcription from the luciferase reporter gene and expression of firefly luciferase within the effector cells. After addition of a luminescent substrate (Bio-Glo™ Luciferase Assay System, Promega, Cat #G7940) and cell lysis occurs, the luciferase produced due to NFAT activation is measured as a Relative Luminescent Units (RLUs) using a plate reader. In this assay, belantamab mafodotin was added in a dose dependent manner; therefore, the dose response (half maximal effective concentration or EC50) was generated using a nonlinear regression logistic model. The ratio of reference standard EC50 to the sample EC50 was calculated to determine the relative potency. Reference standard #162397940 was used. The results are summarized in Table 8.

TABLE 8

| | ADCC |
|---|---|
| DL | Relative |
| Species | Potency |
| DL0 | 1.1 |
| DL2 | 1.3 |
| DL4a | 1.3 |
| DL4b | 1.0 |
| DL6 | 0.9 |
| DL8 | 0.7 |

### Example 9: Average DAR and Percent DL Species for Several Lots of Belantamab Mafodotin

Several (19) batches of belantamab mafodotin were manufactured. Average DAR and percent DL species were calculated for each lot as described in Example 1. The results are summarized in Tables 9-12.

TABLE 9

| | Lot 1 | Lot 2 | Lot 3 | Lot 4 | Lot 5 |
|---|---|---|---|---|---|
| DL0 | 2.0 | 3.5 | 4.0 | 3.9 | 3.9 |
| DL2 | 15.8 | 21.2 | 24.5 | 24.7 | 24.6 |
| DL4a | 37.1 | 36.1 | 36.4 | 35.8 | 35.7 |
| DL4b | 7.1 | 8.2 | 7.4 | 8.3 | 8.5 |
| DL6 | 19.1 | 16.3 | 15.1 | 15.2 | 15.2 |
| DL8 | 12.0 | 8.1 | 7.1 | 7.1 | 7.0 |
| Ave DAR | 4.5 | 4.1 | 3.9 | 3.9 | 3.9 |

TABLE 10

| | Lot 6 | Lot 7 | Lot 8 | Lot 9 | Lot 10 |
|---|---|---|---|---|---|
| DL0 | 4.0 | 4.3 | 3.8 | 3.7 | 3.9 |
| DL2 | 24.7 | 25.4 | 24.4 | 24.1 | 23.9 |
| DL4a | 35.7 | 36.5 | 36.9 | 37.9 | 36.4 |
| DL4b | 8.4 | 7.6 | 7.7 | 7.1 | 7.6 |
| DL6 | 15.1 | 14.2 | 15.5 | 15.6 | 15.1 |
| DL8 | 7.0 | 6.1 | 6.9 | 6.9 | 7.5 |
| Ave DAR | 3.9 | 3.9 | 4.0 | 4.0 | 4.0 |

TABLE 11

| | Lot 11 | Lot 12 | Lot 13 | Lot 14 | Lot 15 |
|---|---|---|---|---|---|
| DL0 | 4.8 | 4.0 | 4.2 | 4.2 | 4.5 |
| DL2 | 26.3 | 24.1 | 24.8 | 24.8 | 25.8 |
| DL4a | 35.9 | 36.2 | 36.2 | 35.9 | 36.6 |
| DL4b | 7.6 | 7.8 | 7.7 | 8.0 | 7.5 |
| DL6 | 14.0 | 15.0 | 14.6 | 14.6 | 14.3 |
| DL8 | 6.1 | 7.3 | 6.8 | 6.9 | 6.0 |
| Ave DAR | 3.8 | 4.0 | 3.9 | 3.9 | 3.8 |

TABLE 12

| | Lot 16 | Lot 17 | Lot 18 | Lot 19 |
|---|---|---|---|---|
| DL0 | 4.1 | 4.1 | 4.2 | 4.0 |
| DL2 | 24.6 | 24.5 | 24.7 | 24.2 |
| DL4a | 36.3 | 35.5 | 35.6 | 35.8 |
| DL4b | 7.5 | 8.1 | 8.0 | 8.1 |
| DL6 | 14.7 | 14.5 | 14.3 | 15.0 |
| DL8 | 7.1 | 7.2 | 7.1 | 7.3 |
| Ave DAR | 3.9 | 3.9 | 3.9 | 4.0 |

### Example 10: Belantamab Mafodotin Forced Degradation Study Design

A summary of the study design for Examples 11-18 is depicted in Table 13.

TABLE 13
Belantamab mafodotin Forced Degradation Study Design

| | Predominant | | |
|---|---|---|---|
| | Quality | | |
| Stressor | Attribute | Condition | Time Points |
| 1. Oxidative | Oxidation | 500:1 molar ratio | 1, 3, 16, and 24 hours |
| Conditions | | (H₂O₂:ADC) | |
| | | incubation at | |
| | | 25° C./50% RH¹ | |
| 2. Chemical: | Deamidation | pH 9.0 at 25° C./ | 3, 7, 14, and 21 days |
| High pH (base | | 50% RH | |
| treated) | | | |
| 3. Chemical: | Isomerization | pH 5.0 at 25° C./ | 7, 14, and 21 days |
| Low pH (acid | | 50% RH | |
| treated) | | | |
| 4. Thermal: | Isomerization | 40° C./75% RH | 7, 14, 21, and 28 |
| Elevated | | | days |
| Temperature | | | |
| 5. Photo | Oxidation | Caron | 300 kLux-hours, 50 |
| Exposure | | Photostability | watts/m²; (0.25 × ICH) |
| | | Chamber | 600 kLux-hours, 100 |
| | | Temperature: | watts/m²; (0.5 × ICH) |
| | | 25 ± 5° C. | 1200 kLux-hours, 200 |
| | | | watts/m²; (1 × ICH) |
| | | | 1800 kLux-hours, 300 |
| | | | watts/m²; (1.5 × ICH) |

¹RH: relative humidity

### Example 11: Oxidative Conditions

To create oxidative stress samples, belantamab mafodotin samples were diluted to 10 mg/mL and hydrogen peroxide was added so that the molar ratio of hydrogen peroxide to belantamab mafodotin was 500:1. Samples were quenched with methionine and buffer exchanged using a 3 kDa molecular weight cut off filter (MWCO).

Deamidation and oxidation was determined using tryptic peptide mapping tandem mass spectrometry (peptide mapping LC-MS/MS). The sample was denatured in 6M guanidine HCl to a concentration of 4.2 μg/μL. The disulfide bonds were reduced with 50 mM DTT for 20 minutes at room temperature. Iodoacetate was added at 100 mM and reacted with the free cysteine residues for 30 minutes at room temperature, protected from light. The sample was buffer exchanged using BioRad spin columns (part no. 7326221) before digestion with Worthington trypsin (part no. TRTPCK) at 0.5% trypsin for 15 minutes at 37° C. The resulting peptides were loaded onto a Waters reversed phase ultra performance liquid chromatography (UPLC) column (part no. 186003687) and eluted with a water and acetonitrile gradient in 0.1% trifluoroacetic acid using a Waters Acquity UPLC. The peptides were detected with UV detector and a mass spectrometer, such as Thermo Scientific LTQ Orbitrap XL. The extracted ion chromatograms of the unmodified and modified peptides were used to calculate the levels of either deamidation or oxidation by dividing the area under the curve of the modified peptide by the total areas under the curve for both modified and unmodified peptides.

The binding of BCMA and FcγRIIIa by belantamab mafodotin was measured using surface plasmon resonance (SPR) as described in Example 4. The binding of Neonatal Fc Receptor (FcRn) to belantamab mafodotin was measured using surface plasmon resonance (SPR) as described in Example 6.

Reference standard #182407660A was used. A summary of the results is depicted in Table 14 and Table 15.

TABLE 14
PTM abundance in H₂O₂-treated belantamab mafodotin

| | | Hours | |
|---|---|---|---|
| Post-Translational Modification | Lot | 0 | 24 |
| HC N388 | SLBZ4036 | 0.8 | 3.5 |
| % Deamidation | SLBW3623 | 0.9 | 3.5 |
| | SLBZ7108 | 0.7 | 3.0 |
| HC M34 (CDRH1) | SLBZ4036 | 0.2 | 45.1 |
| % Oxidation | SLBW3623 | 0.4 | 45.4 |
| | SLBZ7108 | 0.2 | 45.5 |
| HC M256 | SLBZ4036 | 2.9 | 98.6 |
| % Oxidation | SLBW3623 | 2.9 | 98.7 |
| | SLBZ7108 | 3.5 | 98.3 |
| HC M432 | SLBZ4036 | 0.4 | 94.8 |
| % Oxidation | SLBW3623 | 0.6 | 95.2 |
| | SLBZ7108 | 0.6 | 94.9 |

TABLE 15
Binding activity of H₂O₂-treated belantamab mafodotin

| | | Hours | | | | |
|---|---|---|---|---|---|---|
| Property | Lot | 0 | 1 | 3 | 16 | 24 |
| % Antigen Specific Binding | SLBZ4036 | 92 | 99 | 94 | 67 | 62 |
| | SLBW3623 | 88 | 99 | 97 | 74 | 68 |
| | SLBZ7108 | 99 | 99 | 98 | 68 | 64 |
| % FcγRIIIa Specific Binding | SLBZ4036 | 91 | 93 | 87 | 70 | 66 |
| | SLBW3623 | 89 | 96 | 92 | 74 | 70 |
| | SLBZ7108 | 99 | 95 | 92 | 70 | 67 |
| % FcRn Specific Binding | SLBZ4036 | 93 | 85 | 73 | 55 | 52 |
| | SLBW3623 | 97 | 86 | 79 | 58 | 57 |
| | SLBZ7108 | 95 | 85 | 76 | 57 | 56 |

Extrapolating from the data for oxidation at HC Met 34, up to 37% oxidation can result in at least 70% antigen specific binding activity. This is calculated using a linear slope for Time 0 (0.2-0.4%) and 24 hours (45.1-45.5%) M34 oxidation samples, which have 88-99% and 62-68% antigen specific binding activity, respectively.

Extrapolating from the data for oxidation at HC Met 256, up to 89% oxidation can result in at least 70% FcγRIIIa specific binding activity. This is calculated using a linear slope for Time 0 (2.9-3.5%) and 24 hours (98.3-98.7%) M256 oxidation samples, which have 89-99% and 66-70% FcγRIIIa specific binding activity, respectively. Extrapolating from the data for oxidation at HC Met 256, up to 64% oxidation can result in at least 70% FcRn specific binding activity. This is calculated using a linear slope for Time 0 (2.9-3.5%) and 24 hours (98.3-98.7%) M256 oxidation samples, which have 93-97% and 52-57% FcRn specific binding activity, respectively.

Extrapolating from the data for oxidation at HC Met 432, up to 86% oxidation can result in at least 70% FcγRIIIa specific binding activity. This is calculated using a linear slope for Time 0 (0.4-0.6%) and 24 hours (94.8-95.2%) M432 oxidation samples, which have 88-99% and 66-70% FcγRIIIa specific binding activity, respectively. Extrapolating from the data for oxidation at HC Met 432, up to 61% oxidation can result in at least 70% FcRn specific binding activity. This is calculated using a linear slope for Time 0 (0.4-0.6%) and 24 hours (94.8-95.2%) M432 oxidation samples, which have 93-97% and 52-57% FcRn specific binding activity, respectively.

### Example 12: Chemical: High pH (Base Treated)

To create high pH samples, belantamab mafodotin samples were diluted with a 4 mM Tris buffer to adjust the pH to 9. Samples were further diluted to 10 mg/mL and incubated at 25° C./50% RH for up to 21 days.

Deamidation, isomerization, and oxidation was determined using tryptic peptide mapping tandem mass spectrometry (peptide mapping LC-MS/MS) as described in Example 11. The extracted ion chromatograms of the unmodified and modified peptides were used to calculate the levels of either deamidation, isomerization, or oxidation by dividing the area under the curve of the modified peptide by the total areas under the curve for both modified and unmodified peptides

The binding of BCMA and FcγRIIIa by belantamab mafodotin was measured using surface plasmon resonance (SPR) as described in Example 4. The binding of Neonatal Fc Receptor (FcRn) to belantamab mafodotin was measured using surface plasmon resonance (SPR) as in Example 6.

Reference standard #182407660A was used. The results are summarized in Table 16 and Table 17.

TABLE 16
PTM abundance in base-treated belantamab mafodotin

| Post-Translational | | Days | |
|---|---|---|---|
| Modification | Lot | 0 | 21 |
| HC N31 | SLBZ4036 | 0.1 | 0.9 |
| % Deamidation | SLBW3623 | 0.1 | 1.0 |
| | SLBZ7108 | 0.0 | 1.0 |
| HC N388 | SLBZ4036 | 0.8 | 10.3 |
| % Deamidation | SLBW3623 | 0.9 | 10.2 |
| | SLBZ7108 | 0.7 | 10.0 |
| HC N393 | SLBZ4036 | 0.9 | 13.9 |
| % Deamidation | SLBW3623 | 1.2 | 14.5 |
| | SLBZ7108 | 0.9 | 14.5 |
| HC D103 | SLBZ4036 | 4.4 | 5.5 |
| % Aspartic Acid | SLBW3623 | 4.5 | 5.6 |
| Isomerization | SLBZ7108 | 4.1 | 5.4 |
| HC M256 | SLBZ4036 | 2.9 | 4.6 |
| % Oxidation | SLBW3623 | 2.9 | 5.1 |
| | SLBZ7108 | 3.5 | 5.3 |
| HC M432 | SLBZ4036 | 0.4 | 1.2 |
| % Oxidation | SLBW3623 | 0.6 | 2.0 |
| | SLBZ7108 | 0.6 | 2.1 |

TABLE 17
Binding activity of base-treated belantamab mafodotin

| | | Days | | | | |
|---|---|---|---|---|---|---|
| Property | Lot | 0 | 3 | 7 | 14 | 21 |
| % Antigen Specific Binding | SLBZ4036 | 92 | 94 | 91 | 89 | 87 |
| | SLBW3623 | 88 | 93 | 90 | 87 | 85 |
| | SLBZ7108 | 99 | 96 | 94 | 92 | 89 |
| % FcγRIIIa Specific Binding | SLBZ4036 | 91 | 91 | 90 | 88 | 87 |
| | SLBW3623 | 89 | 93 | 89 | 87 | 86 |
| | SLBZ7108 | 99 | 94 | 92 | 90 | 89 |
| % FcRn Specific Binding | SLBZ4036 | 93 | — | — | — | 91 |
| | SLBW3623 | 97 | — | — | — | 90 |
| | SLBZ7108 | 95 | — | — | — | 91 |

It is expected that deamidation at HC Asn 388 and HC Asn 393 can go higher than the reported levels of 10.3% and 14.5% respectively, without any impact to Antigen Specific Binding, FcγRIIIa Specific Binding and FcRn Specific Binding.

### Example 13: Chemical: Low pH (Acid Treated)

To create low pH samples, belantamab mafodotin samples were diluted with a citrate buffer to adjust the pH to 5. Samples were further diluted to 10 mg/mL and incubated at 25° C./50% RH for up to 21 days.

Isomerization, deamidation and oxidation was determined using tryptic peptide mapping tandem mass spectrometry (peptide mapping LC-MS/MS) as described in Example 11. The extracted ion chromatograms of the unmodified and modified peptides were used to calculate the levels of either isomerization, deamidation or oxidation by dividing the area under the curve of the modified peptide by the total areas under the curve for both modified and unmodified peptides

The binding of BCMA and FcγRIIIa by belantamab mafodotin was measured using surface plasmon resonance (SPR) as described in Example 4. The binding of Neonatal Fc Receptor (FcRn) to belantamab mafodotin was measured using surface plasmon resonance (SPR) as in Example 6.

Reference standard #182407660A was used. The results are summarized in Table 18 and Table 19.

TABLE 18
PTM abundance in acid-treated belantamab mafodotin

| Post-Translational | | Days | |
|---|---|---|---|
| Modification | Lot | 0 | 21 |
| HC N388 | SLBZ4036 | 0.8 | 2.0 |
| % Deamidation | SLBW3623 | 0.9 | 2.1 |
| | SLBZ7108 | 0.7 | 1.7 |
| HC N393 | SLBZ4036 | 0.9 | 1.7 |
| % Deamidation | SLBW3623 | 1.2 | 1.9 |
| | SLBZ7108 | 0.9 | 1.6 |
| HC D103 | SLBZ4036 | 0.3 | 7.8 |
| % Succinimide | SLBW3623 | 0.4 | 7.5 |
| | SLBZ7108 | 0.3 | 7.5 |
| HC M256 | SLBZ4036 | 2.9 | 9.7 |
| % Oxidation | SLBW3623 | 2.9 | 9.3 |
| | SLBZ7108 | 3.5 | 9.2 |
| HC M432 | SLBZ4036 | 0.4 | 2.2 |
| % Oxidation | SLBW3623 | 0.6 | 2.4 |
| | SLBZ7108 | 0.6 | 2.2 |

TABLE 19
Binding activity of acid-treated belantamab mafodotin

| | | Days | | | |
|---|---|---|---|---|---|
| Property | Lot | 0 | 7 | 14 | 21 |
| % Antigen Specific Binding | SLBZ4036 | 92 | 86 | 86 | 81 |
| | SLBW3623 | 88 | 86 | 84 | 79 |
| | SLBZ7108 | 99 | 91 | 86 | 83 |
| % FcγRIIIa Specific Binding | SLBZ4036 | 91 | 93 | 95 | 94 |
| | SLBW3623 | 89 | 90 | 93 | 91 |
| | SLBZ7108 | 99 | 96 | 95 | 96 |
| % FcRn Specific Binding | SLBZ4036 | 93 | — | — | 95 |
| | SLBW3623 | 97 | — | — | 94 |
| | SLBZ7108 | 95 | — | — | 97 |

### Example 14. Thermal: Elevated Temperature

To create thermal stress samples, belantamab mafodotin samples were diluted in the formulation buffer to 10 mg/mL and incubated at 40° C./75% RH for up to 28 days.

Isomerization, deamidation, and oxidation was determined using tryptic peptide mapping tandem mass spectrometry (peptide mapping LC-MS/MS) as described in Example 11. The extracted ion chromatograms of the unmodified and modified peptides were used to calculate the levels of either isomerization, deamidation, or oxidation by dividing the area under the curve of the modified peptide by the total areas under the curve for both modified and unmodified peptides

The binding of BCMA and FcγRIIIa by belantamab mafodotin was measured using surface plasmon resonance (SPR) as described in Example 4. The binding of Neonatal Fc Receptor (FcRn) to belantamab mafodotin was measured using surface plasmon resonance (SPR) as in Example 6.

Reference standard #182407660A was used. The results are summarized in Table 20 and Table 21.

TABLE 20
PTM abundance in thermal-treated belantamab mafodotin

| Post-Translational | | Days | |
|---|---|---|---|
| Modification | Lot | 0 | 28 |
| HC N329 | SLBZ4036 | 0.1 | 5.7 |
| % Deamidation | SLBW3623 | 0.0 | 5.7 |
| | SLBZ7108 | 0.1 | 5.2 |
| HC N388 | SLBZ4036 | 0.8 | 3.7 |
| % Deamidation | SLBW3623 | 0.9 | 3.9 |
| | SLBZ7108 | 0.7 | 3.6 |
| HC N393 | SLBZ4036 | 0.9 | 3.5 |
| % Deamidation | SLBW3623 | 1.2 | 3.4 |
| | SLBZ7108 | 0.9 | 3.2 |
| HC D103 | SLBZ4036 | 0.3 | 3.0 |
| % Succinimide | SLBW3623 | 0.4 | 2.9 |
| | SLBZ7108 | 0.3 | 3.0 |
| HC D103 | SLBZ4036 | 4.4 | 29.0 |
| % Aspartic Acid Isomerization | SLBW3623 | 4.5 | 29.3 |
| | SLBZ7108 | 4.1 | 28.7 |
| HC M256 | SLBZ4036 | 2.9 | 5.5 |
| % Oxidation | SLBW3623 | 2.9 | 5.6 |
| | SLBZ7108 | 3.5 | 6.0 |

TABLE 21
Binding activity of thermal-treated belantamab mafodotin

| | | Days | | | | |
|---|---|---|---|---|---|---|
| Property | Lot | 0 | 7 | 14 | 21 | 28 |
| % Antigen Specific | SLBZ4036 | 92 | 78 | 71 | 61 | 55 |
| Binding | SLBW3623 | 88 | 86 | 77 | 67 | 62 |
| | SLBZ7108 | 99 | 85 | 76 | 67 | 61 |
| % FcγRIIIa Specific | SLBZ4036 | 91 | 87 | 87 | 83 | 83 |
| Binding | SLBW3623 | 89 | 97 | 96 | 93 | 94 |
| | SLBZ7108 | 99 | 94 | 93 | 91 | 91 |
| % FcRn Specific Binding | SLBZ4036 | 93 | — | — | — | 89 |
| | SLBW3623 | 97 | — | — | — | 91 |
| | SLBZ7108 | 95 | — | — | — | 93 |

Extrapolating from the data for isomerization at HC Asp 130, up to 23% isomerization can result in at least 70% antigen specific binding activity. This is calculated using a linear slope for Time 0 (4.1-4.4%) and 28 days (28.7-29.3%) D103 isomerization samples, which have 88-99% and 55-62% antigen specific binding activity, respectively.

### Example 15 Photo Exposure

To create photo exposed sample, belantamab mafodotin samples were diluted to 10 mg/mL and filled into glass vials and transferred to a Caron Photostability Chamber at 25° C. for varying degrees of photo exposure shown in Table 10.

Oxidation, deamidation, and isomerization was determined using tryptic peptide mapping tandem mass spectrometry (peptide mapping LC-MS/MS) as described in Example 11. The extracted ion chromatograms of the unmodified and modified peptides were used to calculate the levels of either Oxidation, deamidation, or isomerization by dividing the area under the curve of the modified peptide by the total areas under the curve for both modified and unmodified peptides

The binding of BCMA and FcγRIIIa by belantamab mafodotin was measured using surface plasmon resonance (SPR) as described in Example 4. The binding of Neonatal Fc Receptor (FcRn) to belantamab mafodotin was measured using surface plasmon resonance (SPR) as in Example 6.

Reference standard #182407660A was used. The results are summarized in Table 22 and Table 23.

TABLE 22
PTM abundance in photo-treated belantamab mafodotin

| Post-Translational | | | 1.5 × |
|---|---|---|---|
| Modification | Lot | Control | ICH |
| HC N388 | SLBZ4036 | 0.8 | 3.4 |
| % Deamidation | SLBW3623 | 0.9 | 3.6 |
| | SLBZ7108 | 0.7 | 3.0 |
| HC N393 | SLBZ4036 | 0.9 | 2.4 |
| % Deamidation | SLBW3623 | 1.2 | 2.4 |
| | SLBZ7108 | 0.9 | 2.0 |
| HC D103 | SLBZ4036 | 0.3 | 1.3 |
| % Succinimide | SLBW3623 | 0.4 | 1.3 |
| | SLBZ7108 | 0.3 | 1.2 |
| HC M34 (CDRH1) | SLBZ4036 | 0.2 | 3.1 |
| % Oxidation | SLBW3623 | 0.4 | 3.5 |
| | SLBZ7108 | 0.2 | 3.7 |
| HC M256 | SLBZ4036 | 2.9 | 27.7 |
| % Oxidation | SLBW3623 | 2.9 | 29.1 |
| | SLBZ7108 | 3.5 | 30.9 |
| HC M432 | SLBZ4036 | 0.4 | 18.4 |
| % Oxidation | SLBW3623 | 0.6 | 20.6 |
| | SLBZ7108 | 0.6 | 22.7 |

TABLE 23
Binding activity of photo-treated belantamab mafodotin

| | | | 0.25X | 0.5X | 1X | 1.5X |
|---|---|---|---|---|---|---|
| Property | Lot | Control | ICH | ICH | ICH | ICH |
| % Antigen Specific | SLBZ4036 | 92 | 89 | 80 | 69 | 60 |
| Binding | SLBW3623 | 88 | 88 | 81 | 68 | 63 |
| | SLBZ7108 | 99 | 90 | 83 | 72 | 62 |
| % FcγRIIIa Specific | SLBZ4036 | 91 | 93 | 85 | 79 | 73 |
| Binding | SLBW3623 | 89 | 91 | 87 | 81 | 77 |
| | SLBZ7108 | 99 | 93 | 90 | 84 | 78 |
| % FcRn Specific | SLBZ4036 | 93 | — | — | — | 78 |
| Binding | SLBW3623 | 97 | — | — | — | 81 |
| | SLBZ7108 | 95 | — | — | — | 77 |

### Example 16 C-Terminal Cleavage and N-Terminal Pyroglutamic Acid

Several lots of belantamab were analyzed for levels of N-terminal pyroglutamic acid and C-terminal cleavage using tryptic peptide mapping tandem mass spectrometry (peptide mapping LC-MS/MS) as described in Example 11. The extracted ion chromatograms of the unmodified and modified peptides were used to calculate the levels of either C-terminal cleavage and N-terminal pyroglutamic acid by dividing the area under the curve of the modified peptide by the total areas under the curve for both modified and unmodified peptides

The binding of BCMA and FcγRIIIa by belantamab was measured using surface plasmon resonance (SPR) as described in Example 4. The binding of Neonatal Fc Receptor (FcRn) to belantamab was measured using surface plasmon resonance (SPR) as in Example 6.

Reference standards used were #122368059 and #172405900. The results are summarized in Table 24-25.

TABLE 24
Pyro-glutamate and lysine cleaved abundance in
belantamab and corresponding activity

| Lot | 172405773 | 182407670 | 182408599 | 182408314 | 182408902 | 182409958 |
|---|---|---|---|---|---|---|
| HC Q1- | 100.0 | 100.0 | 100.0 | 100.0 | 100.0 | 100.0 |
| Pyroglutamic | | | | | | |
| acid | | | | | | |
| HC K451- | 88.1 | 90.2 | 90.3 | 88.5 | 89.2 | 89.3 |
| Lysine | | | | | | |
| Cleavage | | | | | | |
| Antigen | 100 | 104 | 104 | 106 | 106 | 105 |
| Specific | | | | | | |
| binding | | | | | | |
| activity (%): | | | | | | |
| FcγRIIIa | 101 | 104 | 103 | 105 | 105 | 104 |
| Specific | | | | | | |
| binding | | | | | | |
| activity (%) | | | | | | |

TABLE 25
Pyro-glutamate and lysine cleaved abundance in
belantamab and corresponding activity

| Lot | 122368059 | 122370432 | 152390946 | 162399241 | 172405900 |
|---|---|---|---|---|---|
| HC Q1 - | 99.9 | 99.9 | 99.9 | 99.9 | 99.9 |
| Pyroglutamic acid | | | | | |
| HC K451 - | 93.0 | 96.7 | 94.9 | 95.9 | 90.4 |
| Lysine Cleavage | | | | | |
| Antigen Specific | 92 | 91 | 91 | 90 | 96 |
| binding activity | | | | | |
| (%): | | | | | |
| FcγRIIIa Specific | 93 | 93 | 94 | 94 | 100 |
| binding activity (%) | | | | | |

### Example 17: Glycosylation

Several lots of belantamab were analyzed for glycosylation patterns. The profiles were determined using Ultra Performance Liquid Chromatography (UPLC) with Hydrophilic Interaction Liquid Chromatography (HILIC) separation and fluorescence detection. The samples were diluted to a concentration of 10 μg/μL with water, and the glycans were released from belantamab by an enzymatic digestion with PNGaseF using a PNGase F kit from New England BioLabs, (Cat #P0705L). The glycans released by PNGase F were labelled with anthranilamide (Sigma-Aldrich, Cat #A89804). The labelled glycans were then purified to remove excess labelling solution using a HILIC column step; the glycans were loaded and washed with water and eluting with acetonitrile. The labelled glycans were then separated using a Waters Glycan BEH Amide column (cat no. 186004742) on a Waters Acquity UPLC with an ammonium formate/formic acid and acetonitrile gradient. The glycans were detected using fluorescence detection with excitation at 365 nm and emission at 438 nm. Quantitation of the glycans was achieved by dividing the area under the curve of a glycan by the total area under the curve for all detected glycans.

The reference standard used was #122368059. The results are summarized in Table 26-27.

TABLE 26
Glycosylation patterns of belantamab

| Lot | 172405773 | 182407670 | 182408599 | 182408314 | 182408902 | 182409958 |
|---|---|---|---|---|---|---|
| % G0 | 62.3 | 63.6 | 62.7 | 55.5 | 57.8 | 56.5 |
| % G1 | 26.0 | 25.2 | 25.5 | 30.8 | 29.3 | 29.8 |
| G0-GlcNAc | 1.1 | 1.0 | 1.0 | 0.9 | 0.9 | 1.0 |

TABLE 27
Glycosylation patterns of belantamab

| Lot | 122368059 | 122370432 | 152390946 | 162399241 | 172405900 |
|---|---|---|---|---|---|
| % G0 | 69.3 | 77.5 | 76.0 | 78.0 | 60.2 |
| % G1 | 13.7 | 8.9 | 11.7 | 11.2 | 26.2 |
| G0- | 4.1 | 5.3 | 3.2 | 3.2 | 1.1 |
| GlcNAc | | | | | |

### Example 18: Glycoengineering

The impact of glyco-enriched samples of belantamab on ADCC activity and binding was determined. ADCC activity was measured as in Example 3. BCMA and FcγRIIIa binding was determined as in Example 4.

For the galactosylation experiments, glycosylation was measured with reduced LC-MS Sample was diluted to 1 mg/mL, and 50 uL of 1M DTT was added and reacted at either 25C or 37 C for 30 minutes before analysis on a mass spectrometer that could include a Micromass Q-tof. Heavy and light chains were separated using size exclusion chromatography with isocratic flow of water, acetonitrile, and trifluoroacetic acid. The spectra for each heavy and light chain were summed and deconvoluted using MaxEnt software from Waters. The major glycoforms were detected and relative amounts were estimated from signal counts or area under the curve. The results are summarized in Table 28-29.

TABLE 28
Glyco-enriched samples of belantamab and corresponding activity

| | Biological | Specific | Specific |
|---|---|---|---|
| | Activity by | BCMA | FcγRIIIa |
| | ADCC | binding | binding |
| | Reporter | activity | activity |
| Control | 0.9 | 94 | 96 |
| G0 enriched | 1.0 | 98 | 100 |
| β-N-Acetylglucosaminidase | 1.6 | 96 | 97 |
| G0-GlcNAc enriched | | | |
| Deglycosylated with PNGaseF | 0.1 | 95 | 8 |
| Control | 1.1 | 100 | 103 |
| G0 enriched | 0.9 | 97 | 98 |
| G0-GlcNAc enriched | 0.9 | 100 | 99 |
| G0-2GlcNAc enriched | 0.8 | 101 | 97 |
| Control | 1.0 | 98 | 102 |
| Galactosylation G1 enriched | 1.0 | 97 | 97 |
| Galactosylation G1, G2 enriched | 1.1 | 95 | 94 |
| Galactosylation 1 hr | | 98 | 99 |
| Galactosylation 2 hr | | 94 | 95 |
| Galactosylation 4 hr | | 93 | 94 |

TABLE 29
Abundance (%) of glyco-enriched samples of belantamab

| | | | | G0- | G0- |
|---|---|---|---|---|---|
| | G0 | G1 | G2 | GlcNAc | 2GlcNAc |
| Control | 79.7 | 11.5 | ND | ND | ND |
| G0 enriched | 92.0 | 0.9 | ND | ND | ND |
| β-N-Acetylglucosaminidase | 42.0 | 6.9 | ND | 29.1 | ND |
| G0-GlcNAc enriched | | | | | |
| Deglycosylated with PNGaseF | n/a | n/a | n/a | n/a | n/a |
| Control | 76.0 | 23.2 | ND | 0 | ND |
| G0 enriched | 99.2 | 0 | ND | 0.8 | ND |
| G0-GlcNAc enriched | 8.9 | 3.2 | ND | 43 | 38.5 |
| G0-2GlcNAc enriched | 0 | 0 | ND | 3.2 | 88.1 |
| Control | 63 | 26.1 | 3 | ND | ND |
| Galactosylation G1 enriched | 16 | 58 | 24 | ND | ND |
| Galactosylation G1,G2 | 5 | 54 | 39 | ND | ND |
| enriched | | | | | |
| Galactosylation 1 hr | 0 | 31 | 67 | ND | ND |
| Galactosylation 2 hr | 0 | 14 | 83 | ND | ND |
| Galactosylation 4 hr | 0 | 5 | 92 | ND | ND |

### Example 19: Tolerable Ranges

Tolerable ranges (70-130% activity) were determined by using data from Tables 11 through Table 18 for the first and last timepoints (either Day 21 or Day 28, as relevant for the condition). Binding data were plotted against the relative percentages of the relevant post-translational modification to determine the slope of the relationship. Using this information, the predicted level of each post-translational modification was calculated for a binding measurement of at least 70%. Results of this extrapolation are summarized in Table 30. Trends reported broadly reflect observations seen with either belantamab or belantamab mafodotin.

TABLE 30
Extrapolation of data for functional variants of belantamab
and belantamab mafodotin

| | Product-related | | | | | |
|---|---|---|---|---|---|---|
| | substances | | | | | |
| | | Tolerable | | | | |
| | | range | Product-related impurities | | | |
| | | resulting | | Tolerable | Tolerable | Tolerable |
| | | in 70- | | range | range | range |
| | Reference | 130% | Reference | resulting | resulting | resulting |
| | standard % | activity | standard % | in 70- | in 70- | in 70- |
| | (forced | (antigen/ | (forced | 130% | 130% | 130% |
| Post | degradation | FcγRIIIa/ | degradation | activity | activity | activity |
| translational | maximal % | FcRn | maximal % | (antigen | (FcγRIIIa | (FcRn |
| modification | tested) | binding) | tested) | binding) | binding) | binding) |
| Isomerization | | | HC D103 ≤ | ≤23% | | |
| | | | 7% (29.3%) | | | |
| Oxidation | | | HC M34 ≤ | ≤37% | | |
| | | | 2% | | | |
| | | | (45.5%) | | | |
| | | | HC M256 ≤ | | ≤89% | ≤64% |
| | | | 5% | | | |
| | | | (98.7%) | | | |
| | | | HC M432 ≤ | | ≤86% | ≤61% |
| | | | 2% | | | |
| | | | (95.2%) | | | |
| Deamidation | HC N388 ≤ | 0-100% | | | | |
| | 2% | | | | | |
| | (10.3%) | | | | | |
| | HC N393 ≤ | 0-100% | | | | |
| | 2% | | | | | |
| | (14.5%) | | | | | |
| Clipping | HC C- | 0-100% | | | | |
| | terminal | | | | | |
| | lysine 88.1- | | | | | |
| | 96.7% | | | | | |
| | cleaved | | | | | |
| Cyclization | HC N- | 0-100% | | | | |
| | terminal pyro- | | | | | |
| | glutamate | | | | | |
| | 100% | | | | | |
| Glycosylation | G0 55.5- | 0-100% | | | | |
| | 80.0% | | | | | |
| | (99.2%) | | | | | |
| | G1 8.9-30.8% | 0-100% | | | | |
| | (58%) | | | | | |
| | G2 Difficult to | 0-100% | | | | |
| | detect/resolve | | | | | |
| | (39-92%) | | | | | |
| | G0-GlcNac | 0-100% | | | | |
| | 0.9-5.5% | | | | | |
| | (43%) | | | | | |
| | G0-2GlcNac | 0-100% | | | | |
| | Difficult to | | | | | |
| | detect/resolve | | | | | |
| | (38.5-88.1%) | | | | | |

### Example 20: Degradation Product Summary

The prevalence of degradation products observed under different forced degradation conditions which have no impact on belantamab mafodotin activity are summarized below (Table 31).

TABLE 31
Degradation Products which do not Impact the Potency of belantamab mafodotin

| | Level of Degradants Observed Having no Impact on antigen binding | | | |
|---|---|---|---|---|
| | | | | Antigen specific |
| Condition | Isomerization | Deamidation | Oxidation | binding |
| Unstressed | HC Asp-iso | HC Asn 388 = | HC Met 34 = | 88-99% |
| | 103 | 0.7-0.9% | 0.2-0.4% | |
| | 4.1-4.4% | HC Asn 393 = | HC Met 256 = | |
| | HC Asp-suc | 0.9-1.2% | 2.9-3.5% | |
| | 103 | | HC Met 432 = | |
| | 0.3-0.4% | | 0.4-0.6% | |
| Oxidative | | HC Asn 388 = | HC Met 34 = | 88-99% (T0) |
| (Peroxide | | 0.7-0.9% | 0.2-0.4% | |
| oxidation) | | HC Asn 393 = | HC Met 256 = | |
| | | 0.9-1.2% | 2.9-3.5% | |
| | | | HC Met 432 = | |
| | | | 0.4-0.6% | |
| Chemical: | HC Asp-iso | HC Asn 388 = | HC Met 256 = | 85-89% (pH 9.0, |
| high pH | 103 | 10.0-10.3% | 4.6-5.3% | 21 days) |
| (Base | 5.4-5.6% | HC Asn 393 = | HC Met 432 = | |
| treated) | | 13.9-14.5% | 1.2-2.1% | |
| | | HC Asn 31 = 1% | | |
| Chemical: | HC Asp-suc | HC Asn 388 = | HC Met 256 = | 79-83% (pH 5.0, |
| low pH | 103 | 1.7-2.1% | 9.2-9.7% | 21 days) |
| (Acid | 7.5-7.8% | HC Asn 393 < | HC Met 432 = | |
| treated) | | 1.6-1.9% | 2.2-2.4% | |
| Thermal: | HC Asp-iso | HC Asn 388 = | HC Met 256 = | 88-99% (T0) |
| elevated | 103 | 0.7-0.9% | 2.9-3.5% | |
| temperature | 4.1-4.4% | HC Asn 393 = | | |
| | HC Asp-suc | 0.9-1.2% | | |
| | 103 | HC Asn 329 = | | |
| | 0.3-0.4% | 0.1% | | |
| Photo | HC Asp-suc | HC Asn 388 = | HC Met 34 = | 88-99% (T0) |
| exposure | 103 | 0.7-0.9% | 0.2-0.4% | |
| | 0.3-0.4% | HC Asn 393 = | HC Met 256 = | |
| | | 0.9-1.2% | 32.9-.5% | |
| | | | HC Met 432 = | |
| | | | 0.4-0.6% | |

### Example 21

A summary of the study design for belantamab forced degradation in Examples 21 to 26 is depicted in Table 32. Methods were broadly similar to those described in Examples 11-18 above for belantamab mafodotin (except where stated).

TABLE 32
Belantamab Forced Degradation Study Design

| | Predominant | | |
|---|---|---|---|
| | Quality | | |
| Stressor | Attribute | Condition | Time Points |
| 1. Oxidative | Oxidation | 500:1 molar ratio | 1, 3, 16, and 24 hours |
| Conditions | | (H₂O₂:mAb) | |
| | | incubation at | |
| | | 25° C./60% RH¹ | |
| 2. Chemical: | Deamidation | pH 9.0 at 25° C./ | 3, 7, 14, 21 and 28 |
| High pH | | 50% RH | days |
| (base | | | |
| treated) | | | |
| 3. Chemical: | Fragmentation | pH 3.5 at 25° C./ | 3, 7, 14, 21 and 28 |
| Low pH | and | 50% RH | days |
| (acid | isomerization | | |
| treated) | | | |
| 4. Thermal: | Fragmentation, | 40° C./75% RH | 3, 7, 14, 21 and 28 |
| Elevated | aggregation and | | days |
| Temperature | isomerization | | |
| 5. Photo | Aggregation | Suntest | 300 kLux-hours, 50 |
| Exposure | and | XLS Light | watts/m²; (0.25 × ICH) |
| | oxidation | Chamber with | 600 kLux-hours, 100 |
| | | xenon light. | watts/m²; (0.5 × ICH) |
| | | Temperature: | 1200 kLux-hours, 200 |
| | | 25 ± 5° C. | watts/m²; (1 × ICH) |
| | | | 1800 kLux-hours, 300 |
| | | | watts/m²; (1.5 × ICH) |

¹RH: relative humidity

### Example 22: Belantamab Oxidative Conditions

Oxidation of HC M256 increased from approximately 2% to approximately 98%, and M432 increased from approximately 1% to approximately 96% after 24 hours. There was a 21-25% decrease in specific binding for FcγRIIIa and a 10-18% decrease in FcRn binding by SPR. Oxidation in the Fc likely alters the binding activity of belantamab for FcγRIIIa and FcRn. Oxidation of HC M34 in the CDR1 increased from approximately 0.3% to 47.7-48.5% after 24 hours, resulting in no change to antigen binding, within assay variability. Cysteine and tryptophan oxidation levels were low throughout this study, and no other significant post-translational modifications were detected.

### Example 23: Belantamab Base-Treated

An increase from approximately 3.5% to approximately 6.5% in HC D103 isomerization was observed after 28 days. An increase from 0.1% to approximately 2.5% in HC N31; an increase from approximately 2.0% to approximately 10% in HC N388, and an increase from approximately 1.7% to approximately 18.5% in HC N393 deamidation after 28 days. In addition, an increase from approximately 2.2% to approximately 3.7% in HC M256 oxidation was also observed in pH 9-stressed 28 day belantamab. cIEF analysis showed an increase from approximately 25% to approximately 62% acidic variant; and a decrease from approximately 9% to approximately 4.5% basic variant (see Table 33 below). All changes observed were within assay variability for antigen, FcγRIIIa, and FcRn specific binding; therefore, binding by SPR is comparable for pH 9.0-stressed belantamab at day 28.

TABLE 33
cIEF results for base-treated belantamab

| | | Days | | | | | |
|---|---|---|---|---|---|---|---|
| Variant | Lot | 0 | 3 | 7 | 14 | 21 | 28 |
| % total | 172402762 | 24.8 | 30.1 | 37.5 | 46.9 | 55.4 | 61.4 |
| acidic | 182411532 | 25.2 | 31.1 | 37.1 | 45.8 | 56.2 | 63.9 |
| | 182411322 | 25.9 | 26.6 | 37.7 | 45.9 | 55.2 | 63.4 |
| % main | 172402762 | 67.4 | 62.5 | 56.1 | 48.3 | 39.9 | 33.9 |
| | 182411532 | 65.1 | 60.9 | 55.1 | 46.7 | 38.7 | 31.2 |
| | 182411322 | 64.7 | 62.9 | 54.7 | 47.1 | 39.4 | 32.5 |
| % total | 172402762 | 7.9 | 7.4 | 6.4 | 4.8 | 4.7 | 4.7 |
| basic | 182411532 | 9.6 | 8.0 | 7.8 | 7.5 | 5.0 | 4.9 |
| | 182411322 | 9.4 | 10.5 | 7.5 | 6.9 | 5.3 | 4.1 |

### Example 24: Belantamab Acid-Treated

Fragment increased from 0.8% to 2.3-2.7% after 28 days. An increase from 0.2% to 4.0% in succinimide formation at HC D103, and an increase from approximately 3.5% to approximately 5.8% in HC D103 isomerization was observed after 28 days. In addition to aspartic acid isomerization, an increase from approximately 2.2% to approximately 3.7% in HC M256 oxidation was also observed in pH 3.5-stressed 28 day belantamab. cIEF analysis showed an increase from approximately 25% to approximately 28% acidic variant; and an increase from approximately 9% to approximately 13% basic variant (see Table 34 below). All changes observed in antigen, FcγRIIIa, and FcRn specific binding were within assay variability; therefore, binding by SPR is comparable for pH 3.5-stressed belantamab after 28 days.

TABLE 34
cIEF results for acid treated belantamab

| | | Days | | | | | |
|---|---|---|---|---|---|---|---|
| Variant | Lot | 0 | 3 | 7 | 14 | 21 | 28 |
| % total | 172402762 | 24.8 | 26.3 | 26.8 | 27.5 | 27.5 | 27.5 |
| acidic | 182411532 | 25.2 | 26.3 | 27.4 | 27.7 | 28.8 | 30.0 |
| | 182411322 | 25.9 | 31.2 | 25.7 | 26.6 | 27.5 | 27.9 |
| % main | 172402762 | 67.4 | 64.8 | 63.4 | 61.5 | 61.5 | 58.5 |
| | 182411532 | 65.1 | 63.8 | 60.5 | 58.8 | 59.3 | 57.5 |
| | 182411322 | 64.7 | 60.4 | 62.2 | 60.8 | 59.8 | 59.2 |
| % total | 172402762 | 7.9 | 8.9 | 9.8 | 11.0 | 11.1 | 14.0 |
| basic | 182411532 | 9.6 | 9.9 | 12.2 | 13.5 | 11.9 | 12.5 |
| | 182411322 | 9.4 | 8.4 | 12.1 | 12.6 | 12.7 | 13.0 |

### Example 25: Belantamab Thermal Treated

Fragment increased from 0.8% to 2.2-2.3% after 28 days. % aggregate did not change. An increase from 0.2% to 2.2% in succinimide formation at HC D103, and an increase from approximately 3.5% to approximately 29% in HC D103 isomerization was observed after 28 days. In addition to aspartic acid isomerization, an increase from approximately 2.2% to approximately 5.3% in HC M256, and an increase from approximately 1% to 2% in HC M432 oxidation were also observed in thermal-stressed 28 day belantamab. An increase from approximately 0% to approximately 7% in HC N329, an increase from approximately 2.0% to approximately 2.5% in HC N388, and increase from approximately 1.7% to approximately 2.6% in HC N393 deamidation after 28 days. Antigen specific binding was reduced to 63-67% at 28 days, consistent with the increase in HC D103 isomerization, which has been shown to impact antigen binding. Changes observed in FcγRIIIa and FcRn specific binding were within assay variability.

### Example 26: Belantamab Photo-Treated

Fragment increased from 0.8% to 1.5% at 1.5×ICH, and aggregate increased from approximately 1% to 6.5-7.5%. An increase from approximately 0.3% to 1.6-2.1% in HC M34, an increase from approximately 2.2% to 18.4-25.1% in HC M256, and increase from approximately 1% to 13.7-19.5% in HC M432 oxidation at 1.5×ICH. cIEF analysis showed an increase from approximately 25% to approximately 34% acidic variant; and no change in basic variant (see Table 35 below). All changes observed in antigen, FcγRIIIa, and FcRn specific binding were within assay variability; therefore, binding by SPR is comparable for pH 3.5-stressed belantamab after 28 days.

TABLE 35
cIEF results for photo treated belantamab

| | | Treatment | | | | |
|---|---|---|---|---|---|---|
| | | | 0.25X | 0.5X | 1X | 1.5X |
| Variant | Lot | Control | ICH | ICH | ICH | ICH |
| % total | 172402762 | 24.8 | 24.9 | 25.0 | 30.9 | 33.9 |
| acidic | 182411532 | 25.2 | 28.2 | 30.0 | 31.2 | 34.9 |
| | 182411322 | 25.9 | 27.8 | 29.2 | 32.2 | 34.4 |
| % main | 172402762 | 67.4 | 66.9 | 66.6 | 60.8 | 58.9 |
| | 182411532 | 65.1 | 61.8 | 60.9 | 59.2 | 56.6 |
| | 182411322 | 64.7 | 62.2 | 61.1 | 57.8 | 56.3 |
| % total | 172402762 | 7.9 | 8.2 | 8.4 | 8.3 | 7.2 |
| basic | 182411532 | 9.6 | 10.0 | 9.1 | 9.6 | 8.6 |
| | 182411322 | 9.4 | 10.0 | 9.7 | 10.0 | 9.3 |

The belantamab forced degradation study shows consistent results with the belantamab mafodotin forced degradation study presented above, with the only exception that up to 48.5% oxidation at HC M34 resulted in no change to antigen binding, within assay variability.

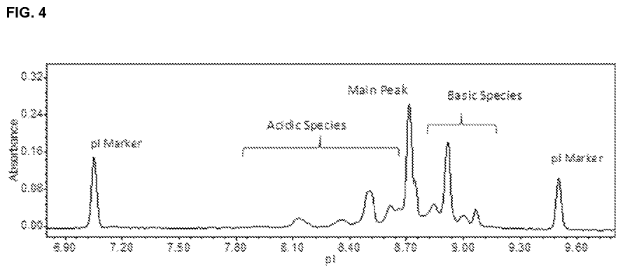

It is noted that no cIEF data is presented for belantamab mafodotin because the drug load contributes to the charge profile, whereas for belantamab, cIEF effectively separates the acid and basic variants from the main species (see FIG. 4).

SEQUENCE LISTING
SEQ. ID. NO. 1 - CDRH1
NYWMH
SEQ. ID. NO. 2: CDRH2
ATYRGHSDTYYNQKFKG
SEQ. ID. NO. 3: CDRH3
GAIYDGYDVLDN
SEQ. ID. NO. 4: CDRL1
SASQDISNYLN
SEQ. ID. NO. 5: CDRL2
YTSNLHS
SEQ. ID. NO. 6: CDRL3
QQYRKLPWT
SEQ. ID. NO. 7: heavy chain variable region
(CDRs are underlined)
QVQLVQSGAEVKKPGSSVKVSCKASGGTFSNYWMHWVRQAPGQGLEWMGA
TYRGHSDTYYNQKFKGRVTITADKSTSTAYMELSSLRSEDTAVYYCARGA
IYDGYDVLDNWGQGTLVTVSS
SED. ID. NO. 8: light chain variable region
(CDRs are underlined)
DIQMTQSPSSLSASVGDRVTITCSASQDISNYLNWYQQKPGKAPKLLIYY
TSNLHSGVPSRFSGSGSGTDFTLTISSLQPEDFATYYCQQYRKLPWTFGQ
GTKLEIKR
SEQ. ID. NO. 9: heavy chain region
(CDRs are underlined)
QVQLVQSGAEVKKPGSSVKVSCKASGGTFSNYWMHWVRQAPGQGLEWMGA
TYRGHSDTYYNQKFKGRVTITADKSTSTAYMELSSLRSEDTAVYYCARGA
IYDGYDVLDNWGQGTLVTVSSASTKGPSVFPLAPSSKSTSGGTAALGCLV
KDYFPEPVTVSWNSGALTSGVHTFPAVLQSSGLYSLSSVVTVPSSSLGTQ
TYICNVNHKPSNTKVDKKVEPKSCDKTHTCPPCPAPELLGGPSVFLFPPK
PKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREP
QVYTLPPSRDELTKNQVSLTCLVKGFYPSDIAVEWESNGQPENNYKTTPP
VLDSDGSFFLYSKLTVDKSRWQQGNVFSCSVMHEALHNHYTQKSLSLSPG
K
SEQ. ID. NO. 10: light chain region
(CDRs are underlined)
DIQMTQSPSSLSASVGDRVTITCSASQDISNYLNWYQQKPGKAPKLLIYY
TSNLHSGVPSRFSGSGSGTDFTLTISSLQPEDFATYYCQQYRKLPWTFGQ
GTKLEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC
SEQ. ID. NO. 11: heavy chain region with D103N
(CDRs are underlined)
QVQLVQSGAEVKKPGSSVKVSCKASGGTFSNYWMHWVRQAPGQGLEWMGA
TYRGHSDTYYNQKFKGRVTITADKSTSTAYMELSSLRSEDTAVYYCARGA
IYNGYDVLDNWGQGTLVTVSSASTKGPSVFPLAPSSKSTSGGTAALGCLV
KDYFPEPVTVSWNSGALTSGVHTFPAVLQSSGLYSLSSVVTVPSSSLGTQ
TYICNVNHKPSNTKVDKKVEPKSCDKTHTCPPCPAPELLGGPSVFLFPPK
PKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREP
QVYTLPPSRDELTKNQVSLTCLVKGFYPSDIAVEWESNGQPENNYKTTPP
VLDSDGSFFLYSKLTVDKSRWQQGNVFSCSVMHEALHNHYTQKSLSLSPG
K
SEQ. ID. NO. 12: heavy chain region with N388D
(CDRs are underlined)
QVQLVQSGAEVKKPGSSVKVSCKASGGTFSNYWMHWVRQAPGQGLEWMGA
TYRGHSDTYYNQKFKGRVTITADKSTSTAYMELSSLRSEDTAVYYCARGA
IYDGYDVLDNWGQGTLVTVSSASTKGPSVFPLAPSSKSTSGGTAALGCLV
KDYFPEPVTVSWNSGALTSGVHTFPAVLQSSGLYSLSSVVTVPSSSLGTQ
TYICNVNHKPSNTKVDKKVEPKSCDKTHTCPPCPAPELLGGPSVFLFPPK
PKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREP
QVYTLPPSRDELTKNQVSLTCLVKGFYPSDIAVEWESDGQPENNYKTTPP
VLDSDGSFFLYSKLTVDKSRWQQGNVFSCSVMHEALHNHYTQKSLSLSPG
K
SEQ. ID. NO. 13: heavy chain region with N393D
(CDRs are underlined)
QVQLVQSGAEVKKPGSSVKVSCKASGGTFSNYWMHWVRQAPGQGLEWMGA
TYRGHSDTYYNQKFKGRVTITADKSTSTAYMELSSLRSEDTAVYYCARGA
IYDGYDVLDNWGQGTLVTVSSASTKGPSVFPLAPSSKSTSGGTAALGCLV
KDYFPEPVTVSWNSGALTSGVHTFPAVLQSSGLYSLSSVVTVPSSSLGTQ
TYICNVNHKPSNTKVDKKVEPKSCDKTHTCPPCPAPELLGGPSVFLFPPK
PKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREP
QVYTLPPSRDELTKNQVSLTCLVKGFYPSDIAVEWESNGQPEDNYKTTPP
VLDSDGSFFLYSKLTVDKSRWQQGNVFSCSVMHEALHNHYTQKSLSLSPG
K
SEQ. ID. NO. 14: heavy chain region with N388D and
N393D (CDRs are underlined)
VQLVQSGAEVKKPGSSVKVSCKASGGTFSNYWMHWVRQAPGQGLEWMGAT
YRGHSDTYYNQKFKGRVTITADKSTSTAYMELSSLRSEDTAVYYCARGAI
YDGYDVLDNWGQGTLVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKVEPKSCDKTHTCPPCPAPELLGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQ
VYTLPPSRDELTKNQVSLTCLVKGFYPSDIAVEWESDGQPEDNYKTTPPV
LDSDGSFFLYSKLTVDKSRWQQGNVFSCSVMHEALHNHYTQKSLSLSPGK

What is claimed is:

1.-56. (canceled)

57. A composition comprising anti-BCMA antibodies, wherein the anti-BCMA antibodies comprise a belantamab variant, wherein the belantamab variant comprises one or more of:
a. deamidation of a residue selected from the group consisting of N388 and N393 of SEQ ID NO:9;
b. oxidation at a residue selected from the group consisting of M34, M256, and M432 of SEQ ID NO:9;
c. an amino acid change of aspartic acid (D) to asparagine (N) at residue 103 of SEQ ID NO:9;
d. C-terminal lysine cleavage; or
e. N-terminal conversion of glutamine to pyroglutamic acid.

58. The composition of claim 57, wherein 0.1-40% of antibodies in the composition comprise the oxidation.

59. The composition of claim 57, wherein 0.1-25% of the antibodies comprise the amino acid change.

60. The composition of claim 58, wherein the oxidation is at residue M34 of SEQ ID NO:9.

61. The composition of claim 59, wherein the anti-BCMA antibodies comprise an anti-BCMA antibody comprising (i) a heavy chain amino acid sequence that is at least about 90% identical to the heavy chain amino acid sequence of SEQ ID NO:9 and (ii) the light chain amino acid sequence of SEQ ID NO:10.

62. The composition of claim 60, wherein the anti-BCMA antibodies comprise an anti-BCMA antibody comprising (i) a heavy chain amino acid sequence that is at least about 90% identical to the heavy chain amino acid sequence of SEQ ID NO:9 and (ii) the light chain amino acid sequence of SEQ ID NO:10.

63. The composition according to claim 57, wherein 0.1-100% of the belantamab variant in the composition comprises oxidation at residue M256.

64. The composition according to claim 57, wherein 0.1-100% of the belantamab variant in the composition comprises oxidation at residue M432.

65. The composition according to claim 57, wherein 0.1-100% of the belantamab variant in the composition comprises at least one selected from the group consisting of:
a. deamidation of a residue selected from the group consisting of N388 and N393 of SEQ ID NO:9;
b. oxidation at a residue selected from the group consisting of M34, M256, and M432 of SEQ ID NO:9;
c. an amino acid change of aspartic acid (D) to asparagine (N) at residue 103 of SEQ ID NO:9;
d. C-terminal lysine cleavage; or
e. N-terminal conversion of glutamine to pyroglutamic acid.

66. The composition according to claim 57, wherein the composition comprises any percentage of glycoforms G0, G1, G2, G0-GlcNac or G0-2GlcNac.

67. The composition according to claim 57, which comprises belantamab.

68. The composition according to claim 57, which comprises belantamab mafodotin.

69. The composition according to claim 57, wherein an anti-BCMA antibody is conjugated to a cytotoxic agent to form an antibody-drug-conjugate.

70. The composition of claim 69, wherein percent DL2 is at least about 30%, about 15% to about 27%, or about 15% to about 32%; percent DL4a is at least about 30%, about 35% to about 38%, or about 30% to about 40%; percent DL4b is at least about 5%, about 7% to about 9%, or about 5% to about 10%; percent DL6 is at least about 10%, about 14% to about 20%, or about 10% to about 20%; and/or DL8 is at least about 1%, about 6.0% to about 12.0%, or about 4% to about 15%.

71. The composition of claim 69, wherein the average DAR is about 3.4 to about 4.6.

72. The composition of claim 69, wherein percent DL0 is less than or equal to about 10% or about 5%.

73. A pharmaceutical composition comprising the composition of claim 57 and at least one pharmaceutically acceptable excipient.

74. A formulation comprising the pharmaceutical composition of claim 73 comprising an anti-BCMA antigen binding protein at about 20 mg/mL to about 60 mg/mL, citrate buffer at about 10 mM to about 30 mM, trehalose at about 120 mM to about 240 mM, EDTA at about 0.01 mM to about 0.1 mM, polysorbate 20 or polysorbate 80 at about 0.01% to about 0.05%, at a pH of about 5.9 to about 6.5.

75. The formulation of claim 74, comprising about 20 mg/mL, about 25 mg/mL, about 50 mg/mL, or about 60 mg/mL belantamab mafodotin, 25 mM citrate buffer, 200 mM trehalose, 0.05 mM disodium EDTA, 0.02% polysorbate 20 or polysorbate 80, at a pH of about 5.9 to about 6.5.

76. A method of treating cancer in a subject in need thereof, the method comprising:
administering to the subject a therapeutically effective amount of the composition of claim 57.
